(19) United States
(12) Patent Application Publication
(10) Pub. No.: US 2021/0230649 A1
(43) Pub. Date: Jul. 29, 2021

(54) Production of Carbon-Based Compounds from Cellulosic Feedstock Fermentation

(71) Applicant: Metropolitan Water Reclamation District of Greater Chicago, Chicago, IL (US)

(72) Inventor: Kamlesh Patel, Chicago, IL (US)

(21) Appl. No.: 17/155,830

(22) Filed: Jan. 22, 2021

Related U.S. Application Data
(60) Provisional application No. 62/965,592, filed on Jan. 24, 2020.

(51) Int. Cl. C12P 7/64; C12P 19/14

(52) U.S. Cl. CPC C12P 7/64; C12P 2201/00; C12P 19/14

(57) ABSTRACT

Provided herein are methods of producing carbon-based compounds from the fermentation of cellulosic feedstocks. In certain embodiments, the cellulosic feedstock and/or source of the cellulosic feedstock is grass, guar gum, leaves, cattails, and/or phragmites.

### CROSS-REFERENCE TO RELATED APPLICATIONS

This is a U.S. Non-Provisional Application that claims priority to U.S. Provisional Application No. 62/965,592, filed Jan. 24, 2020, which is incorporated herein by reference in its entirety.

### REFERENCE TO SEQUENCE LISTING SUBMITTED ELECTRONICALLY

The content of the electronically submitted sequence listing in ASCII text file (Name MWRD_205858_ST25.txt; Size: 742 bytes; and Date of Creation: Apr. 19, 2021) filed with the application is incorporated herein by reference in its entirety.

### INCORPORATION OF TABLES

The Tables in APPENDICES A through H, filed herewith in the files:
“APPENDIX_A_grass_aerobic_bacteria.pdf”;
“APPENDIX_B_grass_aerobic_all_species.pdf”;
“APPENDIX_C_grass_anaerobic_bacteria.pdf”;
“APPENDIX_D_grass_anaerobic_all_species.pdf”;
“APPENDIX_E_guar_gum_aerobic_bacteria.pdf”;
“APPENDIX_F_guar_gum_aerobic_all_species.pdf”;
“APPENDIX_G_guar_gum_anaerobic_bacteria.pdf”; and
“APPENDIX_H_guar_gum_anaerobic_all_species.pdf”
   are part of the disclosure of this application and are incorporated herein in their entireties.

### BACKGROUND

Currently used wastewater treatment processes require the utilization of carbon sources. Many available carbon sources, however, are problematic because of insufficient availability, high cost, logistics of transport, need for infrastructure modification, potential for odors, energy input needed, non-sustainable nature, environmentally unfriendly nature, uncertainty over quality, uncertainty over consistency, uncertainty over long-term availability, and/or other undesirable characteristics.

Thus, there remains a need to develop methods of producing carbon-based compounds in the form of volatile fatty acids (VFAs)—such as but not limited to for use in wastewater treatment—in a manner that addresses current shortcomings in available carbon sources.

### SUMMARY

Provide for herein is a method of producing a volatile fatty acid that comprises fermenting a cellulosic feedstock present in a fermentation solution, wherein the fermentation solution comprises cellulolytic microorganisms and the cellulosic feedstock. In certain embodiments, the cellulosic feedstock comprises grass, leaves, phragmites, cattails, guar gum, or a combination thereof. In certain embodiments, the cellulosic feedstock comprises grass or guar gum. In certain embodiments, the cellulolytic microorganisms are supplied to the fermentation solution in an inoculum comprising the cellulolytic microorganisms. In certain embodiments, the composition of identities of and/or the metabolic pathways utilized by the cellulolytic microorganisms supplied to the fermentation solution have been previous adapted to the type of cellulosic feedstock and/or fermentation conditions to be used. In certain embodiments, the cellulosic feedstock is fermented under aerobic conditions. In certain embodiments, the cellulosic feedstock is fermented under anaerobic conditions.

In certain embodiments, the cellulosic feedstock comprises grass and the fermentation is aerobic; optionally wherein the grass is not switchgrass. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 26A. In certain embodiments, the cellulolytic bacterial microorganisms in the fermentation solution and/or in the inoculum have been adapted to fermentation of grass under aerobic conditions.

In certain embodiments, the cellulosic feedstock comprises grass and the fermentation is aerobic; optionally wherein the grass is not switchgrass. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 27A. In certain embodiments, the cellulolytic microorganisms in the fermentation solution and/or in the inoculum have been adapted to fermentation of grass under aerobic conditions.

In certain embodiments, the cellulosic feedstock comprises grass and the fermentation is anaerobic; optionally wherein the grass is not switchgrass. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 28A. In certain embodiments, the cellulolytic bacterial microorganisms in the fermentation solution and/or in the inoculum have been adapted to fermentation of grass under anaerobic conditions.

In certain embodiments, the cellulosic feedstock comprises grass and the fermentation is anaerobic; optionally wherein the grass is not switchgrass. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 29A. In certain embodiments, the cellulolytic microorganisms in the fermentation solution and/or in the inoculum have been adapted to fermentation of grass under anaerobic conditions.

In certain embodiments, the cellulosic feedstock comprises guar gum and the fermentation is aerobic. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic microorganisms in Table 30A. In certain embodiments, the cellulolytic bacterial microorganisms in the fermentation solution and/or in the inoculum have been adapted to fermentation of guar gum under aerobic conditions.

In certain embodiments, the cellulosic feedstock comprises guar gum and the fermentation is aerobic. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 31A. In certain embodiments, the cellulolytic microorganisms in the fermentation solution and/or in the inoculum have been adapted to fermentation of guar gum under aerobic conditions.

In certain embodiments, the cellulosic feedstock comprises guar gum and the fermentation is anaerobic. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic microorganisms in Table 32A. In certain embodiments, the cellulolytic bacterial microorganisms in the fermentation solution and/or in the inoculum have been adapted to fermentation of guar gum under anaerobic conditions.

In certain embodiments, the cellulosic feedstock comprises guar gum and the fermentation is anaerobic. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 33A. In certain embodiments, the cellulolytic microorganisms in the fermentation solution and/or in the inoculum have been adapted to fermentation of guar gum under anaerobic conditions.

In certain of any of the above embodiments, the cellulolytic microorganisms in the inoculum are derived from a seed source reservoir. And, in certain embodiments, the seed source reservoir is an anaerobic mesophilic digester draw from a municipal sewage treatment operation. Further, certain embodiments provide for deriving cellulolytic microorganisms from a seed source reservoir for use in the fermentation of the cellulosic feedstock. In certain embodiments, the composition of the identities of and/or the metabolic pathways utilized by the cellulolytic microorganisms is adapted to the type of cellulosic feedstock and/or fermentation conditions to be used. Certain embodiments further comprise cultivating and maintaining the derived cellulolytic microorganisms composition for use as the inoculum.

Further provided for in this disclosure is a carbon dependent nutrient removal and/or recovery process comprising the use of a volatile fatty acid produced as described elsewhere herein.

Further provided for is an industrial process comprising the use of a volatile fatty acid produced as described elsewhere herein as its raw or intermediate material

### BRIEF DESCRIPTION OF THE DRAWINGS

FIG. 1 shows representative chemical composition of tall fescue grass straw.

FIG. 2 shows theoretical production of volatile fatty acids and biogas from cellulose fermentation.

FIG. 3 shows statistics generated in the determination of cellulolytic bacterial species.

FIG. 4 shows statistics generated in the determination of cellulolytic bacterial species.

FIG. 5 shows statistics generated in the determination of cellulolytic bacterial species.

FIG. 6 shows dominant taxa identified under two grass and two guar gum fermentation conditions.

FIG. 7 shows numerous cellulolytic bacterial taxa identified under two grass and two guar gum fermentation conditions, too numerous to list by name.

FIG. 8 shows alpha diversity among the cellulolytic bacterial species. Alpha diversity calculations were based on non-rarefied data. The calculations show that all samples are highly diverse at the “species” level with roughly 500 taxa observed per sample. The samples are pretty even (J′), so the Shannon index (H′) is pretty high for all of them.

FIG. 9 shows VFA concentrations measured using HACH's TNTplus 872 reagents (method 10240) (x-axis) and the GC-FID method, this method is used by the Calumet Analytical Laboratory (y-axis). These concentrations were plotted, along with linear and power trend lines, as well as the equation and R² value for each.

FIG. 10 shows predicted values for VFA concentrations plotted against the actual VFA concentration values returned by the Calumet Analytical Laboratory.

FIG. 11 shows predicted values for VFA concentrations plotted against the actual VFA concentration values returned by the Calumet Analytical Laboratory.

FIG. 12 shows predicted values for VFA concentrations plotted against the actual VFA concentration values returned by the Calumet Analytical Laboratory.

FIG. 13 shows predicted values for VFA concentrations plotted against the actual VFA concentration values returned by the Calumet Analytical Laboratory.

FIG. 14 shows predicted values for VFA concentrations plotted against the actual VFA concentration values returned by the Calumet Analytical Laboratory.

FIG. 15 shows values for VFA production from grass and guar gum (aka, plant extract (PE), as referred to) fermentation under aerobic conditions over time.

FIG. 16 shows values for VFA production from grass and guar gum fermentation under anaerobic conditions over time.

FIG. 17 shows values for biogas production of grass and guar gum fermentation under anaerobic conditions over time.

FIG. 18 shows the composition of biogas produced by anaerobic grass fermentation.

FIG. 19 shows the temperature effect on grass fermentation.

FIG. 20 shows the mixing effect on grass fermentation.

FIG. 21 show a change in VFA composition over fermentation time.

FIG. 22 lists the common and botanical name of a number of representative grasses useful in practicing this disclosure.

FIG. 23 shows VFA production at a loading rate of 4 g/L grass.

FIG. 24 shows VFA production at a loading rate of 6 g/L grass.

FIG. 25 shows results of average VFA yields.

FIG. 26 shows daily pH values in stock cultures. The information is presented below as Rank Sum Test box plot that graphs the percentiles and the median of column data. The ends of the boxes define the 25th and 75th percentiles, with a line at the median and error bars defining the 10th and 90th percentiles. Dashed line in the box is mean and solid line is median.

FIG. 27 shows a pH shift during upscale fermentation.

FIG. 28A and FIG. 28B show system characteristics of stock reactors; FIG. 28A represents system characteristics during derivation of cellulolytic organisms and preparation of stock culture while FIG. 28B represents system characteristics during maintenance of stock cultures and second phase serum bottle and upscale testing.

FIG. 29 shows temperature values in stock cultures. The information is presented below as Rank Sum Test box plot that graphs the percentiles and the median of column data. The ends of the boxes define the 25th and 75th percentiles, with a line at the median and error bars defining the 10th and 90th percentiles. Dashed line in the box is mean and solids line is median.

FIG. 30 shows energy usage for mixing the fermenter content in scaled-up fermenter experiments. Wherever the use of plant effluent is answered no; nutrient solution was added.

FIG. 31 shows VFA production at a loading rate of 2 g/L grass.

FIG. 32 shows a comparison of HACH vs ALD generated VFA data.

FIG. 33 shows a validation of HACH generated data.

### DETAILED DESCRIPTION

### Definitions

The terms defined immediately below are more fully defined by reference to the specification in its entirety. To the extent necessary to provide descriptive support, the subject matter and/or text of the appended claims is incorporated herein by reference in their entirety.

It will be understood by all readers of this disclosure that the exemplary aspects and embodiments described and claimed herein can be suitably practiced in the absence of any recited feature, element or step that is, or is not, specifically disclosed herein.

It is to be noted that the term “a” or “an” entity refers to one or more of that entity; for example, “a volatile fatty acid,” is understood to represent one or more volatile fatty acids. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, i.e., 1, 2, 3, or 4, the disclosure specifically includes any range in between the values, i.e., 1 to 3, 1 to 4, 2 to 4, etc.

The headings provided herein are solely for ease of reference and are not limitations of the various aspects or aspects of the disclosure, which can be had by reference to the specification as a whole.

As used herein, a “cellulosic feedstock” is a material comprising the polysaccharide cellulose (C₆H₁₀O₅), (where n can be 100s to 1000s) with β(1-4) linked D-glucose units and/or guar gum (guaran) C₁₀H₁₄N₅Na₂O₁₂P₃.

### OVERVIEW

Provided herein are methods of producing carbon-based compounds from the fermentation of cellulosic feedstocks. In certain embodiments, the cellulosic feedstock and/or source of the cellulosic feedstock is grass, guar gum, leaves, cattails, and/or phragmites. Advantages of such methods include environmentally sustainable carbon generation, sequestration of carbon from air and affixing in soil, filtering of water runoff and flood control, cooler urban environments, contributes to oxygen production, and providing a great environment for recreational activities. Additional advantages include better landscape and curb appeal resulting in enhanced aesthetic and property values, potential of no wastewater plant operations and compliance issues, reduction/elimination of odors, control of costs. In addition, in the context of municipal waste water management, there is the opportunity for increased public-private partnership, better public image and perception due to environment-friendly technology, chemical cost savings for carbon need, sustainable outlet for recycling nutrients via composting/P-recovery, a free substitute to wood chips in compost processing, production of biogas as a by-product furthering efforts to achieve energy neutrality, and reduced emission of green-house gases by avoiding carbon chemical manufacturing, transportation, etc.

Cellulolytic organisms (e.g., bacteria, archaea, and fungi) exist in anaerobically digested sludge in a very low density, feeding on cellulose-type sources (e.g., toilet paper and other cellulosic products) for their cellular carbon needs. In certain embodiments, these organisms are derived, cultivated, concentrated, and redirected to derive their cellular-carbon need from cellulosic feedstocks such as grass, guar gum, leaves, phragmites, and cattails. These cellulolytic organisms can quickly adapt and degrade, for example, grass and guar gum (both of which contain cellulose or cellulose-like complex carbohydrates in abundant quantities), for their carbon needs.

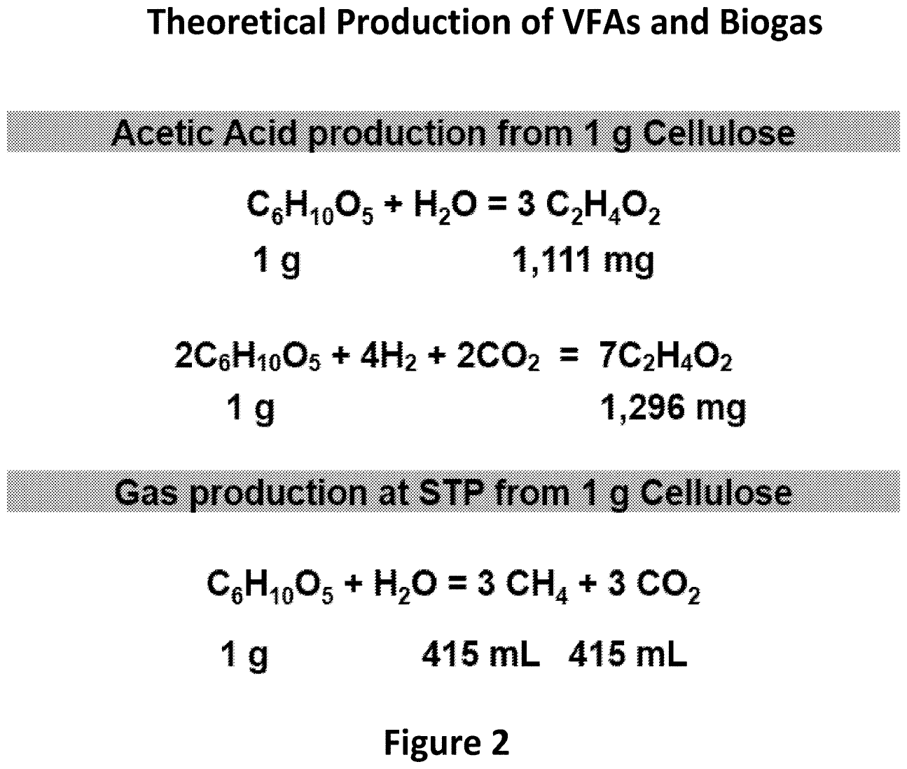

Cellulose and guar gum contain polysaccharides comprised of linked sugar monomers. Celluloytic organisms break down the linkages between monomers to convert them back into monomers, such as D-glucose. Once glucose becomes available, its aerobic or anaerobic degradation/fermentation produces VFAs as intermediate products which can then be converted to biogas. Thus, carbon-based compounds of this disclosure include VFAs and biogas. In certain embodiments, the principle components of VFA production include: liquefication; hydrolysis; solubilization; and fermentation, resulting in the production of organic acids, VFAs, H₂, CO₂, NH₃, and alcohols. Representative examples of VFAs produced by the methods of this disclosure include formic acid (HCOOH), acetic acid (CH₃COOH), propionic acid (C₂H₅COOH), and butyric acid (C₃H₇CHOOH). Additional representative examples of VFAs include those listed elsewhere herein. In certain embodiments, fermentation of cellulosic feedstocks produces acetic, propionic, iso-butyric and/or n-butyric acids. Theoretical acetic acid production (being the predominant component of VFAs) from 1 g of cellulose can be anywhere from 1.1 to 1.3 g, depending upon the metabolic pathways that they undergo (FIG. 2). The production/presence of VFAs can be determined by gas chromatography using known protocols. In certain embodiments, degradation is allowed to continue such that VFAs are converted into biogas.

### Temperature

Without being bound by theory, it is believed that up to a point, the reaction rate of cellulosic feedstock fermentation increases with an increase in temperature due to increased metabolic activity of cellulolytic organisms. In certain embodiments, fermentation of any of the cellulosic feedstocks disclosed herein under either aerobic or anaerobic conditions, and with or without mixing/agitation, and at any pH disclosed herein, can be performed at a temperature of any of between about 15° C., 20° C., 25° C., 30° C., or 35° C. and any of about 20° C., 25° C., 30° C., 35° C., or 40° C. In certain embodiments, such fermentation can be performed at a temperature of between about 30° C. and 35° C. In certain embodiments, such fermentation can be performed at a temperature of about 25° C., 26° C., 27° C., 28° C., 29° C., 30° C., 31° C., 32° C., 33° C., 34° C., 35° C., 36° C., or 37° C.

### Loading

For purpose of this disclosure, “loading rate” can be defined by the weight (e.g., dry weight, either determined or estimated) of feedstock per unit liquid feed volume (of sludge draw, nutrient solution, and/or plant effluent) fed to the reactor. Feedstock in grams can be viewed as loading per every 100 mL liquid feed after multiplying by 10. For example, 0.5 g guar gum per 100 mL liquid feed represents a loading rate of 5 g/L.

In certain embodiments of a fermentation process disclosed anywhere herein, any of the feedstocks disclosed herein can be loaded (i.e., have a loading rate) at between any of about 1 g/L, 2 g/L, 3 g/L, 4 g/L, 5 g/L, 6 g/L, 7 g/L, 8 g/L, 9 g/L, 10 g/L, 15 g/L 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, or 95 g/L to any of about 2 g/L, 3 g/L, 4 g/L, 5 g/L, 6 g/L, 7 g/L, 8 g/L, 9 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, or 100 g/L under either aerobic or anaerobic conditions. In certain embodiments, any of the feedstocks disclosed herein is loaded at about 1 g/L, 2 g/L, 3 g/L, 4 g/L, 5 g/L, 6 g/L, 7 g/L, 8 g/L, 9 g/L, 10 g/L, 15 g/L 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, or 100 g/L under either aerobic or anaerobic conditions.

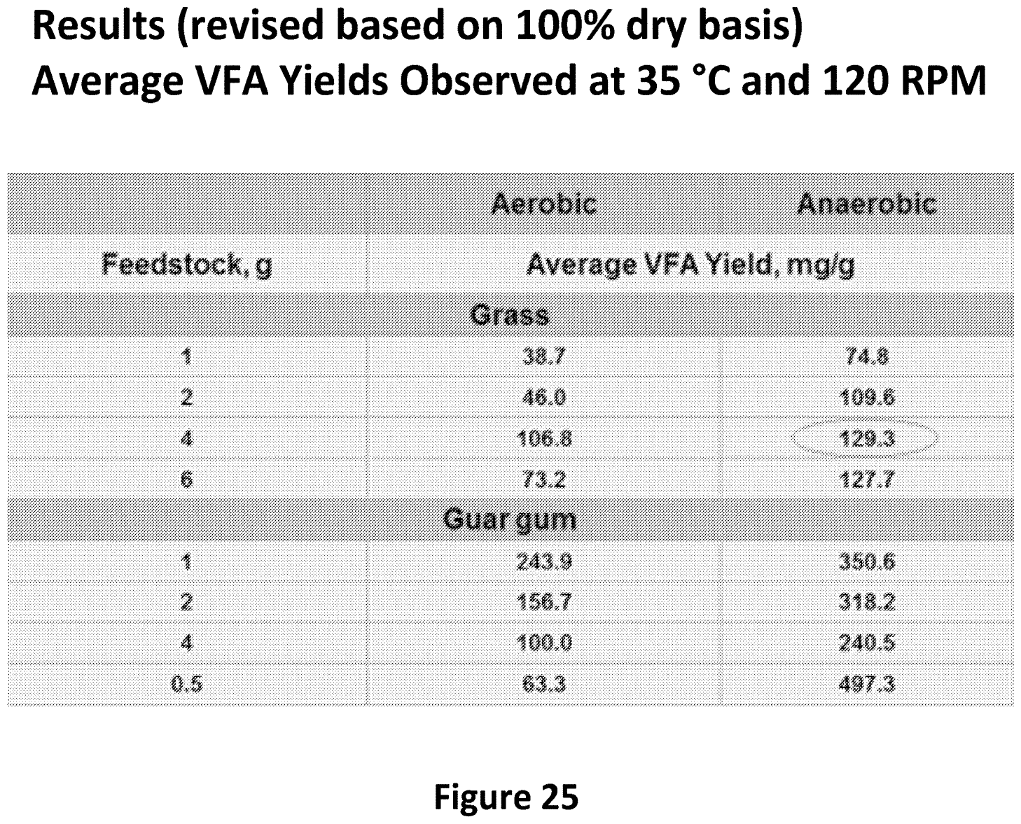

In certain embodiments of a fermentation process disclosed anywhere herein, grass feedstock can be loaded (i.e., have a loading rate) at between any of about 5 g/L, 10 g/L, 20 g/L, 30 g/L, 40 g/L, 50 g/L, or 60 g/L to any of about 10 g/L, 20 g/L, 30 g/L, 40 g/L, 50 g/L, 60 g/L, or 75 g/L under either aerobic or anaerobic conditions. In certain embodiments, grass feedstock is loaded at about 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L under either aerobic or anaerobic conditions. In certain embodiments, the grass feedstock can be loaded at about 40 g/L and in certain embodiments, such loading results in an average VFA production yield of at least about 106.8 mg/g per unit feedstock of grass under aerobic conditions (FIG. 25). In certain embodiments, the grass feedstock can be loaded at about 40 g/L and in certain embodiments, such loading results in an average VFA production yield of at least about 129.3 mg/g per unit feedstock of grass under anaerobic conditions (FIG. 25).

In certain embodiments of a fermentation process disclosed anywhere herein, guar gum feedstock can be loaded at between any of about 1 g/L, 2 g/L, 3 g/L 4 g/L, 5 g/L, 10 g/L, 20 g/L, 30 g/L, 40 g/L, 45 g/L, or 50 g/L to any of about 2 g/L, 3 g/L 4 g/L, 5 g/L, 10 g/L, 20 g/L, 30 g/L, 40 g/L, 50 g/L, or 60 g/L under either aerobic or anaerobic conditions. In certain embodiments, guar gum feedstock is loaded at about 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or 20 g/L under either aerobic or anaerobic conditions. In certain embodiments of a fermentation process disclosed anywhere herein, guar gum feedstock can be loaded at between any of about 5 g/L, 6 g/L, 7 g/L, 8 g/L, 9 g/L, 10 g/L, 11 g/L, 12 g/L, 13 g/L, or 14 g/L to any of about 6 g/L, 7 g/L 8 g/L, 9 g/L, 10 g/L, 11 g/L, 12 g/L, 13 g/L, 14 g/L, or 15 g/L under aerobic conditions. In certain embodiments, guar gum feedstock is loaded at about 5 g/L, 6 g/L, 7 g/L, 8 g/L, 9 g/L, 10 g/L, 11 g/L, 12 g/L, 13 g/L, 14 g/L, or 15 g/L under aerobic conditions. In certain embodiments, the guar gum feedstock can be loaded at 10 g/L and in certain embodiments, such loading results in an average VFA production yield of at least about 243.9 mg/g per unit feedstock of guar gum under aerobic conditions (FIG. 25). In certain embodiments of a fermentation process disclosed anywhere herein, guar gum feedstock can be loaded at between any of about 1 g/L, 2 g/L, 3 g/L, 4 g/L, 5 g/L, 6 g/L, 7 g/L, 8 g/L, or 9 g/L to any of about 2 g/L, 3 g/L, 4 g/L, 5 g/L, 6 g/L, 7 g/L, 8 g/L, 9 g/L, or 10 g/L under anaerobic conditions. In certain embodiments, guar gum feedstock is loaded at about 1 g/L, 2 g/L, 3 g/L, 4 g/L, 5 g/L, 6 g/L, 7 g/L, 8 g/L, 9 g/L, or 10 g/L under anaerobic conditions. In certain embodiments, the guar gum feedstock can be loaded at 5 g/L and in certain embodiments, such loading results in an average VFA production yield of at least about 497.3 mg/g per unit feedstock of guar gum under anaerobic conditions (FIG. 25).

pH

The optimum enzymatic activity of acidogens (acid formers) occurs within the pH range of about 5.0 to 6.2, whereas that of methanogens occurs within the pH range of about 6.8 to 7.2. Additionally, alkalinity addition through nutrient solution introduction may be omitted/minimized to keep the pH values suppressed to inhibit methanogens. The VFAs or volatile acids/alkalinity ratios within 0.5 to slightly higher than 0.8 range can be used to keep the methanogens suppressed; this state of operation is known as “sour operation” due to accumulation of VFAs, which tends to drop the pH.

Provided in this disclosure are two sets of pH parameters: (i) in the stock cultures from where inoculum is derived and (ii) inside the fermenting reactor. In certain embodiments, the pH values during stock culture maintenance and/or in fermenters during production can be in the range of between any of about pH 4.5, 4.6, 4.7, 4.8, 4.9, 5.0, 5.1, 5.2, 5.3, 5.4, 5.5, 5.5, 5.6, 5.7, 5.8, 5.9, 6.0, 6.1, 6.2, 6.3, or 6.4 to any of about pH 4.6, 4.7, 4.8, 4.9, 5.0, 5.1, 5.2, 5.3, 5.4, 5.5, 5.5, 5.6, 5.7, 5.8, 5.9, 6.0, 6.1, 6.2, 6.3, 6.4, or 6.5. In certain embodiments, the pH values during stock culture maintenance and/or in fermenters during production is about pH 4.5, 4.6, 4.7, 4.8, 4.9, 5.0, 5.1, 5.2, 5.3, 5.4, 5.5, 5.5, 5.6, 5.7, 5.8, 5.9, 6.0, 6.1, 6.2, 6.3, 6.4, or 6.5. In certain embodiments, the pH values during stock culture maintenance and/or in fermenters during production can be in the range of between any of about pH 5.0, 5.1, 5.2, 5.3, 5.4, 5.5, 5.5, 5.6, 5.7, 5.8, or 5.9 to any of about pH 5.1, 5.2, 5.3, 5.4, 5.5, 5.5, 5.6, 5.7, 5.8, 5.9, or 6.0. In certain embodiments, the pH values during stock culture maintenance and/or in fermenters during production is about pH 5.0, 5.1, 5.2, 5.3, 5.4, 5.5, 5.5, 5.6, 5.7, 5.8, 5.9. or 6.0. In certain embodiments, the pH values during stock culture maintenance and/or in fermenters during production does not exceed about pH 6.0, 6.1, 6.2, or 6.3. In certain embodiments, the pH values during stock culture maintenance and/or in fermenters during production does not exceed about pH 6.2. In certain embodiments, the pH values during stock culture maintenance and/or in fermenters during production does not exceed about pH 6.0. Without being bound by theory, the maintenance of pH according to the above helps to cultivate, promote, and maintain a maximum population of cellulolytic organisms while controlling methanogenic populations in check. VFAs are intermediate products of fermentation and are converted to biogas by methanogenic populations. Methanogens are very sensitive to acidic pH and enzymatic activity at or below 6.2 is greatly reduced or eliminated. Controlling the pH allows retention and harnessing of most of the VFAs produced, rather than being converted into biogas.

On the other hand, in certain embodiments, a pH value above 6.2, such as in the range of about pH 6.5 to 7.2 is useful for biogas production. In certain embodiments, a pH range of between any of about pH 6.2, 6.3, 6.4, 6.5, 6.5, 6.7, 6.8, 6.9, 7.0, 7.1, 7.2, 7.3, or 7.4 and any of about pH 6.3, 6.4, 6.5, 6.6, 6.7, 6.8, 6.9, 7.0, 7.1, 7.2, 7.3, 7.4, or 7.5 is used for biogas production. In certain embodiments, a pH range of between any of about pH 6.5, 6.5, 6.7, 6.8, 6.9, 7.0, or 7.1 and any of about pH 6.6, 6.7, 6.8, 6.9, 7.0, 7.1, or 7.2 is used for biogas production. In certain embodiments, a pH of about 6.3, 6.4, 6.5, 6.6, 6.7, 6.8, 6.9, 7.0, 7.1, 7.2, 7.3, 7.4, or 7.5 is used for biogas production. In certain embodiments, a pH of about 6.5, 6.6, 6.7, 6.8, 6.9, 7.0, 7.1, or 7.2 is used for biogas production.

### Timing

In certain embodiments it has been determined that methanogens consume VFAs if hydraulic retention time is greater than 120 hours. Thus, in certain embodiments, VFA harness occurs prior to 120 hours. In certain embodiments, VFA harness occurs after 48 hours or after 72 hours, for example, to allow ample time for VFA formation. In certain embodiments, VFA harness occurs between any of about 36 hours, 48 hours, 60 hours, 72 hours, 84 hours, 96 hours, or 108 hours to any of about 48 hours, 60 hours, 72 hours, 84 hours, 96 hours, 108 hours, or 120 hours. In certain embodiments, VFA harness occurs after about 48 hours, 60 hours, 72 hours, 84 hours, 96 hours, or 108 hours, but prior to 120 hours.

### Grass

Grass is a versatile plant that can be grown without maintenance and little water input. It is generally neglected in urban areas as it presents few beneficial uses upon mowing. It is mainly grown for beautification of site and increasing property values. Biodegradation of grass can occur aerobically and anaerobically. Without being bound by theory, every mole of cellulose can theoretically produce 3.00 to 3.50 moles of VFAs, depending upon the dominant degradation metabolic pathway.

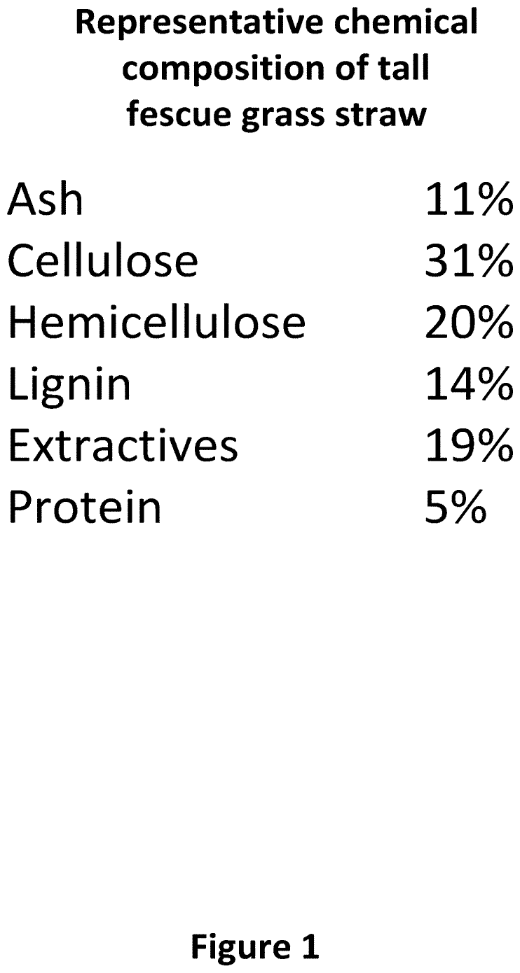

Thus, certain embodiments of this disclosure are drawn to the use of grass as a cellulosic feedstock and/or source of a cellulosic feedstock. Grasses belong to the Poaceae family, which is one of the most abundant families of plants on earth (world wide web at wiki.answers.com/Q/What_is_grass_made_of). While there are more than 10,000 varieties of plants in the Poaceae family (approximately 1,400 species of grasses exist in the United States), different types of grasses have some similarities. Grasses can be classified as either C3 or C4 plants. These terms refer to different pathways that plants use to capture carbon dioxide during photosynthesis. All species have the more primitive C3 pathway, but the additional C4 pathway evolved in species in the wet and dry tropics. All grasses produce seeds and are monocotyledonous. Additionally, most grasses are herbaceous, so they don't produce woody stems, and they die back to the ground at the end of the growing season. All grasses comprise water (generally about 70 to 80% by weight; turfgrass is about 75 to 80% water by weight) and lignin (world wide web at pennington.com/all-products/grass-seed/resources/10-surprising-facts-about-grass). FIG. 1 shows a representative chemical composition of a tall fescue grass straw (world wide web at wiki.answers.com/Q/What_is_grass_made_of; world wide web at answers.yahoo.com/question/index?qid=20060923151703AApc4vd; world wide web at en.wikipedia.org/wiki/Lignin; Impact of pretreatment and downstream processing technologies on economics and energy in cellulosic ethanol production, Deepak Kumar and Ganti S Murthy, Kumar and Murthy Biotechnology for Biofuels 2011, 4:27 http://www.biotechnologyforbiofuels.com/content/4/1/27). Lignin is a non-sugar polymer that crosslinks cellulose fibers together and comes in at least three different forms: C₉H₁₀O₂ (p-coumaryl alcohol); C₁₀H₁₂O₃ (coniferyl alcohol); and C₁₁H₁₄O₄ (sinapyl alcohol). The ratio between these units, the molecular weight, and the amount of lignin differs from plant to plant. As a rule of thumb, the amount of lignin generally decreases from softwoods to hardwoods to grasses. The typical lignin content is 24-33% in softwoods, 19-28% in hardwoods depending on the sources, and 15-25% in cereal straws, bamboo or bagasse.

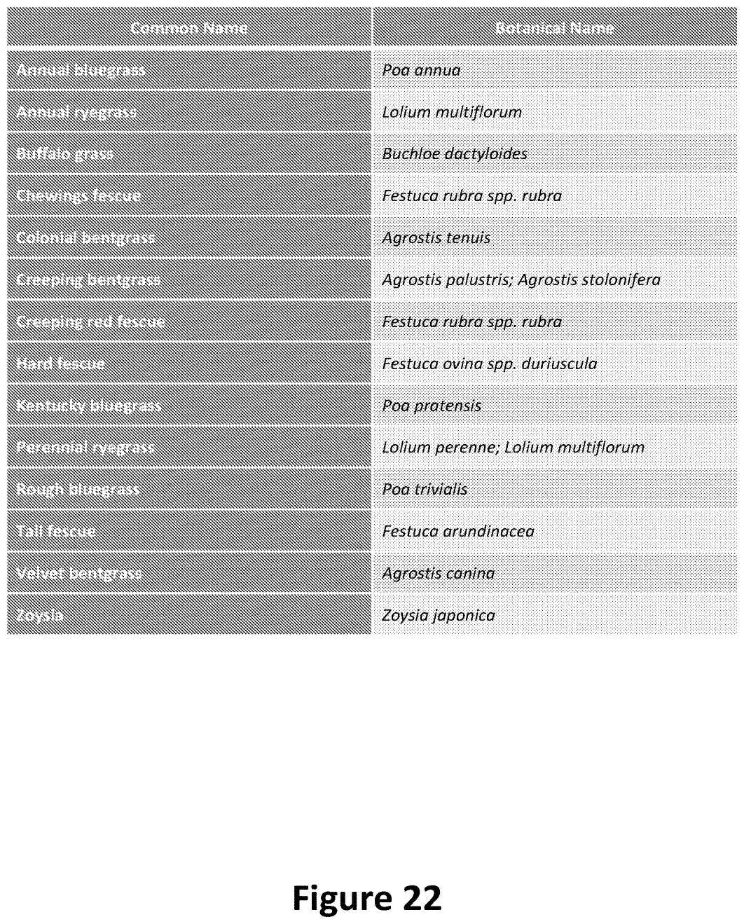

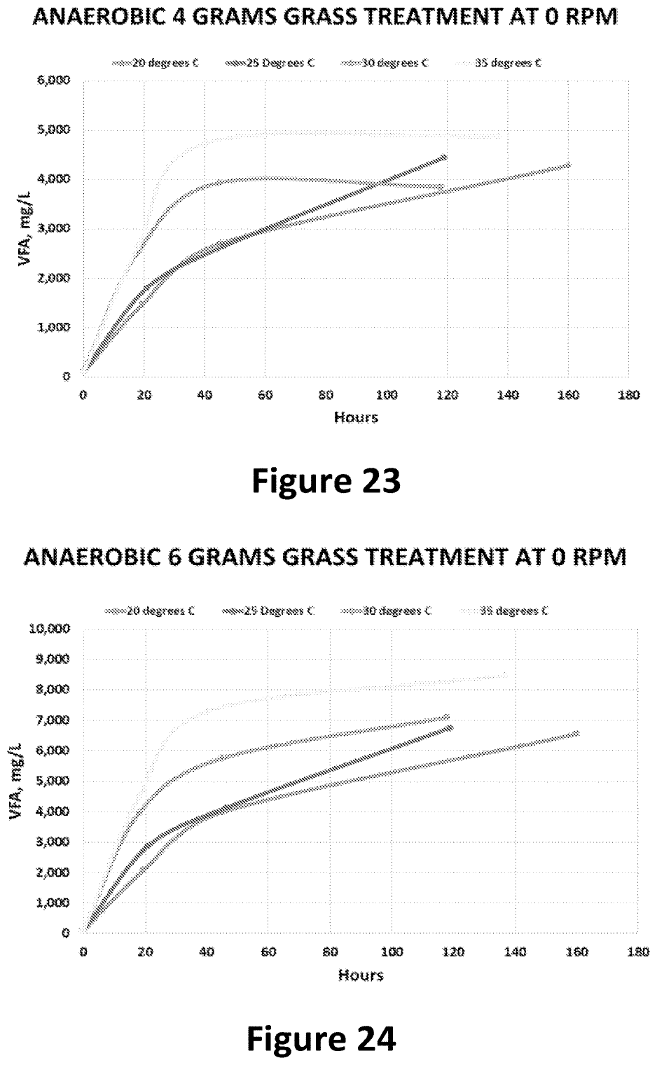

In certain embodiments, the grass used is a mixture of different grass families. In certain embodiments, the grass is from the genus “Poa” with comprising one or more of the subfamilies: Anomochlooideae; Aristidoideae; Arundinoideae; Bambusoideae; Chloridoideae; Danthonioideae; Ehrhartoideae; Micrairoideae; Paniocideae; Pharoideae; Pooideae; Puelioideae, of which there are several species in each subfamily. In certain embodiments, the grass contains one or more of Kentucky Bluegrass, Perennial Rye, Fescue (e.g., red fescue, hard fescue, sheep fescue, and tall fescue), Zoysia, Creeping Bent Grass, and/or one or more of the grasses listed in FIG. 22, or a mixture thereof of any of the preceding.

In certain embodiments, grass is used as a cellulosic feedstock and/or source of a cellulosic feedstock for fermentation under aerobic conditions and in in certain embodiments, grass is used as a cellulosic feedstock and/or source of a cellulosic feedstock for fermentation under anaerobic conditions. In certain embodiments, the VFA yield from grass under anaerobic conditions is better than the yield from primary sludge (from a typical municipal wastewater treatment plant) fermentation and high strength organic wastes (from industrial sources—brewery, fats, oil and grease processor, and chemical operations sludge with high organic content) co-fermentation with anaerobic digester sludge that receives a mixture of primary and secondary sludge.

In certain embodiments, the grass is dried or dried and pulverized. In certain embodiments, wet grass is reduced to its smallest practical size to minimize handling and operational problems (e.g., pumping and clogging). Additionally, wet or dry grass size reduction was for increasing surface area of substrate to cellulotytic organisms; increased surface area thus tends to achieve higher process efficiency. In certain embodiments, fermentation of grass was conducted at 35° C. In certain embodiments, the fermentation reaction is mixed or occasionally and/or intermittently mixed, for example at 120 RPM. In certain embodiments, the fermentation reaction is subjected to a detention time of about 24 hours, 36 hours, 48 hours, 60 hours, 72 hours, 96 hours, 108 hours, or up to 120 hours, or any specific time or time range in-between.

In certain embodiments, one gram of grass produces at least about 20 mg, 25 mg, 30 mg, 35 mg, 40 mg, 50 mg, 60 mg, 70 mg, 80 mg, 90 mg, 100 mg, 125 mg, 150 mg, 175 mg, 200 mg, 250 mg, 300 mg, 400 mg, 500 mg, or 750 mg of VFAs when fermented under certain aerobic conditions. In certain embodiments, one gram of grass produces at least about 30 mg, 35 mg, 40 mg, 50 mg, 60 mg, 70 mg, 80 mg, 90 mg, or 100 mg of VFAs when fermented under certain aerobic conditions. In certain embodiments, one gram of grass produces between any of about 20 mg, 25 mg, 30 mg, 35 mg, 40 mg, 50 mg, 60 mg, 70 mg, 80 mg, 90 mg, 100 mg, 125 mg, 150 mg, 175 mg, 200 mg, 250 mg, 300 mg, 400 mg, or 500 mg to any of about 25 mg, 30 mg, 35 mg, 40 mg, 50 mg, 60 mg, 70 mg, 80 mg, 90 mg, 100 mg, 125 mg, 150 mg, 175 mg, 200 mg, 250 mg, 300 mg, 400 mg, 500 mg, or 750 mg of VFAs when fermented certain under aerobic conditions. In certain embodiments, one gram of grass produces between any of about 35 mg, 40 mg, 50 mg, 60 mg, 70 mg, 80 mg, 90 mg, or 100 mg to any of about 40 mg, 50 mg, 60 mg, 70 mg, 80 mg, 90 mg, 100 mg, 110 mg, or 125 mg of VFAs when fermented under certain aerobic conditions.

In certain embodiments, one gram of grass produces at least about 20 mg, 25 mg, 30 mg, 35 mg, 40 mg, 50 mg, 60 mg, 70 mg, 80 mg, 90 mg, 100 mg, 125 mg, 150 mg, 175 mg, 200 mg, 250 mg, 300 mg, 400 mg, 500 mg, or 800 mg of VFAs when fermented under certain anaerobic conditions. In certain embodiments, one gram of grass produces at least about 60 mg, 70 mg, 80 mg, 90 mg, 100 mg, 125 mg, 150 mg, 175 mg, or 200 mg of VFAs when fermented under certain anaerobic conditions. In certain embodiments, one gram of grass produces between any of about 40 mg, 50 mg, 60 mg, 70 mg, 80 mg, 90 mg, 100 mg, 125 mg, 150 mg, 175 mg, 200 mg, 250 mg, 300 mg, 400 mg, or 500 mg to any of about 50 mg, 60 mg, 70 mg, 80 mg, 90 mg, 100 mg, 125 mg, 150 mg, 175 mg, 200 mg, 250 mg, 300 mg, 400 mg, 500 mg, or 800 mg of VFAs when fermented under certain anaerobic conditions. In certain embodiments, one gram of grass produces between any of about 60 mg, 70 mg, 80 mg, 90 mg, 100 mg, 125 mg, 150 mg, 175 mg, or 200 mg to any of about 70 mg, 80 mg, 90 mg, 100 mg, 125 mg, 150 mg, 175 mg, 200 mg, or 250 mg of VFAs when fermented under certain anaerobic conditions.

### Guar Gum

Certain embodiments are drawn to the use of guar gum (also known as guaran) as a cellulosic feedstock and/or source of a cellulosic feedstock. Guar gum (e.g., CAS Number: 9000-30-0), is a substance made from guar beans which has thickening and stabilizing properties useful in various industries; e.g., traditionally the food industry and, increasingly, the hydraulic fracturing industry. Guar seeds are dehusked, milled and screened to obtain the guar gum in powder form. Chemically, guar gum is a polysaccharide composed of the sugars galactose and mannose (classified as a galactomannan). It is typically produced as a free-flowing, off-white powder. In water, it is nonionic and hydrocolloidal. Guar gum is also sometimes referred to in this disclosure as plant extract (PE).

In certain embodiments, guar gum is used as a cellulosic feedstock and/or source of a cellulosic feedstock for fermentation under aerobic conditions and in in certain embodiments, guar gum is used as a cellulosic feedstock and/or source of a cellulosic feedstock for fermentation under aerobic conditions.

In certain embodiments, fermentation of guar gum was conducted at 35° C. In certain embodiments, the fermentation reaction is mixed or occasionally and/or intermittently mixed, for example at 120 RPM. In certain embodiments, the fermentation reaction is subjected to a detention time of about 24 hours, 36 hours, 48 hours, 60 hours, 72 hours, 96 hours, 108 hours, or up to 120 hours, or any specific time or time range in-between.

In certain embodiments, one gram of guar gum produces at least about 40 mg, 50 mg, 60 mg, 70 mg, 80 mg, 90 mg, 100 mg, 125 mg, 150 mg, 175 mg, 200 mg, 250 mg, 300 mg, 400 mg, 500 mg, 750 mg, 900 mg, or 1000 mg of VFAs when fermented under aerobic conditions. In certain embodiments, one gram of guar gum produces at least about 40 mg, 50 mg, 60 mg, 70 mg, 80 mg, 90 mg, 100 mg, 125 mg, 150 mg, 175 mg, 200 mg, 250 mg, or 300 mg of VFAs when fermented under aerobic conditions. In certain embodiments, one gram of guar gum produces between any of about 40 mg, 50 mg, 60 mg, 70 mg, 80 mg, 90 mg, 100 mg, 125 mg, 150 mg, 175 mg, 200 mg, 250 mg, 300 mg, 400 mg, 500 mg, 750 mg, or 900 mg to any of about 50 mg, 60 mg, 70 mg, 80 mg, 90 mg, 100 mg, 125 mg, 150 mg, 175 mg, 200 mg, 250 mg, 300 mg, 400 mg, 500 mg, 750 mg, 900 mg, or 1000 mg of VFAs when fermented under aerobic conditions. In certain embodiments, one gram of guar gum produces between any of about 50 mg, 60 mg, 70 mg, 80 mg, 90 mg, 100 mg, 125 mg, 150 mg, 175 mg, 200 mg, or 250 mg to any of about 60 mg, 70 mg, 80 mg, 90 mg, 100 mg, 125 mg, 150 mg, 175 mg, 200 mg, 250 mg, or 300 mg of VFAs when fermented under aerobic conditions.

In certain embodiments, one gram of guar gum produces at least about 50 mg, 100 mg, 125 mg, 150 mg, 175 mg, 200 mg, 250 mg, 300 mg, 400 mg, 500 mg, 750 mg, 900 mg, or 1000 mg of VFAs when fermented under anaerobic conditions. In certain embodiments, one gram of guar gum produces at least about 150 mg, 175 mg, 200 mg, 250 mg, 300 mg, 350 mg, 400 mg, 500 mg, or 750 mg of VFAs when fermented under anaerobic conditions. In certain embodiments, one gram of guar gum produces between any of about 100 mg, 150 mg, 175 mg, 200 mg, 250 mg, 300 mg, 400 mg, 500 mg, 750 mg, or 900 mg to any of about 150 mg, 175 mg, 200 mg, 250 mg, 300 mg, 400 mg, 500 mg, 750 mg, 900 mg, or 1000 mg of VFAs when fermented under anaerobic conditions. In certain embodiments, one gram of guar gum produces between any of about 175 mg, 200 mg, 250 mg, 300 mg, 350 mg, 400 mg, 450 mg, or 500 mg to any of about 200 mg, 250 mg, 300 mg, 350 mg, 400 mg, 450 mg, 500 mg, or 600 mg of VFAs when fermented under anaerobic conditions.

### Cattail

Cattails are an aquatic plant common to ponds, marshes, lakes, and swamps, and are very easy to identify. They are distinguished by the unique seed heads on top of the stalks, which go from a hot dog or cigar-looking shape to a fluffy mass that looks somewhat like a cat's tail.

Cattails emerge from the muck in a long, round stalk that stands up to six feet or higher above the water. Leaves are mostly flat, narrow, and lance-shaped, emerging from the stalk in an alternating pattern. The only real lookalikes are different varieties of the Iris plant, and it's only the leaves that look similar. Iris and cattail are easy to differentiate, from the distinctive seed heads (or any remnant of them, if they're out of season); with true cattails, the seed head is always left behind.

In certain embodiments, cattails are used as a cellulosic feedstock and/or source of a cellulosic feedstock for fermentation under aerobic conditions as described for grass and/or guar gum anywhere else in this disclosure and in certain embodiments, cattails are used as a cellulosic feedstock and/or source of a cellulosic feedstock for fermentation under aerobic conditions as described for grass and/or guar gum anywhere else in this disclosure. In certain embodiments, the cattail used is one or more species of cattail listed in Table 1, or a mixture thereof. In certain embodiments, a cattail used is the cattail known as common cattail or broadleaf cattail, i.e., Typha Latifolia.

TABLE 1
broadleaf cattail - Species: Typha latifolia
cattail gayfeather - Variety: Liatris pycnostachya var. lasiophylla Skinners
cattail gayfeather - Variety: Liatris pycnostachya var. pycnostachya
cattail grass - Species: Setaria pumila
cattail sedge - Species: Carex typhina
common cattail - Species: Typha latifolia
narrowleaf cattail - Species: Typha angustifolia
southern cattail - Species: Typha domingensis
white cattail - Species: Typha × glauca Godr.

### Phragmites

Phragmites australis, or common reed, is also known by many synonyms including: Phragmites; Arundo altissima; Arundo australis; Arundo graeca; Arundo isiaca; Arundo maxima; Arundo occidentalis; Arundo palustris; Arundo phragmites; Arundo vulgaris; Cynodon phragmites; Oxyanthe phragmites; Phragmites latissimus; Phragmites capensis; Phragmites caudatus; Phragmites chilensis; Phragmites dioicus; Phragmites fissifolius; Phragmites hispanicus; Phragmites isiacus; Phragmites martinicensis; Phragmites mauritianus; Phragmites maximus; Phragmites occidentalis; Phragmites; Phragmites vulgaris; Reimaria diffusa; Trichoon phragmites; Phragmites australis ssp. Maximus; Phragmites communis ssp. Maximus; Phragmites vulgaris ssp. Maximus; Phragmites communis var. flavescens; Phragmites communis var. genuinus; Phragmites communis var. hispanicus; Phragmites communis var. isiacus; Phragmites communis var. mauritianus; Phragmites communis var. variegatus; Phragmites maximus var. variegatus; Phragmites vulgaris var. mauritianus.

In certain embodiments, phragmites are used as a cellulosic feedstock and/or source of a cellulosic feedstock for fermentation under anaerobic conditions as described for grass and/or guar gum anywhere else in this disclosure and in certain embodiments, phragmites are used as a cellulosic feedstock and/or source of a cellulosic feedstock for fermentation under anaerobic conditions as described for grass and/or guar gum anywhere else in this disclosure.

### Cellulolytic Organisms

Fermentation is a microbial process that relies on the presence of many different types of microbes to work as a consortium in harmony as complimentary supplementary relationships. The microbial community that makes up a specific fermenter's microbiome (the combination of all types of microbes in the fermenter) will be influenced by operational and design conditions, such as substrate type (feedstocks), biomass, hydraulic retention time, mixing, operating temperature, pH, and chemical addition (buffer solution or nutrient solution or foam prevention solution). The microbial community in turn will affect fermenter outcomes such as COD (chemical oxygen demand, a measure of carbon strength of the feed) removal and VFAs and biogas production.

Provided herein are the identities of certain bacterial and non-bacterial species responsible for cellulosic biomass degradation. Samples from grass aerobic, grass anaerobic, guar gum aerobic, and guar gum anaerobic stock cultures were analyzed by 16S amplicon sequencing technique and by metagenomics shotgun technique. The 16S amplicon sequencing data analysis was used to examine the celluloytic microbial community (bacterial species only) in four stock culture samples. This method provides taxonomic annotation and evidence of the presence of the functional gene; however, this technique can't identify organisms down to species level. It may be possible to infer functional genes from the 16S. It is not possible to detect any fungi or other types of organisms from the sample, as the 16S gene is only present in bacteria. It is theoretically possible to detect archaea using the 16S primers; however, the typical primers are more specific to bacteria and the bacterial sequence data would likely overshadow or obscure any archaeal sequences that are present. Identification of organisms is generally according to the nomenclature rules and are identified in the order, such as Super Kingdom, Kingdom, Phylum, Division or Class, Order, Family, Tribe, Genus, and Species.

A shotgun metagenomics approach can provide taxonomic alignments/annotations and detect the actual functional genes that are present in the samples as well as organisms from all domains of life, e.g. bacteria, archaea, eukaryotes, fungi, yeast, and viruses etc. There are taxonomic summaries for kingdom through species level. This technique is less sensitive with respect to 16S amplicon sequencing. Although shotgun metagenomic methods are not always as sensitive as 16S amplicon sequencing they can make up for this deficiency in the breadth of organisms and genes that can be detected

As determined in the non-limiting Examples below, certain results indicated that four sample conditions are quite similar to each other, particularly in terms of the functional gene annotation level. By taxonomic annotation, samples from aerobic conditions with grass as the feedstock and anaerobic conditions with grass as a feedstock are more similar to each other while samples from aerobic conditions with guar gum as a feedstock and anaerobic conditions with guar gum as a feedstock are more similar to each other. Nonetheless, all sample outcomes were similar at about an 80% level.

Known cellulolytic bacterial species (including both aerobic and anaerobic species) include: Micrococcus spp., Bacillus spp., Pseudomonas spp., Xanthomonas spp., Acetobacter Xylinum, and Brucella spp. Known cellulolytic fungal strains (including both aerobic and anaerobic strains) include: Chaetomium, Fusarium Myrothecium, Trichoderma. Penicillium, Aspergillus, and so forth, are some of the fungal species responsible for cellulosic biomass hydrolyzation. Other bacterial species include Trichonympha, Clostridium, Actinomycetes, Bacteroides succinogenes, Butyrivibrio fibrisolvens, Ruminococcus albus, and Methanobrevibacter ruminantium.

In certain embodiments, cellulolytic organisms were identified with 16S amplicon DNA technology. Organisms were identified for the degradation of grass and guar gum under both aerobic and anaerobic fermentation conditions. It was observed that the samples were highly diverse at the “species” level with roughly 500 taxa observed per sample. One of the most abundant bacterial organisms identified in all four reactors as a taxon was:
D_0_Bacteria; D_1_Chloroflexi; D_2_Anaerolineae; D_3_Anaerolineales; D_4_Anaerolineaceae.

Under guar gum anaerobic conditions, another highly abundant bacterial organism was:
D_0_Bacteria; D_1_Bacteroidetes; D_2_Bacteroidia; D_3_Bacteroidales; D_4_Bacteroidaceae; D_5 Bacteroides.

### Biogas

Anaerobic operations are catabolic/destructive processes that occur in the absence of free molecular oxygen while aerobic processes occur in the presence of free and molecular oxygen. If the inoculum is not adequately controlled for methanogens, then VFAs (the intermediate products of fermentation) are converted into an end product herein referred to as “biogas.” In certain embodiments, biogas is an end product of fermentation of celluloytic biomass, such as grass, guar gum, under aerobic and anaerobic operating conditions, and leaves, phargmites, and cattails under anaerobic operating conditions and is a biological conversion of such biomass into gaseous forms, predominantly comprising of methane and carbon dioxide. The percentage proportions and percentage proportions of trace organic and inorganic gases vary from feedstock to feedstock and operating conditions of fermenters. In certain embodiments, other trace gases can be carbon monoxide, carbon disulfide, hydrogen, nitrogen, nitrous oxide (N₂O), ammonia (NH₃), hydrogen sulfide (H₂S) and other odorous gases such as mercaptans (if sulfur is contained in feedstocks or comes with them as an impurity). Aerobic operations will predominantly produce carbon dioxide and water.

### Nutrient Solution.

In certain embodiments, a nutrient solution can be added to culture reactors to aid in maintaining cellulolytic culture for VFAs and biogas production. A non-limiting, representative nutrient solution formulation is shown in Table 9.

TABLE 9
Representative Salt-Vitamin Solution Recipe.

| Chemical Formula | Concentration (mg/L) |
|---|---|
| NH₄HCO₃ | 3,000 |
| NaCl | 900 |
| K₂HPO₄ | 900 |
| (NH₄)₂SO₄ | 450 |
| Na₂CO₃ | 320 |
| MgSO₄•7H₂O | 210 |
| CaCl₂•2H₂O | 120 |
| FeSO₄•7H₂O | 21 |
| MnSO₄•2H₂O | 5 |
| CoCl₂•6H₂O | 1 |
| ZnSO₄•7H₂O | 1 |
| CuSO₄•5H₂O | 0.1 |
| AlK(SO₄)₂•12H₂O | 0.1 |
| H₃BO₃ | 0.1 |
| Na₂MoO₄•2H₂O | 0.1 |
| Pyridoxine HCl, B6 | 0.1 |
| Thiamine HCl, B1 | 0.05 |
| Riboflavin, B2 | 0.05 |
| Nicotinic acid, niacin | 0.05 |
| Biotin | 0.02 |
| Folic acid | 0.02 |
| B12 crystalline | 0.005 |

In certain embodiments, a nutrient solution is not added if only VFAs production is desired, because such nutrient solutions contain buffering chemicals and that promote or harbor methanogens. The addition of a nutrient solution in stock culture reactors provides trace elements and vital vitamins and can help maintain all cellulolytic organisms in an equal functional state. Derivation of inoculum from stock cultures can result in less variability and more reproducibility due to consistency of the strength of organisms.

In certain embodiments, the addition of a nutrient solution is used to increase the rate of initial VFA production in very early period of the fermentation process. Such increased production rate can cut down the size of fermenting reactor volume. In certain embodiments, the addition of a nutrient solution is used to speed recovery of the mass of cellulolytic organisms after the process upsets, for example due to toxic substances.

### Wastewater Treatment.

Carbon-based compounds such as VFAs of the present disclosure can be used in wastewater treatment operations and other industrial settings where soluble carbon is needed for their operations.

The methods of producing carbon-based compounds of this disclosure are well-suited, for example, for wastewater treatment operations, where at least one or more of the following conditions prevail: i) the raw sewage has limited carbon in combined sewerage system due to inclusion of storm water; ii) limited carbon in the raw sewage is diluted with rainfall runoff and other drainage ending up in the Tunnel and Reservoir Plan (TARP) flows; iii) phosphorous is biologically removed but has an unattractive ratio of carbon to P; iv) the limited carbon is available to achieve multiple goals, such as biogas production and other carbon dependent liquid stream and resource recovery operations like enhanced biological phosphorus removal (EBPR), dentrification, and waste activated sludge stripping to recover internal phosphate (WASSRIP®); and v) external carbon is purchased to sustain the above mentioned process performance.

Disclosed herein are methods of producing a volatile fatty acid by fermenting a cellulosic feedstock present in a fermentation solution. Descriptions of various feedstocks are provided in detail elsewhere herein. In certain embodiments, the fermentation solution comprises cellulolytic microorganisms and the cellulosic feedstock. Descriptions of cellulotytic microorganisms are provided in detail elsewhere herein. In certain embodiments, the cellulosic feedstock is grass, leaves, phragmites, cattails, guar gum, or a combination of thereof. In certain embodiments, the cellulosic feedstock is grass or guar gum.

In certain embodiments, the cellulolytic microorganisms are supplied to the fermentation solution in an inoculum comprising the cellulolytic microorganisms. In certain embodiments, the inoculum can come from a seed source reservoir which can, for example, be a digester draw from anaerobic mesophilic digester in a municipal sewage treatment plant, such as disclosed in Example 6 of the non-limiting examples below. In certain embodiments, the fermentation solution comprises nutrients to support the viability of the cellulolytic microorganisms. In certain embodiments, the nutrients are supplied to the fermentation solution in a nutrient solution, a representative example of which is shown in Table 9.

In certain embodiments, the composition of identities of and/or the metabolic pathways utilized by the cellulolytic microorganisms supplied to the fermentation solution have been previously adapted or derived to the type of cellulosic feedstock and/or fermentation conditions to be used. “Adapted,” “adapting,” “adaptation,” and the like involves making the cellulolytic microorganisms use a certain feedstock and/or fermentation condition to steer their identities and metabolic pathways to meet their carbon and cellular energy needs such that the cellulolytic microorganisms are trained and adapted to derive their carbon and energy needs from the feedstock and/or fermentation condition. As used herein, reference to “derived,” “deriving,” and the like is used interchangeably for the same process and/or phase of the methods disclosed herein.

In certain embodiments, the cellulosic feedstock is fermented under aerobic conditions. In certain embodiments, the cellulosic feedstock is fermented under anaerobic conditions. In certain embodiments, the fermentation solution is agitated, either continually, periodically, intermittently, or the like, during fermentation. In certain embodiments, the agitation is achieved by stirring the fermentation solution within a fermentation vessel. In certain embodiments, the agitation occurs at 120 RPM.

In certain embodiments, the cellulosic feedstock is fermented at a temperature of between any of about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or 35° C. to any of about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In certain embodiments, the cellulosic feedstock is fermented at a temperature of between about 20° C. to 35° C.

The loading rate of the cellulosic feedstock is described in detail elsewhere herein. For example, in certain embodiments, the loading rate of the cellulosic feedstock is: between about 30 g/L to 50 g/L in the fermentation solution, wherein the feedstock is grass fermented under aerobic or anaerobic conditions; between about 5 g/L and 15 g/L in the fermentation solution, wherein the feedstock is guar gum fermented under aerobic conditions; or between about 1 g/L and 10 g/L in the fermentation solution, wherein the feed stock is guar gum fermented under anaerobic conditions.

The pH at which the cellulosic feedstock can be fermented is described in detail elsewhere herein. For example, in certain embodiments, the cellulosic feedstock is fermented at a pH of less than about pH 6.2 or between about pH 5.0 and 6.0 if VFA production is desired. For example, in certain embodiments, the cellulosic feedstock is fermented at a pH of above about pH 6.3 or between about pH 6.5 and pH 7.2 if biogas production is desired.

In certain embodiments, at least one VFA produced is selected from the group consisting of acetic acid, propionic acid, iso-butyric acid, and N-butyric acid. In certain embodiments, at least two, at least three, or at least four VFAs produced are selected from the group consisting of acetic acid, propionic acid, iso-butyric acid, and N-butyric acid. In certain embodiments, the fermentation of the cellulosic feedstock proceeds to further generate a biogas as described in detail elsewhere herein. For example, in certain embodiments, the biogas produced is methane and/or carbon dioxide.

In certain embodiments disclosed anywhere herein, the cellulosic feedstock is grass which has been dried and pulverized before incorporation into the fermentation solution. And, in certain embodiments, the grass is not switchgrass.

In certain embodiments, cellulase is added to the fermentation solution. While cellulase is not necessary, and may not necessarily improve overall yield, it can be used to increase the initial reaction rate.

### VFAs

A fatty acid is a carboxylic acid with a long aliphatic chain, which can be saturated or unsaturated. Most naturally occurring fatty acids have an unbranched chain of an even number of carbon atoms, from 4 to 28. Fatty acids differ by length, often categorized as short to very long. Short-chain fatty acids (SCFA) are generally considered fatty acids with aliphatic tails of five or fewer carbons (e.g. butyric acid). Medium-chain fatty acids (MCFA) are generally considered fatty acids with aliphatic tails of 6 to 12 carbons, which can form medium-chain triglycerides. Long-chain fatty acids (LCFA) are generally considered fatty acids with aliphatic tails of 13 to 21 carbons. And, very long chain fatty acids (VLCFA) are generally considered fatty acids with aliphatic tails of 22 or more carbons. Provided herein are some of the most common forms of low-molecular-weight VFAs of importance with additional acids names in the table below.

Short-chain fatty acids (SCFAs). Some common forms of short chain free VFAs include: Formic acid; Acetic acid; Propionic acid; Isobutyric acid; Butyric acid; Isovaleric acid; and Valeric acid.

Medium-chain fatty acids (MCFAs). Some common forms of medium chain VFAs include: Caproric acid (n-Hexanoic Acid); Enanthoic Acid (n-Heptanoic acid); Caprylic acid (n-Octanoic Acid); alpha-Ethylcaproic Acid (2-Ethylhexanoic Acid); Valproic Acid (2-Propylpentanoic Acid); Pelargonic Acid (n-Nonanoic Acid); Capric acid (n-Decanoic Acid); Undecylic acid (systematically named undecanoic acid); and Dodecylic acid (systematically named Dodecanoic acid; Lauric acid and Fulvic acid are common).

Long-chain fatty acids (LCFAs). LCFAS are found in most fats and oils, including olive oil, soybean oil, fish, nuts, avocado and meat. Some common forms include: Tridecylic acid; Tetradecanoic acid (Myristic acid); Pentadecylic acid; Hexadecanoic acid (Palmitic acid); Heptadecanoic acid (Margaric acid and Heptadecylic acid); Octadecanoic acid (Stearic acid); (9Z)-octadec-9-enoic acid (Oleic acid); (9Z,12Z)-octadeca-9,12-dienoic acid (Linoleic acid (with two double bonds); Linoleic acid (with three double bonds)); Nonadecylic acid; Eicosanoic acid (Arachidic acid; Arachic acid; Arachidonic acid; Mead's acid); and Heneicosanoic acid.

Very Long chain free volatile fatty acids (VLCFA). Some common forms include: Docosanoic acid (Behenic acid; DHA Cervonic acid); Tricosanoic acid (Tricosylic acid); Tetracosanoic acid (Lignoceric acid); Pentacosanoic acid (Pentacosylic acid); and Hexacosanoic acid (Cerotic acid).

Table 40 is list of carboxylic acids ordered by the number of carbon atoms in the carboxylic acid.

TABLE 40

| IUPAC name | Common name | Structural formula |
|---|---|---|
| methanoic acid | formic acid | HCOOH |
| ethanoic acid | acetic acid | CH₃COOH |
| ethanedioic acid | oxalic acid | HOOCCOOH |
| oxoethanoic acid | glyoxylic acid | OHCCOOH |
| | formylformic acid | |
| 2-hydroxyethanoic acid | glycolic acid | HOCH₂COOH |
| | dicarbonous acid | |
| | hydroxyacetic acid | |
| propanoic acid | propionic acid | CH₃CH₂COOH |
| | ethanecarboxylic acid | |
| prop-2-enoic acid | acrylic acid | CH₂═CHCOOH |
| | acroleic acid | |
| | ethylenecarboxylic acid | |
| | propene acid | |
| | vinylformic acid | |
| 2-propynoic acid | propiolic acid | CH≡CCOOH |
| | acetylene carboxylic acid | |
| | propargylic acid | |
| propanedioic acid | malonic acid | HOOCCH₂COOH |
| | methanedicarboxylic acid | |
| 2-hydroxypropanedioic acid | tartronic acid | HOOCCHOHCOOH |
| | hydroxymalonic acid | |
| oxopropanedioic acid | mesoxalic acid | HOOCCOCOOH |
| | ketomalonic acid | |
| 2,2-dihydroxypropanedioic | dihydroxymalonic acid | HOOCC(OH)₂COOH |
| acid | Mesoxalic acid monohydrate | |
| 2-oxopropanoic acid | pyruvic acid | CH₃COCOOH |
| | α-ketopropionic acid | |
| | acetylformic acid | |
| | pyroracemic acid | |
| 2-hydroxypropanoic acid | lactic acid | CH₃CHOHCOOH |
| | milk acid | |
| 3-hydroxypropanoic acid | hydracrylic acid | CH₂OHCH₂COOH |
| 2,3-dihydroxypropanoic acid | glyceric acid | CH₂OHCHOHCOOH |
| 2-oxiranecarboxylic acid | glycidic acid | epoxide |
| butanoic acid | butyric acid | CH₃(CH₂)₂COOH |
| | propanecarboxylic acid | |
| 2-methylpropanoic acid | isobutyric acid | (CH₃)₂CHCOOH |
| | isobutanoic acid | |
| 2-oxobutanoic acid | alpha-ketobutyric acid | CH₃CH₂COCOOH |
| 3-oxobutanoic acid | acetoacetic acid | CH₃COCH₂COOH |
| 4-oxobutanoic acid | succinic semialdehyde | COHCH₂CH₂COOH |
| (E)-butenedioic acid | fumaric acid | HOOCCH═CHCOOH |
| | trans-1,2-ethylenedicarboxylic | |
| | acid | |
| | 2-butenedioic acid | |
| | trans-butenedioic acid | |
| | allomaleic acid | |
| | boletic acid | |
| | donitic acid | |
| | lichenic acid | |
| (Z)-butenedioic acid | maleic acid | HOOCCH═CHCOOH |
| | cis-butenedioic acid | |
| | maleinic acid | |
| | toxilic acid | |
| But-2-ynedioic acid | acetylenedicarboxylic acid | HOOCC≡CCOOH |
| | butynedioic acid | |
| | but-2-ynedioic acid | |
| | 2-Butynedioic acid | |
| oxobutanedioic acid | oxaloacetic acid | HOOCCH₂COCOOH |
| | oxalacetic acid | |
| | oxosuccinic acid | |
| hydroxybutanedioic acid | malic acid | HOOCCH₂CHOHCOOH |
| | hydroxybutanedioic acid | |
| 2,3-dihydroxybutanedioic acid | tartaric acid | HOOC(CHOH)₂COOH |
| | 2,3-dihydroxysuccinic acid | |
| | threaric acid | |
| | racemic acid | |
| | uvic acid | |
| | paratartaric acid | |
| (E)-but-2-enoic acid | crotonic acid | CH₃CH═CHCOOH |
| | trans-2-butenoic acid | |
| | beta-methylacrylic acid | |
| | 3-methylacrylic acid | |
| | (E)-2-butenoic acid | |
| pentanoic acid | valeric acid | CH₃(CH₂)₃COOH |
| | valerianic acid | |
| | butane-1-carboxylic acid | |
| 3-methylbutanoic acid | Iso-valeric acid | (CH₃)₂CHCH₂CO2H |
| pentanedioic acid | glutaric acid | HOOC(CH2)₃COOH |
| | propane-1,3-dicarboxylic acid | |
| | 1,3-propanedicarboxylic acid | |
| | n-pyrotartaric acid | |
| 2-oxopentanedioic acid | alpha-ketoglutaric acid | HOOC(CH₂)₂COCOOH |
| | 2-ketoglutaric acid | |
| | α-ketoglutaric acid | |
| | 2-oxoglutaric acid | |
| | oxoglutaric acid | |
| 3-oxopentanedioic acid | acetonedicarboxylic acid | HOOC(CH₂)₂COCOOH |
| | 1,3-acetonedicarboxylic acid | |
| | 3-oxoglutaric acid | |
| | 3-ketoglutaric acid | |
| | β-ketoglutaric acid | |
| furan-2-carboxylic acid | 2-furoic acid | furan-COOH |
| | α-furoic acid | |
| | 2-carboxyfuran | |
| | α-furancarboxylic acid | |
| | pyromucic acid | |
| tetrahydro-2-furancarboxylic | tetrahydro-2-furoic acid | tetrahydrofuran-COOH |
| acid | tetrahydrofuran-2-carboxylic | |
| | acid | |
| | tetrahydrofuroic acid | |
| hexanoic acid | caproic acid | CH₃(CH₂)₄COOH |
| | n-caproic acid | |
| hexanedioic acid | adipic acid | HOOC(CH₂)₄COOH |
| | hexane-1,6-dioic acid | |
| 2-hydroxypropane-1,2,3- | citric acid | HOC(COOH)((CH₂)COOH)₂ |
| tricarboxylic acid | 3-carboxy-3- | |
| | hydroxypentanedioic acid | |
| | 2-hydroxy-1,2,3- | |
| | propanetricarboxylic acid | |
| prop-1-ene-1,2,3-tricarboxylic | aconitic acid | HOOCCH═C(COOH)CH₂COOH |
| acid | achilleic acid | |
| | equisetic acid | |
| | citridinic acid | |
| | pyrocitric acid | |
| 1-hydroxypropane-1,2,3- | isocitric acid | HOOCCHOHCH(COOH)CH₂COOH |
| tricarboxylic acid | | |
| (2E,4E)-hexa-2,4-dienoic acid | sorbic acid | CH₃(CH═CH)₂COOH |
| heptanoic acid | enanthic acid | CH₃(CH₂)₅COOH |
| | oenanthic acid | |
| | n-Heptylic acid | |
| | n-Heptoic acid | |
| heptanedioic acid | pimelic acid | HOOC(CH₂)₅COOH |
| cyclohexanecarboxylic acid | | C₆H₁₁COOH |
| benzenecarboxylic acid | benzoic acid | C₆H₅COOH |
| | carboxybenzene | |
| | dracylic acid | |
| 2-hydroxybenzoic acid | salicylic acid | HOC₆H₄COOH |
| octanoic acid | caprylic acid | CH₃(CH₂)6COOH |
| benzene-1,2-dicarboxylic acid | Phthalic acid | C₆H₄(COOH)₂ |
| nonanoic acid | pelargonic acid | CH₃(CH₂)₇COOH |
| | 1-octanecarboxylic acid | |
| benzene-1,3,5-tricarboxylic | trimesic acid | C₆H₃(COOH)₃ |
| acid | | |
| (E)-3-phenylprop-2-enoic acid | cinnamic acid | C₆H₅CH═CHCOOH |
| | trans-cinnamic acid | |
| | phenylacrylic acid | |
| | cinnamylic acid | |
| | 3-phenylacrylic acid | |
| | (E)-cinnamic acid | |
| | benzenepropenoic acid | |
| | isocinnamic acid | |
| decanoic acid | capric acid | CH₃(CH₂)₈COOH |
| decanedioic acid | sebacic acid | HOOC(CH₂)₈COOH |
| | 1,8-octanedicarboxylic acid | |
| undecanoic acid | hendecanoic acid | CH₃(CH₂)₉COOH |
| dodecanoic acid | lauric acid | CH₃(CH₂)₁₀COOH |
| | dodecylic acid | |
| | dodecoic acid | |
| | laurostearic acid | |
| | fulvic acid | |
| | 1-undecanecarboxylic acid | |
| | duodecylic acid | |
| benzene-1,2,3,4,5,6- | mellitic acid | C₆(COOH)₆ |
| hexacarboxylic acid | graphitic acid | |
| | benzenehexacarboxylic acid | |

### Cellulolytic Microbial Abundance in Grass Aerobic Fermentation

In certain embodiments, the cellulosic feedstock comprises grass and the fermentation is aerobic. In certain embodiments, the grass is not switchgrass. Appendix A (which is incorporated herein by reference), is a list of cellulolytic bacterial microorganisms ordered by their abundance as detected when the population of cellulolytic bacterial microorganisms was adapted to grass feedstock fermented under aerobic conditions. Certain embodiments are drawn to the use of one or more of the cellulolytic bacterial microorganisms in Appendix A. In certain embodiments, the most abundant cellulolytic bacterial microorganism and/or most abundant cellulolytic bacterial microorganisms correlate to or approximate the most abundant cellulolytic bacterial microorganisms disclosed in Appendix A. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic bacterial microorganisms in the fermentation solution and/or in the inoculum are selected from the group consisting of the top 100 most abundant types of cellulolytic bacterial microorganisms shown in Appendix A. In certain embodiments, at least 3, 4, 5, 10, 15, 20, 25, 30, 40, 50, 60, 70, or more of the top 75 most abundant types of cellulolytic bacterial microorganisms in the fermentation solution and/or in the inoculum are selected from the group consisting of the top 75 most abundant types of cellulolytic bacterial microorganisms shown in Appendix A. In certain embodiments, at least 2, 3, 4, 5, 10, 15, 20, 25, 30, 40, or more of the top 50 most abundant types of cellulolytic bacterial microorganisms in the fermentation solution and/or in the inoculum are selected from the group consisting of the top 50 most abundant types of cellulolytic bacterial microorganisms shown in Appendix A. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 26A (Tables 26A and 26B are derived from Appendix A). In certain embodiments, the top 25 most abundant types of cellulolytic bacterial microorganisms in the fermentation solution and/or in the inoculum consist of the cellulolytic microorganisms in Table 26A. In certain embodiments, at least 1, 2, 3, 4, 5, 6, 7, 8, 9 or all 10 of the top 10 most abundant types of cellulolytic bacterial microorganisms in the fermentation solution and/or in the inoculum are selected from the group consisting of the cellulolytic bacterial microorganisms in Table 26B. In certain embodiments, at least 1, 2, 3, 4, or all 5 of the top 5 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, (ii) D_0_Bacteria; D_1_Bacteroidetes; D_2_SB-1; Other; Other; Other; Other, (iii) D_0_Bacteria; D_1_Bacteroidetes; D_2_vadinHA17; Other; Other; Other; Other, (iv) D_0_Bacteria; D_1_Firmicutes; D_2_Clostridia; D_3_Clostridiales; D_4_Family XI; D_5_Sedimentibacter; Other, and (v) D_0_Bacteria; D_1_Bacteroidetes; D_2_Bacteroidia; D_3_Bacteroidales; D_4_Rikenellaceae; D_5_vadinBC27 wastewater-sludge group; Other. In certain embodiments, at least 1, 2, or all 3 of the top 3 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, (ii) D_0_Bacteria; D_1_Bacteroidetes; D_2_SB-1; Other; Other; Other; Other, and (iii) D_0_Bacteria; D_1_Bacteroidetes; D_2_vadinHA17; Other; Other; Other; Other. In certain embodiments, at least 1 or both of the top 2 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, and (ii) D_0_Bacteria; D_1_Bacteroidetes; D_2_SB-1; Other; Other; Other; Other. In any of the above, the cellulolytic bacterial microorganisms in the fermentation solution and/or in the inoculum have been adapted to fermentation of grass under aerobic conditions. Further, in certain embodiments, any of the above compositions of cellulolytic bacterial microorganisms can be used for fermentation of grass under anaerobic conditions, fermentation of guar gum under aerobic or anaerobic conditions, or fermentation of a feedstock other than grass, such as but not limited leaves, phragmites, or cattails, under aerobic or anaerobic conditions.

In certain embodiments, the cellulosic feedstock comprises grass and the fermentation is aerobic. In certain embodiments, the grass is not switchgrass. Appendix B (which is incorporated herein by reference), is a list of all cellulolytic microorganisms ordered by their abundance as detected when the population of cellulolytic microorganisms was adapted to grass feedstock fermented under aerobic conditions. Certain embodiments are drawn to the use of one or more of the cellulolytic microorganisms in Appendix B, whether bacterial or non-bacterial. In certain embodiments, the most abundant cellulolytic microorganism and/or most abundant cellulolytic microorganisms correlate to or approximate the most abundant cellulolytic microorganisms disclosed in Appendix B. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic microorganisms in the fermentation solution and/or in the inoculum are selected from the group consisting of the top 100 most abundant types of cellulolytic microorganisms shown in Appendix B. In certain embodiments, at least 3, 4, 5, 10, 15, 20, 25, 30, 40, 50, 60, 70, or more of the top 75 most abundant types of cellulolytic microorganisms in the fermentation solution and/or in the inoculum are selected from the group consisting of the top 75 most abundant types of cellulolytic microorganisms shown in Appendix B. In certain embodiments, at least 2, 3, 4, 5, 10, 15, 20, 25, 30, 40, or more of the top 50 most abundant types of cellulolytic microorganisms in the fermentation solution and/or in the inoculum are selected from the group consisting of the top 50 most abundant types of cellulolytic microorganisms shown in Appendix B. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 27A (Table 27A, Table 27B, and Table 27C are derived from Appendix B). In certain embodiments, the top 25 most abundant types of cellulolytic microorganisms in the fermentation solution and/or in the inoculum consist of the cellulolytic microorganisms in Table 27A. In certain embodiments, at least 1, 2, 3, 4, 5, 6, 7, 8, 9, 10, 11, 12, 13, 14 or all 15 of the top 15 most abundant types of cellulolytic microorganisms in the fermentation solution and/or in the inoculum are selected from the group consisting of the cellulolytic microorganisms in Table 27B. In certain embodiments, at least 1, 2, 3, 4, 5, 6, 7, 8, 9 or all 10 of the top 10 most abundant types of cellulolytic microorganisms in the fermentation solution and/or in the inoculum are selected from the group consisting of the cellulolytic microorganisms in Table 27C. In certain embodiments, at least 1, 2, 3, 4, or all 5 of the top 5 most abundant types of cellulolytic microorganisms in the fermentation solution and/or in the inoculum are selected from the group consisting of: (i) sk_Bacteria; k_Bacteria incertaesedis; p_Proteobacteria; Other; Other; Other; Other; Other, (ii) sk_Bacteria; k_Bacteria incertaesedis; p_Firmicutes; c_Bacilli; o_Bacillales; f_Planococcaceae; g_Sporosarcina; s_Sporosarcina psychrophila, (iii) sk_Archaea; k_Archaea incertaesedis; p_Euryarchaeota; c_Methanomicrobia; o_Methanosarcinales; f_Methanosaetaceae; g_Methanothrix; s_Methanothrix soehngenii, (iv) sk_Eukaryota; k_Metazoa; p_Chordata; c_Mammalia; o_Primates; f_Hominidae; g_Homo; s_Homo sapiens, and (v) sk_Bacteria; k_Bacteria incertaesedis; p_Actinobacteria; c_Actinobacteria; Other; Other; Other; Other. In any of the above, the cellulolytic microorganisms in the fermentation solution and/or in the inoculum have been adapted to fermentation of grass under aerobic conditions. Further, in certain embodiments, any of the above compositions of cellulolytic microorganisms can be used for fermentation of grass under anaerobic conditions, fermentation of guar gum under aerobic or anaerobic conditions, or fermentation of a feedstock other than grass, such as but not limited leaves, phragmites, or cattails, under aerobic or anaerobic conditions.

In certain embodiments, the cellulosic feedstock comprises grass and the fermentation is aerobic. In certain embodiments, the grass is not switchgrass. Appendix B (which is incorporated herein by reference), is a list of all cellulolytic microorganisms ordered by their abundance as detected when the population of cellulolytic microorganisms was adapted to grass feedstock fermented under aerobic conditions. Certain embodiments are drawn to the use of one or more of the cellulolytic non-bacterial microorganisms in Appendix B. In certain embodiments, the most abundant cellulolytic non-bacterial microorganism and/or most abundant cellulolytic non-bacterial microorganisms correlate to or approximate the most abundant cellulolytic non-bacterial microorganisms disclosed in Appendix B. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic non-bacterial microorganisms in the fermentation solution and/or in the inoculum are selected from the group consisting of the top 100 most abundant types of cellulolytic non-bacterial microorganisms shown in Appendix B. In certain embodiments, at least 3, 4, 5, 10, 15, 20, 25, 30, 40, 50, 60, 70, or more of the top 75 most abundant types of cellulolytic non-bacterial microorganisms in the fermentation solution and/or in the inoculum are selected from the group consisting of the top 75 most abundant types of cellulolytic non-bacterial microorganisms shown in Appendix B. In certain embodiments, at least 2, 3, 4, 5, 10, 15, 20, 25, 30, 40, or more of the top 50 most abundant types of cellulolytic non-bacterial microorganisms in the fermentation solution and/or in the inoculum are selected from the group consisting of the top 50 most abundant types of cellulolytic non-bacterial microorganisms shown in Appendix B. In certain embodiments, at least 1, 2, 3, 4, 5, 10, 15, 20, or more of the top 25 most abundant types of cellulolytic non-bacterial microorganisms in the fermentation solution and/or in the inoculum are selected from the group consisting of the top 25 most abundant types of cellulolytic non-bacterial microorganisms shown in Appendix B. In certain embodiments, the top 25 most abundant types of cellulolytic non-bacterial microorganisms in the fermentation solution and/or in the inoculum consist of the top 25 most abundant types of cellulolytic non-bacterial microorganisms shown in Appendix B. In certain embodiments, at least 1, 2, 3, 4, 5, 6, 7, 8, 9, 10, 11, 12, 13, 14 or all 15 of the top 15 most abundant types of cellulolytic non-bacterial microorganisms in the fermentation solution and/or in the inoculum are selected from the group consisting of the top 15 most abundant types of cellulolytic non-bacterial microorganisms shown in Appendix B. In certain embodiments, at least 1, 2, 3, 4, 5, 6, 7, 8, 9 or all 10 of the top 10 most abundant types of cellulolytic non-bacterial microorganisms in the fermentation solution and/or in the inoculum are selected from the group consisting of the top 10 most abundant types of cellulolytic non-bacterial microorganisms shown in Appendix B. In certain embodiments, at least 1, 2, 3, 4, or all 5 of the top 5 most abundant types of cellulolytic non-bacterial microorganisms in the fermentation solution and/or in the inoculum are selected from the top 5 most abundant types of cellulolytic non-bacterial microorganisms shown in Appendix B. In any of the above, the cellulolytic non-bacterial microorganisms in the fermentation solution and/or in the inoculum have been adapted to fermentation of grass under aerobic conditions. Further, in certain embodiments, any of the above compositions of cellulolytic non-bacterial microorganisms can be used for fermentation of grass under anaerobic conditions, fermentation of guar gum under aerobic or anaerobic conditions, or fermentation of a feedstock other than grass, such as but not limited leaves, phragmites, or cattails, under aerobic or anaerobic conditions.

### Cellulolytic Microbial Abundance in Grass Anaerobic Fermentation

In certain embodiments, the cellulosic feedstock comprises grass and the fermentation is anaerobic. In certain embodiments, the grass is not switchgrass. Appendix C (which is incorporated herein by reference), is a list of cellulolytic bacterial microorganisms ordered by their abundance as detected when the population of cellulolytic bacterial microorganisms was adapted to grass feedstock fermented under anaerobic conditions. Certain embodiments are drawn to the use of one or more of the cellulolytic bacterial microorganisms in Appendix C. In certain embodiments, the most abundant cellulolytic bacterial microorganism and/or most abundant cellulolytic bacterial microorganisms correlate to or approximate the most abundant cellulolytic bacterial microorganisms disclosed in Appendix C. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic bacterial microorganisms in the fermentation solution and/or in the inoculum are selected from the group consisting of the top 100 most abundant types of cellulolytic bacterial microorganisms shown in Appendix C. In certain embodiments, at least 3, 4, 5, 10, 15, 20, 25, 30, 40, 50, 60, 70, or more of the top 75 most abundant types of cellulolytic bacterial microorganisms in the fermentation solution and/or in the inoculum are selected from the group consisting of the top 75 most abundant types of cellulolytic bacterial microorganisms shown in Appendix C. In certain embodiments, at least 2, 3, 4, 5, 10, 15, 20, 25, 30, 40, or more of the top 50 most abundant types of cellulolytic bacterial microorganisms in the fermentation solution and/or in the inoculum are selected from the group consisting of the top 50 most abundant types of cellulolytic bacterial microorganisms shown in Appendix C. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 28A (Table 28A and Table 28B are derived from Appendix C). In certain embodiments, the top 25 most abundant types of cellulolytic bacterial microorganisms in the fermentation solution and/or in the inoculum consist of the cellulolytic microorganisms in Table 28A. In certain embodiments, at least 1, 2, 3, 4, 5, 6, 7, 8, 9 or all 10 of the top 10 most abundant types of cellulolytic bacterial microorganisms in the fermentation solution and/or in the inoculum are selected from the group consisting of the cellulolytic bacterial microorganisms in Table 28B. In certain embodiments, at least 1, 2, 3, 4, or all 5 of the top 5 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, (ii) D_0_Bacteria; D_1_Bacteroidetes; D_2_SB-1; Other; Other; Other; Other, (iii) D_0_Bacteria; D_1_Bacteroidetes; D_2_Bacteroidia; D_3_Bacteroidales; D_4_Rikenellaceae; D_5_vadinBC27 wastewater-sludge group; Other, (iv) D_0_Bacteria; D_1_Bacteroidetes; D_2_vadinHA17; Other; Other; Other; Other, and (v) D_0_Bacteria; D_1 Synergistetes; D_2 Synergistia; D_3 Synergistales; D_4 Synergistaceae; D_5 Thermovirga; Other. In certain embodiments, at least 1, 2, or all 3 of the top 3 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, (ii) D_0_Bacteria; D_1_Bacteroidetes; D_2_SB-1; Other; Other; Other; Other, and (iii) D_0_Bacteria; D_1_Bacteroidetes; D_2_Bacteroidia; D_3_Bacteroidales; D_4_Rikenellaceae; D_5_vadinBC27 wastewater-sludge group; Other. In certain embodiments, at least 1 or both of the top 2 most abundant types of cellulolytic bacterial microorganisms in the fermentation solution and/or in the inoculum are selected from the group consisting of: (i) D_0_Bacteria; D_1_Chloroflexi; D_2_Anaerolineae; D_3_Anaerolineales; D_4_Anaerolineaceae; Other; Other, and (ii) D_0_Bacteria; D_1_Bacteroidetes; D_2_SB-1; Other; Other; Other; Other. In any of the above, the cellulolytic bacterial microorganisms in the fermentation solution and/or in the inoculum have been adapted to fermentation of grass under anaerobic conditions. Further, in certain embodiments, any of the above compositions of cellulolytic bacterial microorganisms can be used for fermentation of grass under aerobic conditions, fermentation of guar gum under aerobic or anaerobic conditions, or fermentation of a feedstock other than grass, such as but not limited leaves, phragmites, or cattails, under aerobic or anaerobic conditions.

In certain embodiments, the cellulosic feedstock comprises grass and the fermentation is anaerobic. In certain embodiments, the grass is not switchgrass. Appendix D (which is incorporated herein by reference), is a list of all cellulolytic microorganisms ordered by their abundance as detected when the population of cellulolytic microorganisms was adapted to grass feedstock fermented under anaerobic conditions. Certain embodiments are drawn to the use of one or more of the cellulolytic microorganisms in Appendix D, whether bacterial or non-bacterial. In certain embodiments, the most abundant cellulolytic microorganism and/or most abundant cellulolytic microorganisms correlate to or approximate the most abundant cellulolytic microorganisms disclosed in Appendix D. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic microorganisms in the fermentation solution and/or in the inoculum are selected from the group consisting of the top 100 most abundant types of cellulolytic microorganisms shown in Appendix D. In certain embodiments, at least 3, 4, 5, 10, 15, 20, 25, 30, 40, 50, 60, 70, or more of the top 75 most abundant types of cellulolytic microorganisms in the fermentation solution and/or in the inoculum are selected from the group consisting of the top 75 most abundant types of cellulolytic microorganisms shown in Appendix D. In certain embodiments, at least 2, 3, 4, 5, 10, 15, 20, 25, 30, 40, or more of the top 50 most abundant types of cellulolytic microorganisms in the fermentation solution and/or in the inoculum are selected from the group consisting of the top 50 most abundant types of cellulolytic microorganisms shown in Appendix D. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 29A (Table 29A, Table 29B, and Table 29C are derived from Appendix D). In certain embodiment, the top 25 most abundant types of cellulolytic microorganisms in the fermentation solution and/or in the inoculum consist of the cellulolytic microorganisms in Table 29A. In certain embodiments, at least 1, 2, 3, 4, 5, 6, 7, 8, 9, 10, 11, 12, 13, 14 or all 15 of the top 15 most abundant types of cellulolytic microorganisms in the fermentation solution and/or in the inoculum are selected from the group consisting of the cellulolytic microorganisms in Table 29B. In certain embodiments, at least 1, 2, 3, 4, 5, 6, 7, 8, 9 or all 10 of the top 10 most abundant types of cellulolytic microorganisms in the fermentation solution and/or in the inoculum are selected from the group consisting of the cellulolytic microorganisms in Table 29C. In certain embodiments, at least 1, 2, 3, 4, or all 5 of the top 5 most abundant types of cellulolytic microorganisms in the fermentation solution and/or in the inoculum are selected from the group consisting of: (i) sk_Archaea; k_Archaea incertaesedis; p_Euryarchaeota; c_Methanomicrobia; o_Methanosarcinales; f_Methanosaetaceae; g_Methanothrix; s_Methanothrix soehngenii, (ii) sk_Bacteria; k_Bacteria incertaesedis; p_Actinobacteria; c_Actinobacteria; Other; Other; Other; Other, (iii) sk_Bacteria; k_Bacteria incertae sedis; p_Proteobacteria; Other; Other; Other; Other; Other, (iv) sk_Bacteria; k_Bacteria incertae sedis; p_Candidatus Cloacimonetes; c_Candidatus Cloacimonetes incertae sedis; o_Candidatus Cloacimonetes incertae sedis; f_Candidatus Cloacimonetes incertae sedis; g_Candidatus Cloacimonas; s_Candidatus Cloacimonas acidaminovorans, and (v) sk_Bacteria; k_Bacteria incertaesedis; p_Actinobacteria; c_Actinobacteria; o_Streptomycetales; f_Streptomycetaceae; g_Streptomyces; Other. In any of the above, the cellulolytic microorganisms in the fermentation solution and/or in the inoculum have been adapted to fermentation of grass under anaerobic conditions. Further, in certain embodiments, any of the above compositions of cellulolytic microorganisms can be used for fermentation of grass under aerobic conditions, fermentation of guar gum under aerobic or anaerobic conditions, or fermentation of a feedstock other than grass, such as but not limited leaves, phragmites, or cattails, under aerobic or anaerobic conditions.

In certain embodiments, the cellulosic feedstock comprises grass and the fermentation is anaerobic. In certain embodiments, the grass is not switchgrass. Appendix D (which is incorporated herein by reference), is a list of all cellulolytic microorganisms ordered by their abundance as detected when the population of cellulolytic microorganisms was adapted to grass feedstock fermented under anaerobic conditions. Certain embodiments are drawn to the use of one or more of the cellulolytic non-bacterial microorganisms in Appendix D. In certain embodiments, the most abundant cellulolytic non-bacterial microorganism and/or most abundant cellulolytic non-bacterial microorganisms correlate to or approximate the most abundant cellulolytic non-bacterial microorganisms disclosed in Appendix D. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic non-bacterial microorganisms in the fermentation solution and/or in the inoculum are selected from the group consisting of the top 100 most abundant types of cellulolytic non-bacterial microorganisms shown in Appendix D. In certain embodiments, at least 3, 4, 5, 10, 15, 20, 25, 30, 40, 50, 60, 70, or more of the top 75 most abundant types of cellulolytic non-bacterial microorganisms in the fermentation solution and/or in the inoculum are selected from the group consisting of the top 75 most abundant types of cellulolytic non-bacterial microorganisms shown in Appendix D. In certain embodiments, at least 2, 3, 4, 5, 10, 15, 20, 25, 30, 40, or more of the top 50 most abundant types of cellulolytic non-bacterial microorganisms in the fermentation solution and/or in the inoculum are selected from the group consisting of the top 50 most abundant types of cellulolytic non-bacterial microorganisms shown in Appendix D. In certain embodiments, at least 1, 2, 3, 4, 5, 10, 15, 20, or more of the top 25 most abundant types of cellulolytic non-bacterial microorganisms in the fermentation solution and/or in the inoculum are selected from the group consisting of the top 25 most abundant types of cellulolytic non-bacterial microorganisms shown in Appendix D. In certain embodiment, the top 25 most abundant types of cellulolytic non-bacterial microorganisms in the fermentation solution and/or in the inoculum consist of the top 25 most abundant types of cellulolytic non-bacterial microorganisms shown in Appendix D. In certain embodiments, at least 1, 2, 3, 4, 5, 6, 7, 8, 9, 10, 11, 12, 13, 14 or all 15 of the top 15 most abundant types of cellulolytic non-bacterial microorganisms in the fermentation solution and/or in the inoculum are selected from the group consisting of the top 15 most abundant types of cellulolytic non-bacterial microorganisms shown in Appendix D. In certain embodiments, at least 1, 2, 3, 4, 5, 6, 7, 8, 9 or all 10 of the top 10 most abundant types of cellulolytic non-bacterial microorganisms in the fermentation solution and/or in the inoculum are selected from the group consisting of the top 10 most abundant types of cellulolytic non-bacterial microorganisms shown in Appendix D. In certain embodiments, at least 1, 2, 3, 4, or all 5 of the top 5 most abundant types of cellulolytic non-bacterial microorganisms in the fermentation solution and/or in the inoculum are selected from the group consisting of the top 5 most abundant types of cellulolytic non-bacterial microorganisms shown in Appendix D. In any of the above, the cellulolytic non-bacterial microorganisms in the fermentation solution and/or in the inoculum have been adapted to fermentation of grass under anaerobic conditions. Further, in certain embodiments, any of the above compositions of cellulolytic non-bacterial microorganisms can be used for fermentation of grass under aerobic conditions, fermentation of guar gum under aerobic or anaerobic conditions, or fermentation of a feedstock other than grass, such as but not limited leaves, phragmites, or cattails, under aerobic or anaerobic conditions.

### Cellulolytic Microbial Abundance in Guar Gum Aerobic Fermentation

In certain embodiments, the cellulosic feedstock comprises guar gum and the fermentation is aerobic. Appendix E (which is incorporated herein by reference), is a list of cellulolytic bacterial microorganisms ordered by their abundance as detected when the population of cellulolytic bacterial microorganisms was adapted to guar gum feedstock fermented under aerobic conditions. Certain embodiments are drawn to the use of one or more of the cellulolytic bacterial microorganisms in Appendix E. In certain embodiments, the most abundant cellulolytic bacterial microorganism and/or most abundant cellulolytic bacterial microorganisms correlate to or approximate the most abundant cellulolytic bacterial microorganisms disclosed in Appendix E. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic bacterial microorganisms in the fermentation solution and/or in the inoculum are selected from the group consisting of the top 100 most abundant types of cellulolytic bacterial microorganisms shown in Appendix E. In certain embodiments, at least 3, 4, 5, 10, 15, 20, 25, 30, 40, 50, 60, 70, or more of the top 75 most abundant types of cellulolytic bacterial microorganisms in the fermentation solution and/or in the inoculum are selected from the group consisting of the top 75 most abundant types of cellulolytic bacterial microorganisms shown in Appendix E. In certain embodiments, at least 2, 3, 4, 5, 10, 15, 20, 25, 30, 40, or more of the top 50 most abundant types of cellulolytic bacterial microorganisms in the fermentation solution and/or in the inoculum are selected from the group consisting of the top 50 most abundant types of cellulolytic bacterial microorganisms shown in Appendix E. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 30A (Table 30A and Table 30B are derived from Appendix E). In certain embodiments, the top 25 most abundant types of cellulolytic bacterial microorganisms in the fermentation solution and/or in the inoculum consist of the cellulolytic microorganisms in Table 30A. In certain embodiments, at least 1, 2, 3, 4, 5, 6, 7, 8, 9 or all 10 of the top 10 most abundant types of cellulolytic bacterial microorganisms in the fermentation solution and/or in the inoculum are selected from the group consisting of the cellulolytic bacterial microorganisms in Table 30B. In certain embodiments, at least 1, 2, 3, 4, or all 5 of the top 5 most abundant types of cellulolytic bacterial microorganisms in the fermentation solution and/or in the inoculum are selected from the group consisting of: (i) D_0_Bacteria; D_1_Bacteroidetes; D_2_vadinHA17; Other; Other; Other; Other, (ii) D_0_Bacteria; D_1_Chloroflexi; D_2_Anaerolineae; D_3_Anaerolineales; D_4_Anaerolineaceae; Other; Other, (iii) D_0_Bacteria; D_1_Bacteroidetes; D_2_Sphingobacteriia; D_3_Sphingobacteriales; D_4_W CHB1-69; Other; Other, (iv) D_0_Bacteria; D_1_Bacteroidetes; D_2_SB-1; Other; Other; Other; Other, and (v) D_0_Bacteria; D_1_Chloroflexi; Other; Other; Other; Other; Other. In certain embodiments, at least 1, 2, or all 3 of the top 3 most abundant types of cellulolytic bacterial microorganisms in the fermentation solution and/or in the inoculum are selected from the group consisting of: (i) D_0_Bacteria; D_1_Bacteroidetes; D_2_vadinHA17; Other; Other; Other; Other, (ii) D_0_Bacteria; D_1_Chloroflexi; D_2_Anaerolineae; D_3_Anaerolineales; D_4_Anaerolineaceae; Other; Other, and (iii) D_0_Bacteria; D_1_Bacteroidetes; D_2_Sphingobacteriia; D_3_Sphingobacteriales; D_4_W CHB1-69; Other; Other. In certain embodiments, at least 1 or both of the top 2 most abundant types of cellulolytic bacterial microorganisms in the fermentation solution and/or in the inoculum are selected from the group consisting of: (i) D_0_Bacteria; D_1_Bacteroidetes; D_2_vadinHA17; Other; Other; Other; Other, and (ii) D_0_Bacteria; D_1_Chloroflexi; D_2_Anaerolineae; D_3_Anaerolineales; D_4_Anaerolineaceae; Other; Other. In any of the above, the cellulolytic bacterial microorganisms in the fermentation solution and/or in the inoculum have been adapted to fermentation of guar gum under aerobic conditions. Further, in certain embodiments, any of the above compositions of cellulolytic bacterial microorganisms can be used for fermentation of guar gum under anaerobic conditions, fermentation of grass under aerobic or anaerobic conditions, or fermentation of a feedstock other than guar gum, such as but not limited leaves, phragmites, or cattails, under aerobic or anaerobic conditions.

In certain embodiments, the cellulosic feedstock comprises guar gum and the fermentation is aerobic. Appendix F (which is incorporated herein by reference), is a list of all cellulolytic microorganisms ordered by their abundance as detected when the population of cellulolytic microorganisms was adapted to guar gum feedstock fermented under aerobic conditions. Certain embodiments are drawn to the use of one or more of the cellulolytic microorganisms in Appendix F, whether bacterial or non-bacterial. In certain embodiments, the most abundant cellulolytic microorganism and/or most abundant cellulolytic microorganisms correlate to or approximate the most abundant cellulolytic microorganisms disclosed in Appendix F. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic microorganisms in the fermentation solution and/or in the inoculum are selected from the group consisting of the top 100 most abundant types of cellulolytic microorganisms shown in Appendix F. In certain embodiments, at least 3, 4, 5, 10, 15, 20, 25, 30, 40, 50, 60, 70, or more of the top 75 most abundant types of cellulolytic microorganisms in the fermentation solution and/or in the inoculum are selected from the group consisting of the top 75 most abundant types of cellulolytic microorganisms shown in Appendix F. In certain embodiments, at least 2, 3, 4, 5, 10, 15, 20, 25, 30, 40, or more of the top 50 most abundant types of cellulolytic microorganisms in the fermentation solution and/or in the inoculum are selected from the group consisting of the top 50 most abundant types of cellulolytic microorganisms shown in Appendix F. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 31A (Table 31A, Table 31B, and Table 31C are derived from Appendix F). In certain embodiment, the top 25 most abundant types of cellulolytic microorganisms in the fermentation solution and/or in the inoculum consist of the cellulolytic microorganisms in Table 31A. In certain embodiments, at least 1, 2, 3, 4, 5, 6, 7, 8, 9, 10, 11, 12, 13, 14 or all 15 of the top 15 most abundant types of cellulolytic microorganisms in the fermentation solution and/or in the inoculum are selected from the group consisting of the cellulolytic microorganisms in Table 31B. In certain embodiments, at least 1, 2, 3, 4, 5, 6, 7, 8, 9 or all 10 of the top 10 most abundant types of cellulolytic microorganisms in the fermentation solution and/or in the inoculum are selected from the group consisting of the cellulolytic microorganisms in Table 31C. In certain embodiments, at least 1, 2, 3, 4, or all 5 of the top 5 most abundant types of cellulolytic microorganisms in the fermentation solution and/or in the inoculum are selected from the group consisting of: (i) sk_Bacteria; k_Bacteria incertaesedis; p_Proteobacteria; Other; Other; Other; Other; Other, (ii) sk_Bacteria; k_Bacteria incertaesedis; p_Actinobacteria; c_Actinobacteria; Other; Other; Other; Other, (iii) sk_Bacteria; k_Bacteria incertaesedis; p_Proteobacteria; c_Betaproteobacteria; o_Burkholderiales; f_Burkholderiaceae; g_Burkholderia; Other, (iv) sk_Bacteria; k_Bacteria incertaesedis; p_Actinobacteria; c_Actinobacteria; o_Streptomycetales; f_Streptomycetaceae; g_Streptomyces; Other, and (v) sk_Bacteria; k_Bacteria incertaesedis; p_Proteobacteria; c_Gammaproteobacteria; o_Pseudomonadales; f_Pseudomonadaceae; g_Pseudomonas; Other. In any of the above, the cellulolytic microorganisms in the fermentation solution and/or in the inoculum have been adapted to fermentation of guar gum under aerobic conditions. Further, in certain embodiments, any of the above compositions of cellulolytic microorganisms can be used for fermentation of guar gum under anaerobic conditions, fermentation of grass under aerobic or anaerobic conditions, or fermentation of a feedstock other than guar gum, such as but not limited leaves, phragmites, or cattails, under aerobic or anaerobic conditions.

In certain embodiments, the cellulosic feedstock comprises guar gum and the fermentation is aerobic. Appendix F (which is incorporated herein by reference), is a list of all cellulolytic microorganisms ordered by their abundance as detected when the population of cellulolytic microorganisms was adapted to guar gum feedstock fermented under aerobic conditions. Certain embodiments are drawn to the use of one or more of the cellulolytic non-bacterial microorganisms in Appendix F. In certain embodiments, the most abundant cellulolytic non-bacterial microorganism and/or most abundant cellulolytic non-bacterial microorganisms correlate to or approximate the most abundant cellulolytic non-bacterial microorganisms disclosed in Appendix F. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic non-bacterial microorganisms in the fermentation solution and/or in the inoculum are selected from the group consisting of the top 100 most abundant types of cellulolytic non-bacterial microorganisms shown in Appendix F. In certain embodiments, at least 3, 4, 5, 10, 15, 20, 25, 30, 40, 50, 60, 70, or more of the top 75 most abundant types of cellulolytic non-bacterial microorganisms in the fermentation solution and/or in the inoculum are selected from the group consisting of the top 75 most abundant types of cellulolytic non-bacterial microorganisms shown in Appendix F. In certain embodiments, at least 2, 3, 4, 5, 10, 15, 20, 25, 30, 40, or more of the top 50 most abundant types of cellulolytic non-bacterial microorganisms in the fermentation solution and/or in the inoculum are selected from the group consisting of the top 50 most abundant types of cellulolytic non-bacterial microorganisms shown in Appendix F. In certain embodiments, at least 1, 2, 3, 4, 5, 10, 15, 20, or more of the top 25 most abundant types of cellulolytic non-bacterial microorganisms in the fermentation solution and/or in the inoculum are selected from the group consisting of the top 25 most abundant types of cellulolytic non-bacterial microorganisms shown in Appendix F. In certain embodiment, the top 25 most abundant types of cellulolytic non-bacterial microorganisms in the fermentation solution and/or in the inoculum consist of the top 25 most abundant types of cellulolytic non-bacterial microorganisms shown in Appendix F. In certain embodiments, at least 1, 2, 3, 4, 5, 6, 7, 8, 9, 10, 11, 12, 13, 14 or all 15 of the top 15 most abundant types of cellulolytic non-bacterial microorganisms in the fermentation solution and/or in the inoculum are selected from the group consisting of the top 15 most abundant types of cellulolytic non-bacterial microorganisms shown in Appendix F. In certain embodiments, at least 1, 2, 3, 4, 5, 6, 7, 8, 9 or all 10 of the top 10 most abundant types of cellulolytic non-bacterial microorganisms in the fermentation solution and/or in the inoculum are selected from the group consisting of the top 10 most abundant types of cellulolytic non-bacterial microorganisms shown in Appendix F. In certain embodiments, at least 1, 2, 3, 4, or all 5 of the top 5 most abundant types of cellulolytic non-bacterial microorganisms in the fermentation solution and/or in the inoculum are selected from the group consisting of the top 5 most abundant types of cellulolytic non-bacterial microorganisms shown in Appendix F. In any of the above, the cellulolytic non-bacterial microorganisms in the fermentation solution and/or in the inoculum have been adapted to fermentation of guar gum under aerobic conditions. Further, in certain embodiments, any of the above compositions of cellulolytic non-bacterial microorganisms can be used for fermentation of guar gum under anaerobic conditions, fermentation of grass under aerobic or anaerobic conditions, or fermentation of a feedstock other than guar gum, such as but not limited leaves, phragmites, or cattails, under aerobic or anaerobic conditions.

### Cellulolytic Microbial Abundance in Guar Gum Anaerobic Fermentation

In certain embodiments, the cellulosic feedstock comprises guar gum and the fermentation is anaerobic. Appendix G (which is incorporated herein by reference), is a list of cellulolytic bacterial microorganisms ordered by their abundance as detected when the population of cellulolytic bacterial microorganisms was adapted to guar gum feedstock fermented under anaerobic conditions. Certain embodiments are drawn to the use of one or more of the cellulolytic bacterial microorganisms in Appendix G. In certain embodiments, the most abundant cellulolytic bacterial microorganism and/or most abundant cellulolytic bacterial microorganisms correlate to or approximate the most abundant cellulolytic bacterial microorganisms disclosed in Appendix G. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic bacterial microorganisms in the fermentation solution and/or in the inoculum are selected from the group consisting of the top 100 most abundant types of cellulolytic bacterial microorganisms shown in Appendix G. In certain embodiments, at least 3, 4, 5, 10, 15, 20, 25, 30, 40, 50, 60, 70, or more of the top 75 most abundant types of cellulolytic bacterial microorganisms in the fermentation solution and/or in the inoculum are selected from the group consisting of the top 75 most abundant types of cellulolytic bacterial microorganisms shown in Appendix G. In certain embodiments, at least 2, 3, 4, 5, 10, 15, 20, 25, 30, 40, or more of the top 50 most abundant types of cellulolytic bacterial microorganisms in the fermentation solution and/or in the inoculum are selected from the group consisting of the top 50 most abundant types of cellulolytic bacterial microorganisms shown in Appendix G. In certain embodiments, at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 32A (Table 32A and Table 32B are derived from Appendix G). In certain embodiments, the top 25 most abundant types of cellulolytic bacterial microorganisms in the fermentation solution and/or in the inoculum consist of the cellulolytic microorganisms in Table 32A. In certain embodiments, at least 1, 2, 3, 4, 5, 6, 7, 8, 9 or all 10 of the top 10 most abundant types of cellulolytic bacterial microorganisms in the fermentation solution and/or in the inoculum are selected from the group consisting of the cellulolytic bacterial microorganisms in Table 32B. In certain embodiments, at least 1, 2, 3, 4, or all 5 of the top 5 most abundant types of cellulolytic bacterial microorganisms in the fermentation solution and/or in the inoculum are selected from the group consisting of: (i) D_0_Bacteria; D_1_Bacteroidetes; D_2_Bacteroidia; D_3_Bacteroidales; D_4 Bacteroidaceae; D_5 Bacteroides; Other 4C_PE Reactor, (ii) D_0_Bacteria; D_1_Bacteroidetes; D_2_vadinHA17; Other; Other; Other; Other, (iii) D_0_Bacteria; D_1_Chloroflexi; D_2_Anaerolineae; D_3_Anaerolineales; D_4_Anaerolineaceae; Other; Other, (iv) D_0_Bacteria; D_1_Proteobacteria; D_2_Epsilonproteobacteria; D_3_Campylobacterales; D_4_Helicobacteraceae; D_5_Sulfurovum; Other, and (v) D_0_Bacteria; D_1_Bacteroidetes; D_2_Sphingobacteriia; D_3_Sphingobacteriales; D_4_W CHB1-69; Other; Other. In certain embodiments, at least 1, 2, or all 3 of the top 3 most abundant types of cellulolytic bacterial microorganisms in the fermentation solution and/or in the inoculum are selected from the group consisting of: (i) D_0_Bacteria; D_1_Bacteroidetes; D_2_Bacteroidia; D_3_Bacteroidales; D_4 Bacteroidaceae; D_5 Bacteroides; Other 4C_PE Reactor, (ii) D_0_Bacteria; D_1_Bacteroidetes; D_2_vadinHA17; Other; Other; Other; Other, and (iii) D_0_Bacteria; D_1_Chloroflexi; D_2_Anaerolineae; D_3_Anaerolineales; D_4_Anaerolineaceae; Other; Other. In certain embodiments, at least 1 or both of the top 2 most abundant types of cellulolytic bacterial microorganisms in the fermentation solution and/or in the inoculum are selected from the group consisting of: (i) D_0_Bacteria; D_1_Bacteroidetes; D_2_Bacteroidia; D_3_Bacteroidales; D_4 Bacteroidaceae; D_5 Bacteroides; Other 4C_PE Reactor, and (ii) D_0_Bacteria; D_1_Bacteroidetes; D_2_vadinHA17; Other; Other; Other; Other. In any of the above, the cellulolytic bacterial microorganisms in the fermentation solution and/or in the inoculum have been adapted to fermentation of guar gum under anaerobic conditions. Further, in certain embodiments, any of the above compositions of cellulolytic bacterial microorganisms can be used for fermentation of guar gum under aerobic conditions, fermentation of grass under aerobic or anaerobic conditions, or fermentation of a feedstock other than guar gum, such as but not limited leaves, phragmites, or cattails, under aerobic or anaerobic conditions.

In certain embodiments, the cellulosic feedstock comprises guar gum and the fermentation is anaerobic. Appendix H (which is incorporated herein by reference), is a list of all cellulolytic microorganisms ordered by their abundance as detected when the population of cellulolytic microorganisms was adapted to guar gum feedstock fermented under anaerobic conditions. Certain embodiments are drawn to the use of one or more of the cellulolytic microorganisms in Appendix H, whether bacterial or non-bacterial. In certain embodiments, the most abundant cellulolytic microorganism and/or most abundant cellulolytic microorganisms correlate to or approximate the most abundant cellulolytic microorganisms disclosed in Appendix H. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic microorganisms in the fermentation solution and/or in the inoculum are selected from the group consisting of the top 100 most abundant types of cellulolytic microorganisms shown in Appendix H. In certain embodiments, at least 3, 4, 5, 10, 15, 20, 25, 30, 40, 50, 60, 70, or more of the top 75 most abundant types of cellulolytic microorganisms in the fermentation solution and/or in the inoculum are selected from the group consisting of the top 75 most abundant types of cellulolytic microorganisms shown in Appendix H. In certain embodiments, at least 2, 3, 4, 5, 10, 15, 20, 25, 30, 40, or more of the top 50 most abundant types of cellulolytic microorganisms in the fermentation solution and/or in the inoculum are selected from the group consisting of the top 50 most abundant types of cellulolytic microorganisms shown in Appendix H. In certain embodiments, at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 33A (Table 33A, Table 33B, and Table 33C are derived from Appendix H). In certain embodiment, the top 25 most abundant types of cellulolytic microorganisms in the fermentation solution and/or in the inoculum consist of the cellulolytic microorganisms in Table 33A. In certain embodiments, at least 1, 2, 3, 4, 5, 6, 7, 8, 9, 10, 11, 12, 13, 14 or all 15 of the top 15 most abundant types of cellulolytic microorganisms in the fermentation solution and/or in the inoculum are selected from the group consisting of the cellulolytic microorganisms in Table 33B. In certain embodiments, at least 1, 2, 3, 4, 5, 6, 7, 8, 9 or all 10 of the top 10 most abundant types of cellulolytic microorganisms in the fermentation solution and/or in the inoculum are selected from the group consisting of the cellulolytic microorganisms in Table 33C. In certain embodiments, at least 1, 2, 3, 4, or all 5 of the top 5 most abundant types of cellulolytic microorganisms in the fermentation solution and/or in the inoculum are selected from the group consisting of: (i) sk_Bacteria; k_Bacteria incertae sedis; p_Proteobacteria; Other; Other; Other; Other; Other, (ii) sk_Bacteria; k_Bacteria incertaesedis; p_Actinobacteria; c_Actinobacteria; Other; Other; Other; Other, (iii) sk_Bacteria; k_Bacteria incertaesedis; p_Proteobacteria; c_Betaproteobacteria; o_Burkholderiales; f_Burkholderiaceae; g_Burkholderia; Other, (iv) sk_Archaea; k_Archaea incertaesedis; p_Euryarchaeota; c_Methanomicrobia; o_Methanosarcinales; f_Methanosaetaceae; g_Methanothrix; s_Methanothrix soehngenii, and (v) sk_Bacteria; k_Bacteria incertaesedis; p_Actinobacteria; c_Actinobacteria; o_Streptomycetales; f_Streptomycetaceae; g_Streptomyces; Other. In any of the above, the cellulolytic microorganisms in the fermentation solution and/or in the inoculum have been adapted to fermentation of guar gum under anaerobic conditions. Further, in certain embodiments, any of the above compositions of cellulolytic microorganisms can be used for fermentation of guar gum under aerobic conditions, fermentation of grass under aerobic or anaerobic conditions, or fermentation of a feedstock other than guar gum, such as but not limited leaves, phragmites, or cattails, under aerobic or anaerobic conditions.

In certain embodiments, the cellulosic feedstock comprises guar gum and the fermentation is anaerobic. Appendix H (which is incorporated herein by reference), is a list of all cellulolytic microorganisms ordered by their abundance as detected when the population of cellulolytic microorganisms was adapted to guar gum feedstock fermented under anaerobic conditions. Certain embodiments are drawn to the use of one or more of the cellulolytic no-bacterial microorganisms in Appendix H. In certain embodiments, the most abundant cellulolytic non-bacterial microorganism and/or most abundant cellulolytic non-bacterial microorganisms correlate to or approximate the most abundant cellulolytic non-bacterial microorganisms disclosed in Appendix H. For example, while it would be prohibitive to list every potential combination which are understood to be disclosed herein, in certain embodiments, at least 5, 10, 15, 20, 25, 30, 40, 50, 60, 70, 80, 90, or more of the top 100 most abundant types of cellulolytic non-bacterial microorganisms in the fermentation solution and/or in the inoculum are selected from the group consisting of the top 100 most abundant types of cellulolytic non-bacterial microorganisms shown in Appendix H. In certain embodiments, at least 3, 4, 5, 10, 15, 20, 25, 30, 40, 50, 60, 70, or more of the top 75 most abundant types of cellulolytic non-bacterial microorganisms in the fermentation solution and/or in the inoculum are selected from the group consisting of the top 75 most abundant types of cellulolytic non-bacterial microorganisms shown in Appendix H. In certain embodiments, at least 2, 3, 4, 5, 10, 15, 20, 25, 30, 40, or more of the top 50 most abundant types of cellulolytic non-bacterial microorganisms in the fermentation solution and/or in the inoculum are selected from the group consisting of the top 50 most abundant types of cellulolytic non-bacterial microorganisms shown in Appendix H. In certain embodiments, at least 1, 2, 3, 4, 5, 10, 15, 20, or more of the top 25 most abundant types of cellulolytic non-bacterial microorganisms in the fermentation solution and/or in the inoculum are selected from the group consisting of the top 25 most abundant types of cellulolytic non-bacterial microorganisms shown in Appendix H. In certain embodiment, the top 25 most abundant types of cellulolytic non-bacterial microorganisms in the fermentation solution and/or in the inoculum consist of the top 25 most abundant types of cellulolytic non-bacterial microorganisms shown in Appendix H. In certain embodiments, at least 1, 2, 3, 4, 5, 6, 7, 8, 9, 10, 11, 12, 13, 14 or all 15 of the top 15 most abundant types of cellulolytic non-bacterial microorganisms in the fermentation solution and/or in the inoculum are selected from the group consisting of the top 15 most abundant types of cellulolytic non-bacterial microorganisms shown in Appendix H. In certain embodiments, at least 1, 2, 3, 4, 5, 6, 7, 8, 9 or all 10 of the top 10 most abundant types of cellulolytic non-bacterial microorganisms in the fermentation solution and/or in the inoculum are selected from the group consisting of the top 10 most abundant types of cellulolytic non-bacterial microorganisms shown in Appendix H. In certain embodiments, at least 1, 2, 3, 4, or all 5 of the top 5 most abundant types of cellulolytic non-bacterial microorganisms in the fermentation solution and/or in the inoculum are selected from the group consisting of the top 5 most abundant types of cellulolytic non-bacterial microorganisms shown in Appendix H. In any of the above, the cellulolytic non-bacterial microorganisms in the fermentation solution and/or in the inoculum have been adapted to fermentation of guar gum under anaerobic conditions. Further, in certain embodiments, any of the above compositions of cellulolytic non-bacterial microorganisms can be used for fermentation of guar gum under aerobic conditions, fermentation of grass under aerobic or anaerobic conditions, or fermentation of a feedstock other than guar gum, such as but not limited leaves, phragmites, or cattails, under aerobic or anaerobic conditions.

In certain embodiments, the cellulolytic microorganisms in the inoculum are adapted or derived from a seed source reservoir. In certain such embodiments, the seed source reservoir is a digester draw from a municipal sewage treatment operation. In certain embodiments, a method disclosed herein comprises deriving cellulolytic microorganisms from a seed source reservoir for use in the fermentation of the cellulosic feedstock, for example, as shown in the Examples. In certain embodiments, during such derivation, the composition of the cellulolytic microorganisms is adapted to the type of cellulosic feedstock and fermentation conditions to be used.

In certain embodiments, derivation phase comprises fermentation of the cellulolytic microorganisms from a seed source reservoir for at least 1 day, 2 days, 3 days, 4 days, 5 days, 6 day, 7 days, at least 8 days, at least 10 days, at least 14 days, at least 21 days, at least 28 days, or at least 30 days, during which time the fermentation reaction can be “re-fed” one, twice, or three times per week by removing a portion of the reaction and replacing with feedstock (e.g., fresh feedstock), nutrient solution, and/or digester sludge. In certain embodiments, about 1%, 2%, 3%, 4%, or 5%, e.g., 3%-5% of the volume is removed. It is understood that more can be removed, e.g., more than 5%, 5% to 10%, or more than 10% can be removed but doing so will delay the amount of time needed to the population of cellulolytic microorganisms to regain their mass. In certain embodiments, the method further comprising cultivating and maintaining the derived cellulolytic microorganisms' composition for use as the inoculum.

In certain embodiments disclosed anywhere herein, the cellulosic feedstock is fermented for about 1, 2, 3, 4, 5, 6, 7, or more days. In certain embodiments, after 1, 2, 3, 4, 5, 6, or 7 days, a portion of the fermentation solution is removed and replaced with a solution comprising unfermented cellulosic feedstock. In certain embodiments, additional inoculum and/or nutrient solution is also added. In certain embodiments, the amount of fermentation solution removed and replaced is about 50%, 60%, 70%, 75%, 80%, 85%, 90%, or 95% of its volume. During the phase of generating VFAs and/or biogas, in a continuously operated batch system, any volume removal up to 80% of total solid and liquid volume or almost all liquid volume removal (i.e., leaving 20% of the total volume in the form of solids) is permissible, so long as there is sufficient active mass of solids left behind in the reactor that contains cellulolytic organisms to ferment a new incoming unfermented feedstock. In certain embodiments, the removal and replacement of a portion of the fermentation solution is repeated at least once after 1, 2, 3, 4, 5, 6, or 7 days. For purposes of determining the fermentation time in a continuously operated batch system, the time is considered reset to day 0 after each volume removal and replacement. In certain embodiments, the cellulosic feedstock is fermented from about 1 to 4 days to produce VFAs. In certain embodiments, the cellulosic feedstock is fermented 5 or more days to produce biogas.

Useful loading rates for cellulosic feedstock fermentation are described in detail elsewhere herein. For example, in certain embodiments: (i) the cellulosic feedstock comprises grass, the fermentation is aerobic, and the feedstock has a loading rate of between about 30 g/L and 50 g/L; (ii) the cellulosic feedstock comprises grass, the fermentation is anaerobic, and the feedstock has a loading rate of between about 30 g/L and 50 g/L; (iii) the cellulosic feedstock comprises guar gum, the fermentation is aerobic, and the feedstock has a loading rate of between 5 g/L and 15 g/L; or (iv) the cellulosic feedstock comprises guar gum, the fermentation is anaerobic, and the feedstock has a loading rate of between 1 g/L and 10 g/L.

Obtainable volatile fatty acid yields are described in detail elsewhere herein. For example, in certain embodiments the yield of VFAs is: (i) between about 35 mg/g and 125 mg/g per unit feedstock when the cellulosic feedstock comprises grass and the fermentation is aerobic; (ii) between about 70 mg/g and 175 mg/g per unit feedstock when the cellulosic feedstock comprises grass and the fermentation is anaerobic; (iii) between about 150 mg/g and 300 mg/g per unit feedstock when the cellulosic feedstock comprises guar gum and the fermentation is aerobic; or (iv) between about 400 mg/g and 600 mg/g per unit feedstock when the cellulosic feedstock comprises guar gum and the fermentation is anaerobic.

Certain embodiments are drawn to combining an inoculum comprising the cellulolytic microorganisms and the cellulosic feedstock to produce the fermentation solution. In certain embodiments, a nutrient solution is further added to the fermentation solution. Certain embodiments provide for a nutrient solution, a representative example of which is shown in Table 9.

In certain embodiments, the fermentation of the cellulosic feedstock occurs in a fermentation vessel.

Also provide herein is a carbon dependent nutrient removal and/or recovery process comprising the use of a volatile fatty acid produced by any of the fermentation of cellulosic feedstocks disclosed herein.

### EXAMPLES

The following examples demonstrate the concept of VFA generation from grass and guar gum. Provided are representative variables useful for VFA production; e.g., aerobic/anaerobic conditions, cellulase, temperature, and pH and determinations of VFA yield.

### Example 1

Initial samples were collected on 9/14 from three sources and submitted for background analysis. These samples were: municipal tap water; water reclamation plant effluent (Stickney Water Reclamation Plant (SWRP) operated by the Metropolitan Water Reclamation District of Chicago); and digester draw (SWRP operated by the Metropolitan Water Reclamation District of Chicago). Freshly mowed grass was collected on 9/18 in two 40-gallon garbage bags. Five reactors were set up with 1,500 mL liquid volumes (9/18) as shown below and then on 9/20, additional 500 mL liquid volumes were added to the first four reactors to accomplish the following configurations (Table 2).

TABLE 2

| Reactor | Setup | Note |
|---|---|---|
| One | Tap water (1500 mL) + (500 mL) | Additional volume of plant effluent |
| | and grass (100 g) | (500 mL) added on Sept. 20 |
| Two | Plant effluent (1500 mL) + (500 | Additional volume of plant effluent |
| | mL) and grass (100 g) | (500 mL) added on Sept. 20 |
| Three | Digester draw (1500 mL) + (500 | Additional volume of plant effluent |
| | mL) and grass (100 g) | (500 mL) added on Sept. 20 |
| Four | Digester draw (750 mL) + plant | Additional volume of plant effluent |
| | effluent (750 mL), plant effluent | (500 mL) added on Sept. 20 |
| | (250 mL) + Dig draw (250 mL), | |
| | and grass (100 g) | |
| Five | Digester draw (750 mL), plant | |
| | effluent (750 mL), and plant extract | |
| | (38.8276 g) | |

First set of samples were collected on 9/21 at about 3 pm and submitted on 9/25 due to Ortho-P 48 analysis hours holding time. On 9/26, additional samples were collected from each of the five reactors and submitted solely for VFA analysis. A comparison was also done using HACH's Volatile Acids test kit (TNT plus 872, Method 10240).

The five reactors were subsequently cleaned on 9/26 upon collecting samples. The reactor configuration was redesigned and four were filled on 9/28 (12 pm) and sample collected (Table 3 and Table 4):

TABLE 3

| Reactor | One | Two | Three¹ | Four² |
|---|---|---|---|---|
| Setup | Tap water | Plant effluent | Digester draw | Digester draw |
| | (3000 mL) and | (3000 mL) and | (1500 mL), plant | (1500 mL), plant |
| | grass (100 g) | grass (100 g) | effluent (1500 mL), | effluent (1500 mL), |
| | | | and grass (100 g) | and plant extract (25 g) |

¹Results for VFA and sCOD for these samples (reactor three) to be multiplied by two to account for the dilution that was performed with the exception of sample for the VFA parameter (no dilution was performed).
²Results for VFA and sCOD for these samples (reactor four) to be multiplied by five to account for the dilution that was performed.

TABLE 4
Collection (Date; Time)

9/28; 2:30 pm
9/29; 10 am
9/29; 12:30 pm
9/29; 3 pm
10/2; 3 pm
10/3; 3 pm
10/4; 3 pm
10/5; 3 pm
10/6; 3 pm

The four reactors were subsequently emptied of all liquid (10/6; 3 pm), leaving only solid material in each reactor. New SWRP effluent and digester draw source samples were collected at 3 pm on 10/11 (from the same locations as described above), as additional material was needed to fill the reactors.

The reactor configuration was redesigned and four were filled on 10/12 (9:45 am) and samples collected (Table 5 and Table 6).

TABLE 5

| Reactor | One | Two | Three¹ | Four² |
|---|---|---|---|---|
| Setup | Tap water | Plant effluent | Digester draw | Digester draw |
| | (3000 mL), grass | (3000 mL), grass | (1500 mL), plant | (1500 mL), plant |
| | (100 g), and | (100 g), and | effluent | effluent |
| | cellulase (3 g) | cellulase (3 g) | (1500 mL), grass | (1500 mL), plant |
| | | | (100 g), and | extract (25 g), and |
| | | | cellulase (3 g) | cellulase (3 g) |

¹Results for VFA and sCOD for these samples (reactor three) to be multiplied by two to account for the dilution that was performed.
²Results for VFA and sCOD for these samples (reactor four) to be multiplied by five to account for the dilution that was performed.

TABLE 6
Collection (Date; Time)

10/12; 12:45 pm
10/12; 3:45 pm
10/13; 9 am
10/13; 12 pm
10/13; 3 pm
10/16; 12:45 pm
10/17; 12:45 pm

On 10/17 at 12:45 pm, the squeezed grass mass VFA were measured using only HACH method.

WASSTRIP® Evaluation: On 10/18, approximately 10 gallons of mixed liquor sample was collected from the end of tank four of Battery D at the SWRP. A portion was submitted for laboratory analysis. The remaining mixed liquor sample was then allowed to settle for a period of 70 minutes and the supernatant was discarded. A portion of the thickened sludge sample was then submitted for laboratory analysis. At 8:35 am the following day, 3,600 mL of thickened sludge was combined with 1,090 mL of liquid from Reactor 3 and 260 mL of liquid from Reactor 2. Samples were taken at regular intervals (Table 7).

TABLE 7
Date and Time of Sampling

10/19; 8:35 am
10/19; 9:35 am
10/19; 10:35 am
10/19; 11:35 am
10/19; 12:35 pm
10/19; 1:35 pm
10/19; 2:35 pm
10/20; 8:35 am

### Results.

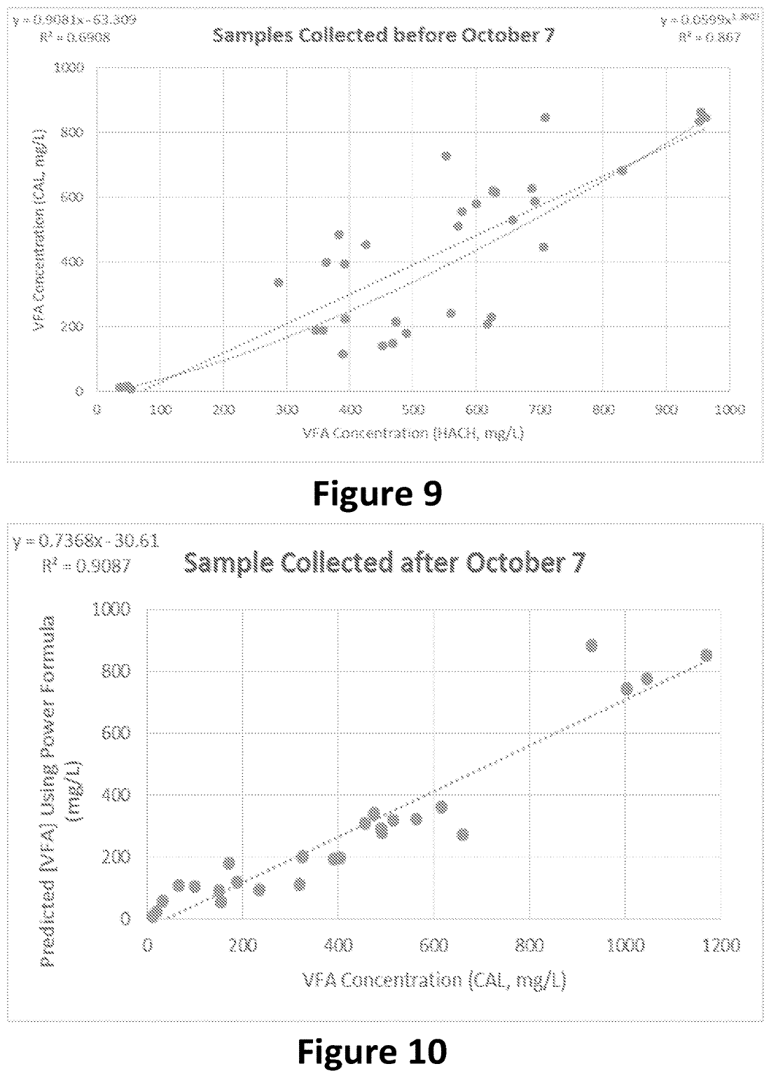

A total of 35 samples were taken from reactors one through four between 9/28 and 10/6. These samples were analyzed for VFA concentrations using both HACH's TNTplus 872 reagents (method 10240) and the services of the Calumet Analytical Laboratory. These concentrations were plotted (FIG. 9), along with linear and power trend lines, as well as the equation and R² value for each.

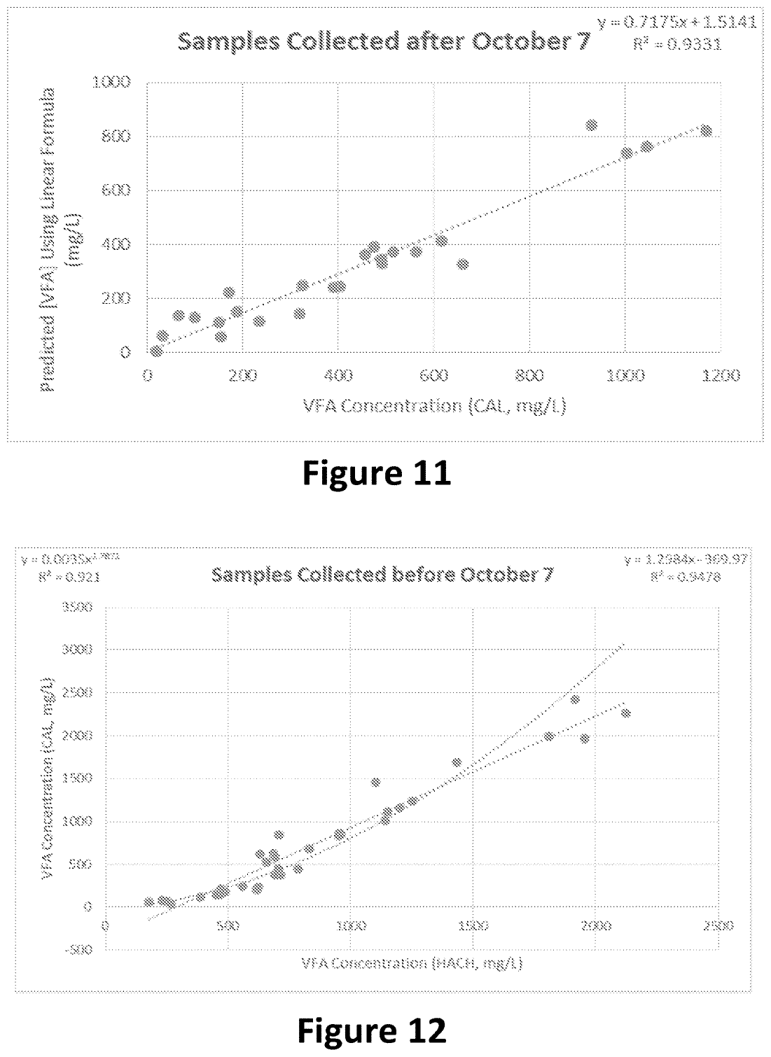

Predicted values, using the above formulas in FIG. 9 and obtained VFA concentrations using HACH method 10240 (x-axis values), were then developed for samples collected after 10/7 (total of 26 samples collected between 10/12 and 10/17). These predicted values were then plotted against the actual VFA concentration values returned by the Calumet Analytical Laboratory (FIG. 10 and FIG. 11).

Both the power and linear predictive formulas return a strong correlation to the Calumet Analytical Laboratory's VFA concentration (R² values of 0.9087 and 0.9331, respectively). These formulas were then corrected so that the resulting formula slope equals 1 (e.g. the predicted value of 600 mg/L, using the HACH method results, corresponds to the Calumet Analytical Laboratory values of 600 mg/L). The corrected predicted formula, and associated plots are shown in FIG. 10 and FIG. 11.

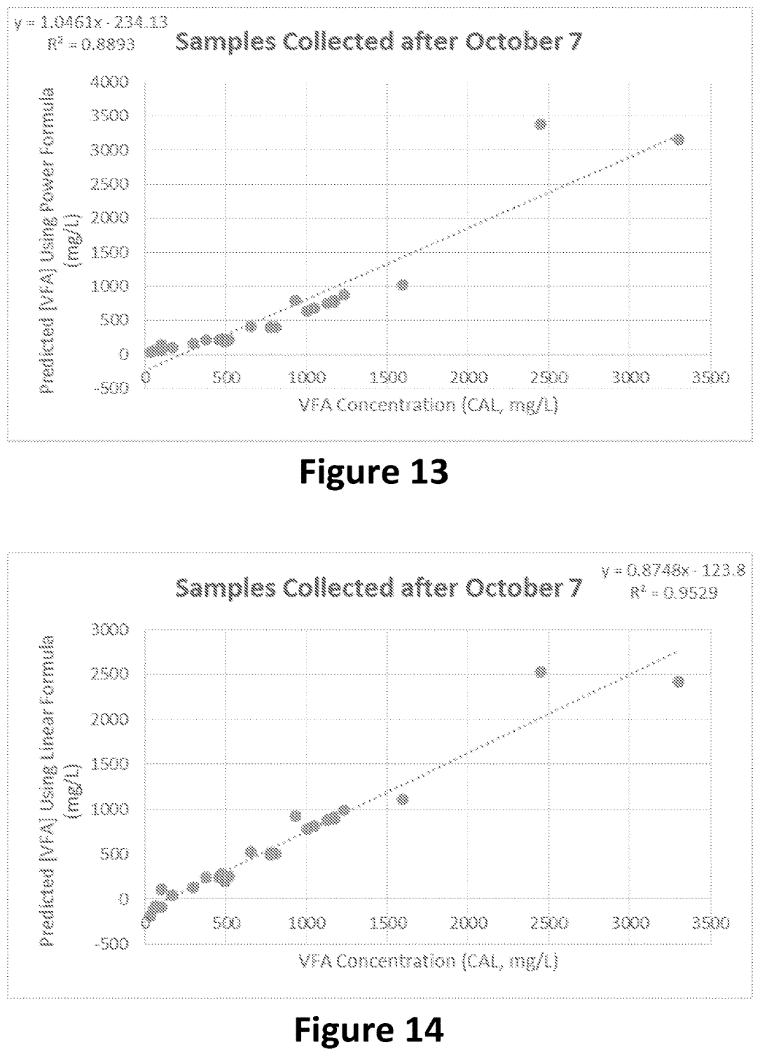

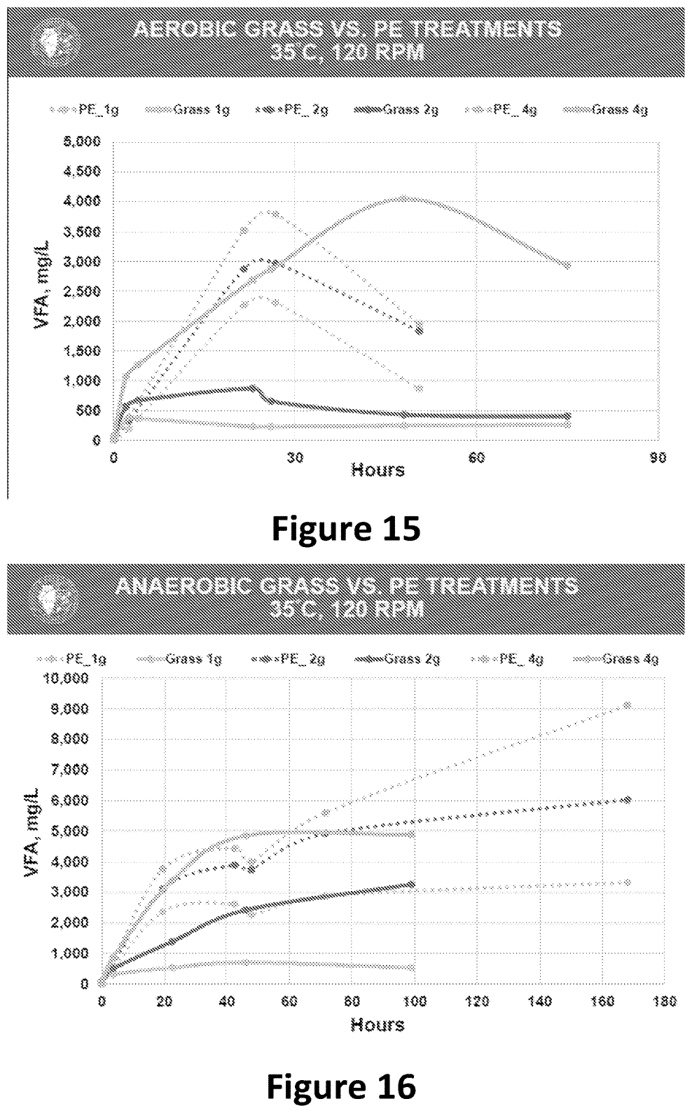

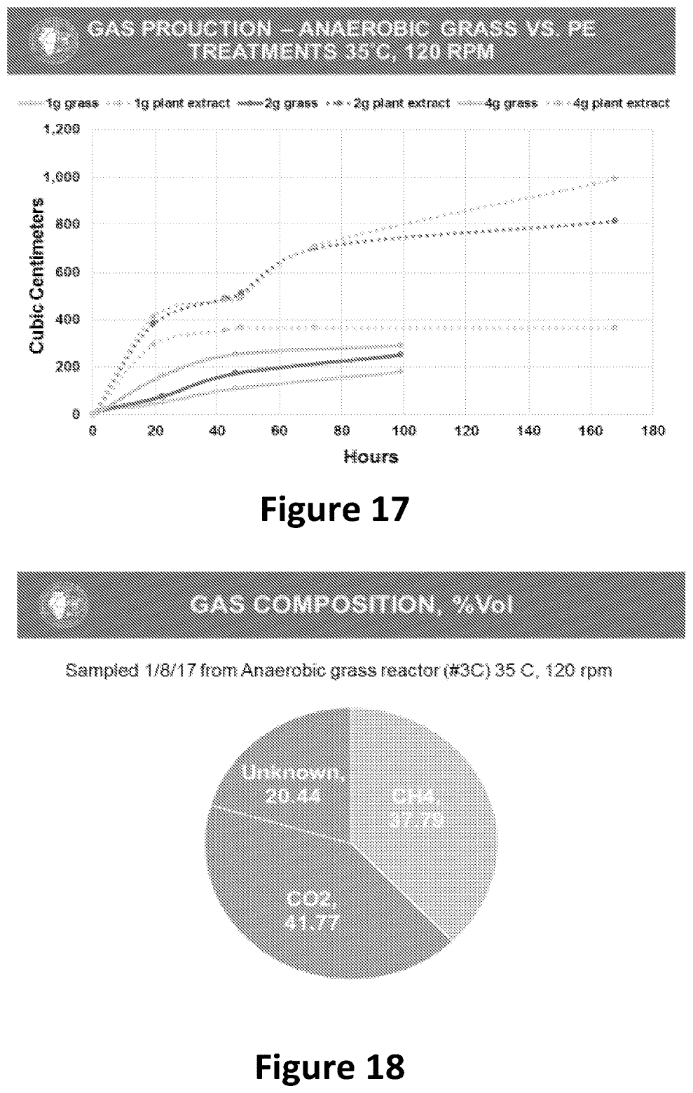

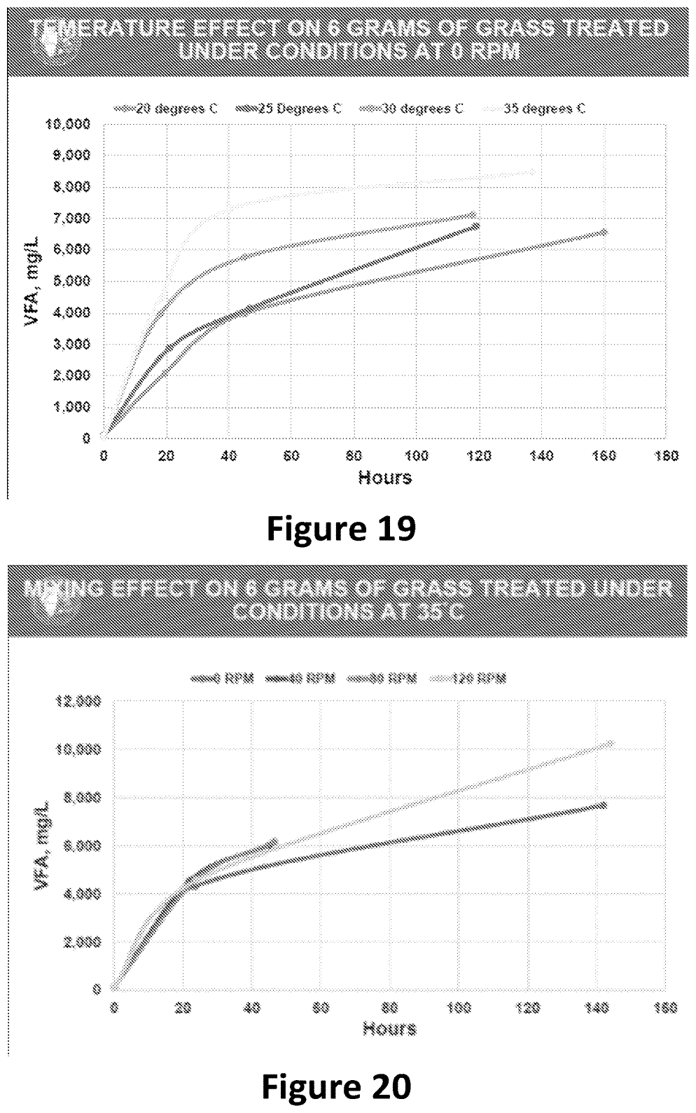

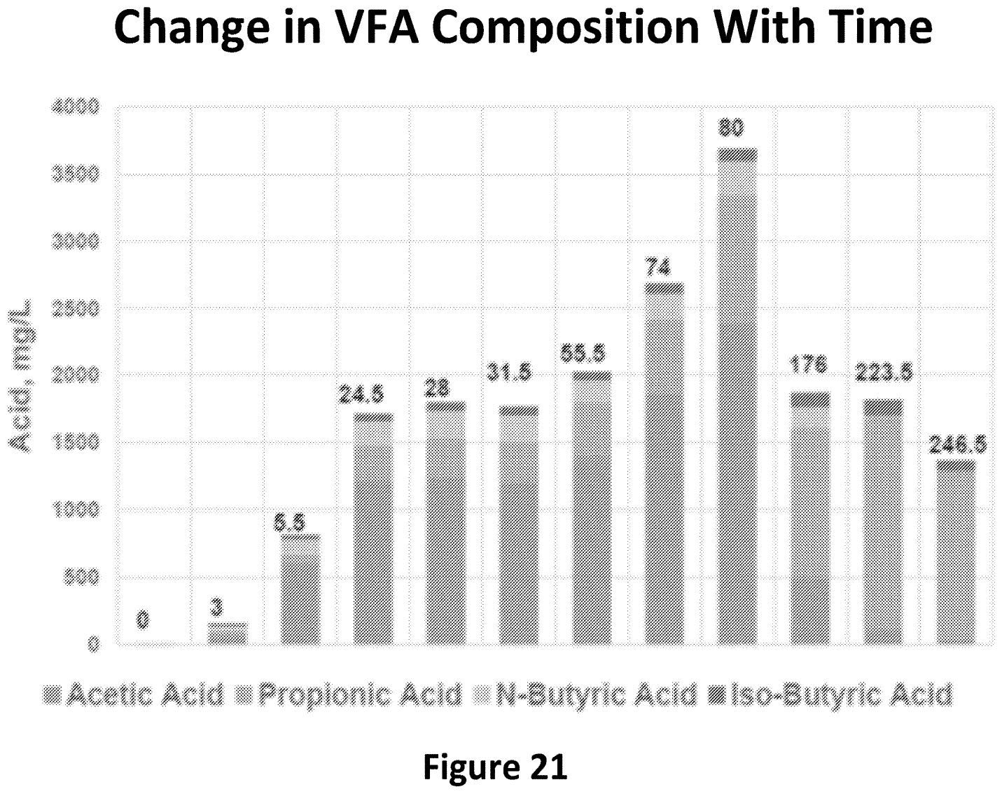

When samples were taken from reactors 3 and 4, these samples were diluted so that they could be prepared for VFA and soluble COD analysis, which require filtering through a 0.45 micron filter. The predictive analysis, described above, uses the raw values obtained before multiplying the sample by the appropriate dilution factor. The graphs of these multiplied results are illustrated in FIG. 12, FIG. 13, and FIG. 14.

The predictive formula can be adjusted in order to correct the resulting trendline slope to 1 (FIG. 13 and FIG. 14).

### Example 2

### 1. Source of Grass and Guar Gum Feeding and Degrading Cellulolytic Organisms.

The seed source reservoir of microorganisms was municipal anaerobic digesters (municipal wastewater treatment plant operated at a temperature of 95 degrees Fahrenheit and hydraulic/sludge retention time ranging from about 20 to 30 days). Other sources may be cow and buffalo dung and soil.

### 2. Test Procedure for Derivation, Concentration and Maintenance of Grass and Guar Gum Feeding and Degrading Cellulolytic Organisms.

A. Derivation of Cellulolytic Organisms:

On 10/12, two aerobic reactors (grass and guar gum) with lids having vent line were established and operated as shown below in order to allow the organisms within these reactors to adapt to the fed substrates and mature for over a month (11/17).

Oxygen: Not aerated but exposed to atmosphere (aerobic).

Mixing: Only before sampling; occasional mixing unobjectionable.

Temperature: 25 to 30±1 C. pH: 7 to 7.5±0.3 pH units.

### Grass:

The first (Reactor 3), consisted of 1,500 mL digester draw, 1,500 mL of SWRP effluent, 100 g of dried shredded grass, and 3 g cellulase (1 g/l L).

### Guar Gum:

The second (Reactor 4) consisted of 1,500 mL digester draw, 1,500 mL of SWRP effluent, 25 g of plant extract (PE; guar gum), and 3 g cellulase (i.e. 1 g/l L).

B. Process for Cultivation and Concentration of Cellulolytic Organisms.

In order to further cultivate and concentrate derived cellulolytic organisms in aerobic and anaerobic conditions, two stock fermenters for each substrate were set up using the liquid portion from the two reactors mentioned in the previous step (those two reactors were labeled as Reactors 3 and 4 in derivation step) as shown below. To accommodate large feed stock volume, 11 L volume was split into two 5.5 L reactors (3A and 3B and likewise 4A and 4B).

Aerobic Grass Fermenter 3A: Placed 250 mL of old Reactor 3 liquid and mixed with 2,625 mL of digester draw and 2,625 mL of effluent. Total volume of 5.5 L.

Aerobic Grass Fermenter 3B: Setup same as 3A, above. Total volume of 5.5 L.

Anaerobic Grass Fermenter 3C: Placed 500 mL of old Reactor 3 liquid into fermenter and mixed with 5,250 mL of digester draw and 5,250 mL of effluent. Total volume of 11 L.

Aerobic Guar Gum Fermenter 4A: Placed 250 mL of old Reactor 4 liquid and mixed with 2,625 mL of digester draw and 2,625 mL of effluent. Total volume of 5.5 L.

Aerobic Guar Gum Fermenter 4B: Setup same as 4A, above.

Anaerobic Guar Gum Fermenter 4C: Placed 500 mL of old Reactor 4 liquid into fermenter and mixed with 5,250 mL of digester draw and 5,250 mL of effluent. Total volume of 11 L.

C. Cultivation, Concentration and Maintenance of Cellulolytic Organisms.

A portion of fermenter content was removed and replenished with equal portion of salt-vitamin solution with grass in fermenters 3A, 3B, and 3C and equal portion of salt-vitamin solution with guar gum in fermenters 4A, 4B, and 4C, approximately twice per week as shown in Table 8 below. Occasionally, left over biomass from serum bottles were also fed back into reactors to conserve the mass of organisms. However, such left over additions are not needed.

One deterministic factor for servicing the fermenters is the length of time for which stock cultures of the vibrant cellulolytic organisms need to be maintained. The cellulolytic organisms in lab fermenters have been observed to survive without servicing the fermenters as well but servicing the fermenters can be performed.

TABLE 8
Fermenter Fluid Extraction, Replenishment, and Feeding Schedule.

| Day Sample Removed | Amount (mL) | Replenishment Source |
|---|---|---|
| November 22 | 300 each from 3A, 3B, 4A, and 4B 600 each | Salt-Vitamin Solution |
| | from 3C and 4C | |
| November 24 | 175 each from 3A, 3B, 4A, and 4B 350 each | Salt-Vitamin Solution |
| | from 3C and 4C | |
| November 28 | 175 each from 3A, 3B, 4A, and 4B 350 each | Salt-Vitamin Solution |
| | from 3C and 4C | |
| December 1 | 175 each from 3A, 3B, 4A, and 4B 350 each | Salt-Vitamin Solution |
| | from 3C and 4C | |
| December 5 | 175 each from 3A, 3B, 4A, and 4B 350 each | Salt-Vitamin Solution |
| | from 3C and 4C | |
| December 8 | 175 each from 3A, 3B, 4A, and 4B 350 each | Salt-Vitamin Solution |
| | from 3C and 4C | |
| December 12 | 175 each from 3A, 3B, 4A, and 4B 350 each | Salt-Vitamin Solution |
| | from 3C and 4C | |
| December 15 | 175 each from 3A, 3B, 4A, and 4B 350 each | Salt-Vitamin Solution |
| | from 3C and 4C | |
| December 19 | 175 each from 3A, 3B, 4A, and 4B 350 each | Salt-Vitamin Solution |
| | from 3C and 4C | |
| December 21 | 175 each from 3A and 3B | Serum bottles from |
| | | experiment set up on 12/18 |
| | 175 each from 4A and 4B 350 each from 3C | Salt-Vitamin Solution |
| | and 4C | |
| December 26 | 175 each from 3A and 3B | Serum bottles from |
| | | experiment set up on 12/18 |
| | 175 each from 4A and 4B 350 each from 3C | Salt-Vitamin Solution |
| | and 4C | |
| December 29 | 175 each from 3A, 3B, 4A, and 4B 350 each | Salt-Vitamin Solution |
| | from 3C and 4C | |
| January 2 | 175 each from 3A, 3B, 4A, and 4B 350 each | Salt-Vitamin Solution |
| | from 3C and 4C | |
| January 5 | 175 each from 3A, 3B, 4A, and 4B 350 each | Salt-Vitamin Solution |
| | from 3C and 4C | |
| January 9 | 175 each from 3A, 3B, 4A, and 4B 350 each | Salt-Vitamin Solution |
| | from 3C and 4C | |
| January 12 | 175 each from 3A, 3B, 4A, and 4B 350 each | Salt-Vitamin Solution |
| | from 3C and 4C | |
| January 16 | 175 each from 4A and 4B | Serum bottles from |
| | | experiment set up on 1/9 |
| | 175 each from 3A and 3B 350 each from 3C | Salt-Vitamin Solution |
| | and 4C | |
| January 19 | 175 each from 3A, 3B, 4A, and 4B 350 each | Salt-Vitamin Solution |
| | from 3C and 4C | |
| January 23 | 350 from 4C | Serum bottles from |
| | | experiment set up on 1/16 |
| | 175 each from 3A, 3B, 4A, and 4B 350 each | Salt-Vitamin Solution |
| | from 3C | |
| January 26 | 175 each from 3A, 3B, 4A, and 4B 350 each | Salt-Vitamin Solution |
| | from 3C and 4C | |
| January 30 | 175 each from 3A, 3B, 4A, and 4B 350 each | Salt-Vitamin Solution |
| | from 3C and 4C | |
| February 2 | 175 each from 3A, 3B, 4A, and 4B 350 each | Salt-Vitamin Solution |
| | from 3C and 4C | |
| February 6 | 175 each from 3A, 3B, 4A, and 4B 350 each | Salt-Vitamin Solution |
| | from 3C and 4C | |
| February 13 | 175 each from 3A, 3B, 4A, and 4B 350 each | Salt-Vitamin Solution |
| | from 3C and 4C | |
| February 20 | 175 each from 3A, 3B, 4A, and 4B 350 each | Salt-Vitamin Solution |
| | from 3C and 4C | |
| February 22 | 175 each from 3A, 3B, 4A, and 4B 350 each | Salt-Vitamin Solution |
| | from 3C and 4C | |

On all of the dates listed above, the fermenters were fed using the following materials:

Fermenters 3A/3B: 11 g each of grass (2 g/L substrate feed rate)

Fermenter 3C: 22 g of grass at a rate of 2 g per liter (2 g/L substrate feed rate)

Fermenters 4A/4B: 0.55 g plant extract (0.1 g/L substrate feed rate)

Fermenter 4C: 1.1 g plant extract at a rate of 0.1 g per liter (0.1 g/L substrate feed rate)

Additionally, the fermenters were restocked with a volume of salt-vitamin solution equivalent to what was extracted, except when noted otherwise. A representative recipe for the salt-vitamin solution is included as shown in Table 9.

D. Monitoring of Fermenters for Healthy Cellulolytic Organisms.

Fermenters were monitored for proper operating conditions to ensure healthy growth and maintenance of a consortium of cellulolytic organisms' population. The following parameters were monitored with frequencies as follows: (i) measured pH and temp twice a week, Tuesdays and Fridays (measured twice per week) and (ii) four drawn samples from fermenters were composited (over two weeks) and analyzed for chemical oxygen demand (COD), total solids (TS), volatile solids (VS), volatile fatty acids (VFA). pH and VFAs were used as indicators for “sour” reactors. The preceding procedure can be used for the grass and guar gum degradation process start-up.

### 3. Use of Cellulase for Aerobic or Anaerobic Cellulolytic Organisms.

Cellulase was added at 1 g per liter dose to all four reactors in a side-by-side testing to evaluate the impact of cellulase on VFA concentrations after a reaction time of about 2 to 4 days.

Means of populations of VFA concentrations of all four grass reactors with and without cellulase had no statistically significant difference. However, relating these concentrations with reaction time revealed a significantly different outcome, i.e., addition of cellulase reduced time to achieve the same concentration compared to omission of cellulase, indicating that cellulase did not affect final VFA production quantity, but it increased the reaction rate. Benefits of cellulase, despite its cost, include: (i) good for rapid stock culture growth; (ii) good for quicker VFA production on an emergency basis; and (iii) regain the strength of cellulolytic organisms from a toxic upset in the reactor.

A reduction in initial derivation time can be claimed due to cellulase use; cellulase enzyme promotes the outgrowth of cellulolytic organisms and thereby expedites the initial derivation time. Another claim can be the increased reaction rate with cellulase use, which reduces maximum VFA production time in the VFA production reactor, while end quantity of VFA is not impacted.

### 4. Controlled Laboratory Reactor/Fermenter Conditions for Cultivation, Concentration and Maintenance of Grass and Guar Gum Feeding Cellulolytic Organisms (Steps 2b and 2c).

The purpose of maintaining and monitoring for certain operating conditions is to promote the growth of grass and guar gum feeding cellulolytic organisms to produce stock cultures and maintain young and dynamic biomass in the reactor for experiments in laboratory or to run field-scale grass and/or guar gum degradation fermenter systems.

Aerobic Grass Fermenter 3A/3B.

Oxygen: Not aerated but exposed to atmosphere (aerobic)

Mixing: Only before sampling

Temp.: 25 to 30±1 C

pH: 7 to 7.5±0.3 pH units

Anaerobic Grass Fermenter 3C.

Oxygen: No oxygen (anaerobic)

Mixing: Only before sampling

Temp.: 25 to 30±1 C

pH: 7 to 7.5±0.3 pH units

Aerobic Guar Gum Fermenter 4A/4B:

Oxygen: Not aerated but exposed to atmosphere

Mixing: Only before sampling

Temp.: 25 to 30±1 C

pH: 7 to 7.5±0.3 pH units

Anaerobic Guar Gum (#437) Fermenter 4C:

Oxygen: No oxygen (anaerobic)

Mixing: Only before sampling

Temp.: 25 to 30±1 C

pH: 7 to 7.5±0.3 pH units

### 5. Pre-Treatment of Grass Feed Stock Before Use.

Grass was dried at room temperature from 68% down to 5 to 10% moisture content and then pulverized to millimeter size particles before feeding to the reactors.

Guar gum in powder form was dissolved in lukewarm water before feeding to avoid undissolved lumps seating unutilized in the reactors.

### 6. Serum Bottle Test Procedures.

A. 3A/3B at 35° C. and 120 RPM (12/18).

Consisted of 17 serum bottles (Table 10)—triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (1 g); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The 425 mL inoculum extracted from each fermenter was replaced by 50% salt-vitamin solution, 25% digester draw, and 25% SWRP effluent.

TABLE 10
Serum bottle experimental design for grass as a feedstock.

| | | Salt- | | | | Total |
|---|---|---|---|---|---|---|
| | Inoculum | Vitamin | | Glucose | Sodium Acetate | Liquid |
| | Draw | Solution | Grass | Solution | Solution | Volume |
| | (mL) | (mL) | (g) | (mL, 0.065 g/L) | (mL, 1 g/L) | (mL) |
| Blank | 50 | 50 | 0 | 0 | 0 | 100 |
| (Triplicate) | | | | | | |
| Control 1 | 50 | 30 | 0 | 20 | 0 | 100 |
| (Triplicate) | | | | | | |
| Control 2 | 50 | 30 | 0 | 0 | 20 | 100 |
| (Triplicate) | | | | | | |
| Test 1 | 50 | 50 | 1 | 0 | 0 | 100 |
| (Duplicate) | | | | | | |
| Test 2 | 50 | 50 | 2 | 0 | 0 | 100 |
| (Duplicate) | | | | | | |
| Test 3 | 50 | 50 | 4 | 0 | 0 | 100 |
| (Duplicate) | | | | | | |
| Test 4 | 50 | 50 | 6 | 0 | 0 | 100 |
| (Duplicate) | | | | | | |

| | | |
|---|---|---|
| TOTAL | 850 | Replaced in stock fermenters with 50% salt-vitamin solution, |
| 3A/3B: | | 25% digester draw, and 25% SWRP effluent. |

B. 3C at 35° C. and 120 RPM (1/2).

Consisted of 17 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (1 g); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The 850 mL inoculum extracted from the fermenter was replaced by 50% salt-vitamin solution, 25% digester draw, and 25% SWRP effluent. The setup configuration was the same as described in Table 10, but using fluid from Fermenter 3C, instead.

C. 4A/4B at 35° C. and 120 RPM (1/9).

Consisted of 17 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and plant extract (1 g); duplicate test bottles with 50 mL inoculum, 50 mL salt-vitamin solution, and plant extract (2 g); duplicate test bottles with 50 mL inoculum, 50 mL salt-vitamin solution, and plant extract (4 g); and duplicate test bottles with 50 mL inoculum, 50 mL salt-vitamin solution, and plant extract (0.5 g). The 425 mL inoculum extracted from each fermenter was replaced by 50% salt-vitamin solution, 25% digester draw, and 25% SWRP effluent. The setup configuration was the same as described in Table 11, but using fluid from Fermenter 4A/4B, instead.

TABLE 11
Serum bottle experimental design for guar gum as a feedstock.

| | | Salt- | | | Sodium | Total |
|---|---|---|---|---|---|---|
| | Inoculum | Vitamin | Guar | Glucose | Acetate | Liquid |
| | Draw | Solution | Gum | Solution | Solution | Volume |
| | (mL) | (mL) | (g) | (mL, 0.065 g/L) | (mL, 1 g/L) | (mL) |
| Blank | 50 | 50 | 0 | 0 | 0 | 100 |
| (Triplicate) | | | | | | |
| Control 1 | 50 | 30 | 0 | 20 | 0 | 100 |
| (Triplicate) | | | | | | |
| Control 2 | 50 | 30 | 0 | 0 | 20 | 100 |
| (Triplicate) | | | | | | |
| Test 1 | 50 | 50 | 1 | 0 | 0 | 100 |
| (Duplicate) | | | | | | |
| Test 2 | 50 | 50 | 2 | 0 | 0 | 100 |
| (Duplicate) | | | | | | |
| Test 3 | 50 | 50 | 4 | 0 | 0 | 100 |
| (Duplicate) | | | | | | |
| Test 4 | 50 | 50 | 0.5 | 0 | 0 | 100 |
| (Duplicate) | | | | | | |

| | | |
|---|---|---|
| TOTAL | 850 | Replaced in stock fermenters with 50% salt-vitamin solution, |
| 4A/4B: | | 25% digester draw, and 25% SWRP effluent. |

D. 4C at 35° C. and 120 RPM. (1/16).

Consisted of 17 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and plant extract (1 g); duplicate test bottles with 50 mL inoculum, 50 mL salt-vitamin solution, and plant extract (2 g); duplicate test bottles with 50 mL inoculum, 50 mL salt-vitamin solution, and plant extract (4 g); and duplicate test bottles with 50 mL inoculum, 50 mL salt-vitamin solution, and plant extract (0.5 g). The setup configuration was the same as described in Table 11, but using fluid from Fermenter 4C, instead.

i. Serum Bottle Temperature Variable Experiment Setup:

A. 3C at 20° C. and 0 RPM (1/24).

Consisted of 15 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The 750 mL inoculum extracted from the fermenter was replaced by 50% salt-vitamin solution, 25% digester draw, and 25% SWRP effluent.

TABLE 12
Serum bottle experimental design for grass
as a feedstock with varying temperature.

| | | Salt- | | | Sodium | Total |
|---|---|---|---|---|---|---|
| | Inoculum | Vitamin | | Glucose | Acetate | Liquid |
| | Draw | Solution | Grass | Solution | Solution | Volume |
| | (mL) | (mL) | (g) | (mL, 0.065 g/L) | (mL, 1 g/L) | (mL) |
| Blank | 50 | 50 | 0 | 0 | 0 | 100 |
| (Triplicate) | | | | | | |
| Control 1 | 50 | 30 | 0 | 20 | 0 | 100 |
| (Triplicate) | | | | | | |
| Control 2 | 50 | 30 | 0 | 0 | 20 | 100 |
| (Triplicate) | | | | | | |
| Test 2 | 50 | 50 | 2 | 0 | 0 | 100 |
| (Duplicate) | | | | | | |
| Test 3 | 50 | 50 | 4 | 0 | 0 | 100 |
| (Duplicate) | | | | | | |
| Test 4 | 50 | 50 | 6 | 0 | 0 | 100 |
| (Duplicate) | | | | | | |

| | | |
|---|---|---|
| TOTAL | 750 | Replaced in stock fermenters with 50% salt-vitamin solution, |
| 3C: | | 25% digester draw, and 25% SWRP effluent. |

B. 3C at 35° C. and 0 RPM (1/24).

Consisted of 15 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The 750 mL inoculum extracted from the fermenter was replaced by 50% salt-vitamin solution, 25% digester draw, and 25% SWRP effluent. The setup configuration was the same as described in Table 12.

C. 3C at 25° C. and 0 RPM (1/31).

Consisted of 15 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The setup configuration was the same as described in Table 12; however, the 750 mL inoculum extracted from the fermenter was replaced by the discarded solution from the serum bottle experiment set up on 1/24.

D. 3C at 30° C. and 0 RPM (1/31).

Consisted of 15 serum bottles-triplicate blank bottles with 50 mL inoculum and 50 mL salt-vitamin solution; triplicate control bottles with 50 mL inoculum, 30 mL salt-vitamin solution, and 20 mL dextrose (0.065 g/L); triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (2 g); duplicate test bottles with 50 mL inoculum, 50 mL salt-vitamin solution, and grass (4 g); and duplicate test bottles with 50 mL inoculum, 50 mL salt-vitamin solution, and grass (6 g). The setup configuration was the same as described in Table 12; however, the 750 mL inoculum extracted from the fermenter was replaced by the discarded solution from the serum bottle experiment set up on 1/24.

ii. Serum Bottle Mixing Speed Variable Experiment Setup.

A. 3C at 35° C. and 40 RPM (2/5).

Consisted of 10 serum bottles-triplicate blank bottles with 50 mL inoculum and 50 mL salt-vitamin solution; triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (4 g); and duplicate test bottles with 50 mL inoculum, 50 mL salt-vitamin solution, and grass (6 g). The 500 mL inoculum extracted from the fermenter was replaced by the discarded solution from the serum bottle experiment set up on 1/31.

TABLE 13
Serum bottle experimental design for grass
as a feedstock with varying mixing speed.

| | | Salt- | | | Sodium | Total |
|---|---|---|---|---|---|---|
| | Inoculum | Vitamin | Guar | Glucose | Acetate | Liquid |
| | Draw | Solution | Gum | Solution | Solution | Volume |
| | (mL) | (mL) | (g) | (mL, 0.065 g/L) | (mL, 1 g/L) | (mL) |
| Blank | 50 | 50 | 0 | 0 | 0 | 100 |
| (Triplicate) | | | | | | |
| Control 1 | 50 | 30 | 0 | 20 | 0 | 100 |
| (Triplicate) | | | | | | |
| Control 2 | 50 | 30 | 0 | 0 | 20 | 100 |
| (Triplicate) | | | | | | |
| Test 2 | 50 | 50 | 2 | 0 | 0 | 100 |
| (Duplicate) | | | | | | |
| Test 3 | 50 | 50 | 4 | 0 | 0 | 100 |
| (Duplicate) | | | | | | |
| Test 4 | 50 | 50 | 0.5 | 0 | 0 | 100 |
| (Duplicate) | | | | | | |

| | | |
|---|---|---|
| TOTAL | 500 | Replaced in stock fermenters with the discarded solution from |
| 3C: | | the serum bottle experiment set up on 1/31. |

B. 3C at 35° C. and 80 RPM (2/5).

Consisted of 10 serum bottles-triplicate blank bottles with 50 mL inoculum and 50 mL salt-vitamin solution; triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (4 g); and duplicate test bottles with 50 mL inoculum, 50 mL salt-vitamin solution, and grass (6 g). The 500 mL inoculum extracted from the fermenter was replaced by the discarded solution from the serum bottle experiment set up on 1/31. The setup configuration was the same as described in Table 13.

C. 3C at 35° C. and 0 RPM (2/7).

Consisted of 10 serum bottles-triplicate blank bottles with 50 mL inoculum and 50 mL salt-vitamin solution; triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (4 g); and duplicate test bottles with 50 mL inoculum, 50 mL salt-vitamin solution, and grass (6 g). The setup configuration was the same as described in Table 13; however, the 500 mL inoculum extracted from the fermenter was replaced by the discarded solution from the serum bottle experiment set up on 2/5.

D. 3C at 35° C. and 120 RPM (2/7).

Consisted of 10 serum bottles-triplicate blank bottles with 50 mL inoculum and 50 mL salt-vitamin solution; triplicate control bottles with 50 mL inoculum, 30 mL salt-vitamin solution, and 20 mL sodium acetate (1 g/L); duplicate test bottles with 50 mL inoculum, 50 mL salt-vitamin solution, and grass (4 g); and duplicate test bottles with 50 mL inoculum, 50 mL salt-vitamin solution, and grass (6 g). The setup configuration was the same as described in Table 13; however, the 500 mL inoculum extracted from the fermenter was replaced by the discarded solution from the serum bottle experiment set up on 2/5.

### 7. VFA Measurement Methods.

VFAs were measured following HACH procedures by the HACH Company (e.g., Volatile Acids. DOC316.53.01259. Esterification Method. Method 10240. 50 to 2500 mg/L CH₃COOH (Acetic Acid). TNTPLUS™ 872. Scope and application: For digested sludges, activated sludges, process water and food products. 1 Adapted from The Analyst, 87, 949 (1962)). Additionally, many samples that were analyzed by HACH method were also analyzed by District's Analytical Laboratory Division (ALD) labs using GC-FID method.

### 8. Procedure for Determination of VFA Composition.

District's ALD method was used to determine only four out of 16 components of VFAs, namely: acetic acid; propionic acid; iso-butyric acid; and N-butyric acid.

### 9. Gas Collection Procedure and Gas Flowrate and Composition Measurement Method.

The gas produced in each treatment described above was extracted from serum bottles with graduated glass syringe and collected in separate Tedlar bags by treatment. Total gas production over a defined time period was recorded and used for the determination of total gas production and gas production rate per unit time.

For a bigger scale (6-L scale-up from serum bottle volume), the gas production was measured by Ritter milli gas counter, a volumetric gas flow meter (world wide web at www.ritter.de) and recorded with Rigamo (v. 3.1) software. The in-built feature was used to determine total gas production and gas flow rates.

Two major ingredients (CH₄ and CO₂) were measured in the gas samples using Gas Chromatographer SRI 8610C. The instrument was calibrated using known concentrations of both reference gas standards; the reference gas with known concentrations were diluted to different concentrations for calibration purpose. The gas was injected into an instrument for analysis upon calibration.

Average CH₄ and CO₂ were 37.79 and 41.77%, respectively.

### 10. Procedure for Identification of Celluloytic Organisms Using Genetic Analysis.

On 1/30, 150 mL content was removed from each fermenter 3C and 4C (and 75 mL from each fermenter 3A, 3B, 4A, and 4B) to submit for microbial identification. The 16S amplicon sequencing data analysis techniques was used to identify and examine the celluloytic microbial community in 4 samples from all four treatments. This technique provides taxonomic annotation and evidence of the presence of the functional gene; however, it can't identify organisms down to species level. It may be possible to infer functional genes from the 16S.

The 16S amplicon technique identified that all samples are highly diverse at the “species” level with roughly 500 taxa observed per sample, i.e., at least 500 dominant organisms in each of the 4 reactors.

The method using 16S amplicon sequence data is only able to detect bacterial functional genes. It is not possible to detect fungi or non-bacterial functional genes from the sample, as the 16S gene is only present in bacteria. It is theoretically possible to detect archaea using the 16S primers, however the typical primers are more specific to bacteria and the bacterial sequence data would likely overshadow or obscure any archaeal sequences that are present.

A shotgun metagenomics approach may be used to identify broader functional genes of cellulolytic organisms. It will detect the actual functional genes that are present in the samples as well as organisms from all domains of life, i.e. bacteria, archaea, eukaryotes, and viruses. However, it is less sensitive with respect to 16S amplicon sequencing.

### 11. Identification of Dominant Workhorse Organisms.

As stated above, the samples were analyzed to identify predominant cellulolytic organisms by their taxon names.

12. Procedure for Boosting Efficiency of VFA Production from the Grass and Guar Gum Feed Stocks.
a. Grass was pulverized to increase its surface area exposure to organisms and thereby to increase VFA and gas production efficiency. This step will not only reduce VFA/gas production time, but also help prevent clogged tubing in laboratory reactors and piping and pumps in large scale reactors.
b. Use of salt-vitamin water acted as catalyst in terms of supplying the needed macro and micronutrients to sustain dynamic population of organisms and thus, maintaining process efficiency.
c. Use of cellulase at 1 g per one-liter liquid volume.
d. In this representative example for VFA production a pH of 6.0 to 6.5 is ideal; but for gas production, pH should not fall below 6.5.

### 13. Determination of Times Needed for Maximum VFAs and Gas Productions.

VFA concentrations and gas productions measured at different times were plotted against respective times. The time taken to reach a highest peak (i.e., highest VFA concentration or gas production) was noted as the required time for maximizing VFA and gas productions. An inflection point determined if both graphs were superimposed on one chart, may also be used as maximum allowable time for VFA production, as VFA are used as precursors to gas production by methanogens.

For maximizing gas production, pH adjustment may be needed using sodium bicarbonate or sodium or potassium hydroxide if the reactor pH drops falls below 6.5. Conversely, a pH drop below 6.5 would suppress methane producing organisms and allow VFA concentrations to accumulate in the reactor.

### 14. Determination of VFA and Gas Production Per Unit Feedstock by Weight.

For each treatment, total VFA mass was calculated by multiplying highest concentration with total liquid volume in the reactor. The total gas production measured for each treatment was recorded. Total mass of VFAs was divided by the original amount of substrate added to the reactor at the start of experiments. Likewise, total volumetric gas production was divided by the original amount of substrate added at the start of the experiments. This method will determine VFA and gas production per unit weight of feedstock.

### 15. Procedure for Evaluating Compatibility of Grass and Guar Gum Feed Stocks Derived VFAs for the Waste Activated Sludge Stripping to Remove Internal Phosphorus (WASSTRIP) Process.

WASSTRIP® Evaluation (10/18; 2:12 pm). Approximately 10 gallons of mixed liquor sample was collected from the end of tank four of Battery D at the SWRP. A portion was submitted for laboratory analysis under LIMS number 8074350 for baseline TS, TVS, and Ortho-P. The remaining mixed liquor sample was then allowed to settle for a period of 70 minutes (settling criterion was developed during previous unrelated testing) and the supernatant was discarded. A portion of the thickened sludge sample was then submitted for laboratory analysis under LIMS number 8074351 for baseline TS, TVS, and Ortho-P. At 8:35 am the following day, 3,600 mL of thickened sludge was combined with 1,090 mL of liquid from Reactor 3 (grass derived VFA) and 260 mL of liquid from Reactor 2 (guar gum derived VFA) and a portion was analyzed for TP and Ortho-P. Samples were taken at regular intervals for Ortho-P analysis, as shown in Table 14.

TABLE 14

| Date and Time of Sampling | Hours Since Initial Setup |
|---|---|
| 10/19, 8:35am | 0 |
| 10/19, 9:35am | 1 |
| 10/19, 10:35am | 2 |
| 10/19, 11:35am | 3 |
| 10/19, 12:35pm | 4 |
| 10/19, 1:35pm | 5 |
| 10/19, 2:35pm | 6 |
| 10/20, 8:35am | 24 |

With respect to initial TP of the mixture of WAS and VFA additions, the percent ortho-P were calculated at above hours since initial setup. The ortho-P concentrations of WAS, thickened WAS upon 70 minutes of settling, and the mixture of WAS and VFA additions were used as reference or background concentrations in the percent ortho-P calculations. 16. Applications of gas (derived from grass and guar gum feed stocks) in wastewater treatment operations.

It is contemplated that digester gas may be separated into two major gas streams if desired; CH₄ and CO₂ before its use.

Methane, preferably but not necessarily upon separation, may be used in boiler for heat energy production, or in CHP systems to produce heat and electricity, or to supply to natural gas lines while carbon dioxide may be sparged in digester draw to reduce its pH for reducing struvite formation and improving biosolids dewatering performance, or producing a food grade quality gas for carbonation for soft drinks.

Before or after gas separation, it may be used for the production of heat energy upon burning methane portion in the boilers, which in turn, can be used for the following:

a. Production of hot water;

b. Production of steam;

c. Heating and cooling buildings;

d. Raising temperature of digester feed; and

e. Maintaining digester's operating temperature.

### Example 3

### Next-Generation Amplicon Sequencing—UIC Research Resources Center

This Example provides a summary of the basic bioinformatic analysis included with the amplicon sequencing services provided by the UIC Research Resources Center (RRC). The end result of these bioinformatics services is to provide investigators basic information concerning the abundance of taxa present in the samples. The basic bioinformatic analysis includes basic processing of raw sequence data using QIIME; including read merging, adapter & quality trimming, and chimeric checking; to generate a table of operational taxonomic units (OTU) with abundance data and associated taxonomic annotations (J. G. Caporaso, et al. Qiime allows analysis of high-throughput community sequencing data. Nature methods, 7(5):335-336, May 2010). Sequencing was performed by the DNA Services (DNAS) core of the RRC.

### 1. Sequence Merging.

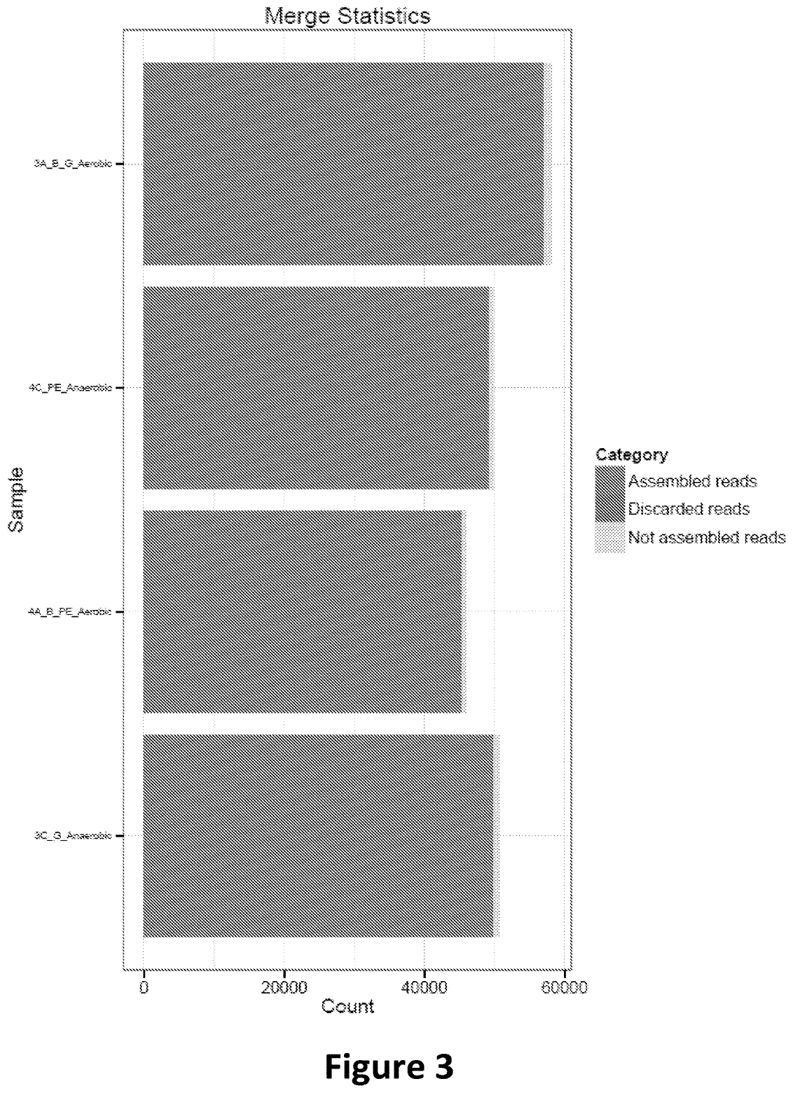

Forward and reverse reads were merged using PEAR (Jiajie Zhang, et al. Pear: a fast and accurate illumina paired-end read merger. Bioinformatics, 30(5):614-620, Mar. 12014). Percentages displayed are calculated based on the number of sequences per sample at the start of merging (Table 15 and FIG. 3).

TABLE 15

| Sample | Discarded reads | Unassembled reads | Assembled reads |
|---|---|---|---|
| 3A_B_G_Aerobic | 0 (0%) | 921 (1.59%) | 57177 (98.41%) |
| 4C_PE_Anaerobic | 0 (0%) | 697 (1.40%) | 49224 (98.60%) |
| 4A_B_PE_Aerobic | 0 (0%) | 701 (1.52%) | 45307 (98.48%) |
| 3C_G_Anaerobic | 0 (0%) | 712 (1.41%) | 49881 (98.59%) |

### 2. Trimming.

The DNAS core utilizes a set of filters and trim steps to generate a sequence dataset containing high quality data. Percentages displayed are calculated based on the number of sequences per sample at the start of trimming.

1. Ambiguous nucleotides (N) are trimmed from the ends and reads with internal ambiguous nucleotides are discarded.

2. Primer sequences are trimmed from the reads and any that lack either primer are discarded.

(SEQ ID NO: 1)
Forward primer 515F GTGCCAGCMGCCGCGGTAA
(SEQ ID NO: 2)
Reverse primer 926R CCGYCAATTYMTTTRAGTTT

3. Reads are trimmed using a quality threshold of p=0:01.

4. Reads, after trimming, that are less than 300 bp in length are discarded.

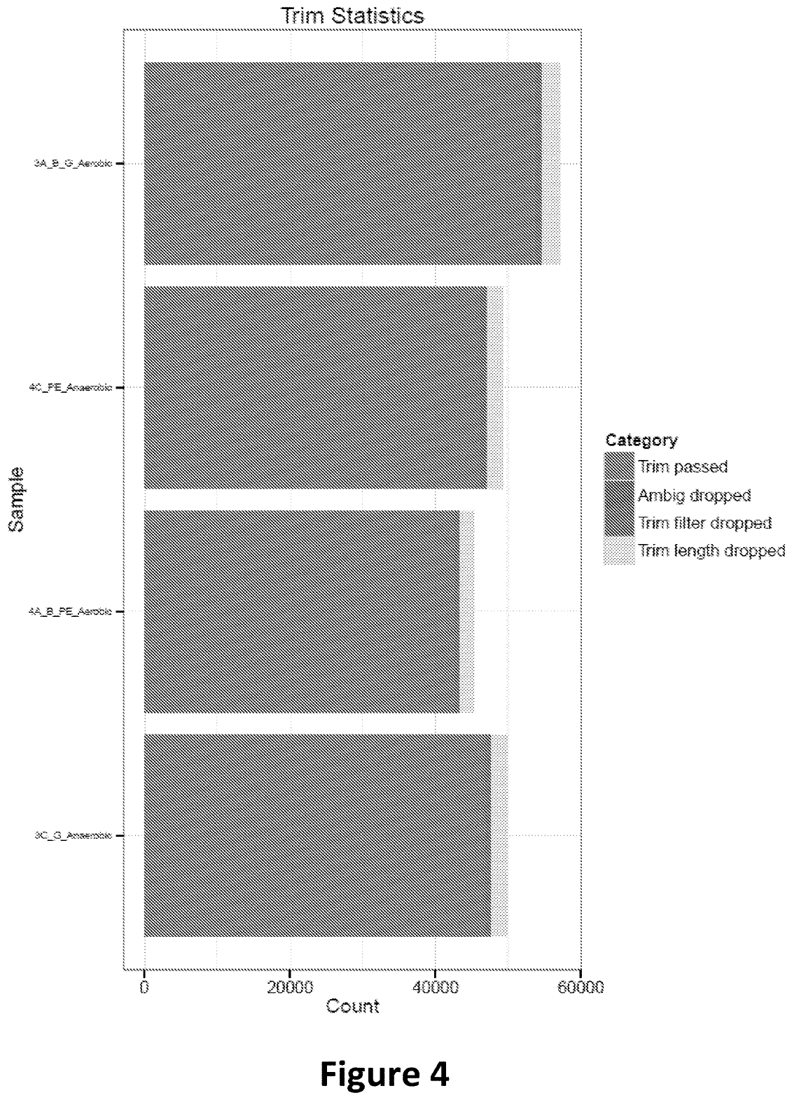

### 2.1. Basic Statistics (Table 16 and FIG. 4).

TABLE 16

| | Trimmed | Reads missing | Ambiguity | Short length | |
|---|---|---|---|---|---|
| Sample | on quality | primers | discards | discards | Reads passed |
| 3A_B_G_Aerobic | 2996 (5.24%) | 874 (1.53%) | 1 (0%) | 2516 (4.40%) | 53786 (94.07%) |
| 4C_PE_Anaerobic | 2554 (5.19%) | 762 (1.55%) | 0 (0%) | 2149 (4.37%) | 46313 (94.09%) |
| 4A_B_PE_Aerobic | 2389 (5.27%) | 654 (1.44%) | 1 (0%) | 1989 (4.39%) | 42663 (94.16%) |
| 3C_G_Anaerobic | 2592 (5.20%) | 868 (1.74%) | 2 (0%) | 2196 (4.40%) | 46815 (93.85%) |

### 2.2. Read Lengths (Table 17).

TABLE 17

| | | |
|---|---|---|
| | Mean length before | Mean length after |
| Sample | trimming | trimming |

| | | | | |
|---|---|---|---|---|
| 3A_B_G_Aerobic | 410.1 | bp | 372 | bp |
| 4C_PE_Anaerobic | 409.9 | bp | 371.8 | bp |
| 4A_B_PE_Aerobic | 410.6 | bp | 372.4 | bp |
| 3C_G_Anaerobic | 410 | bp | 371.9 | bp |

### 3. Chimera Filtering.

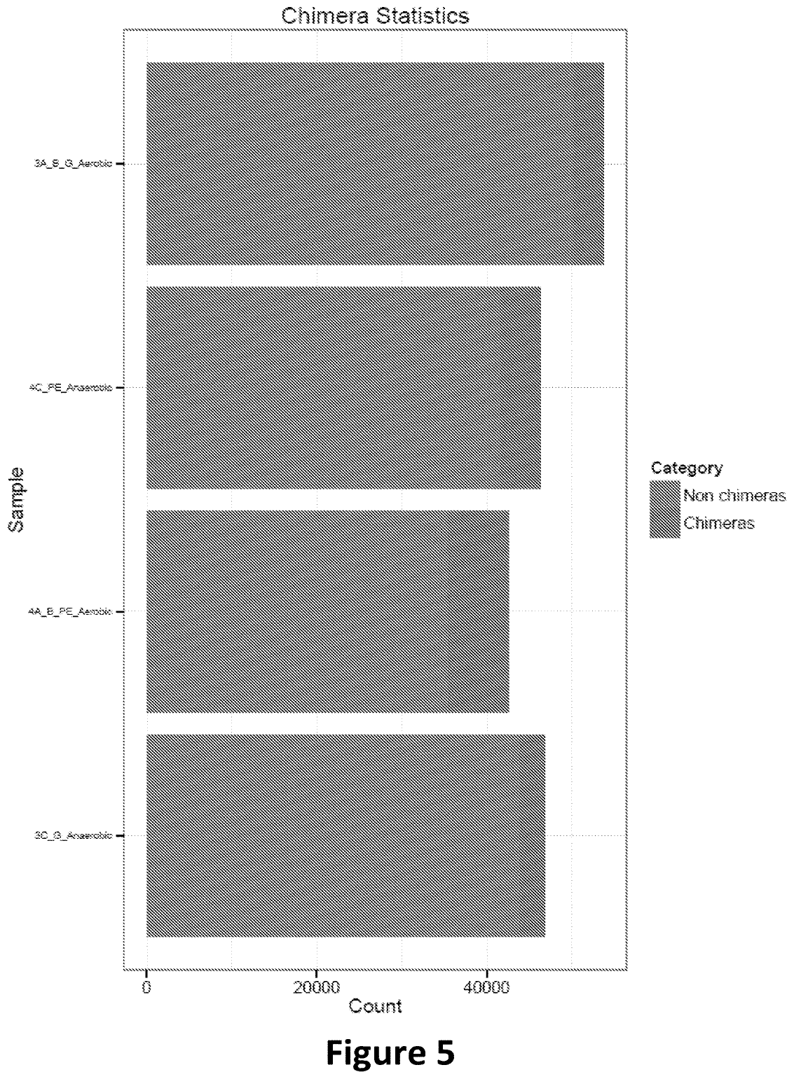

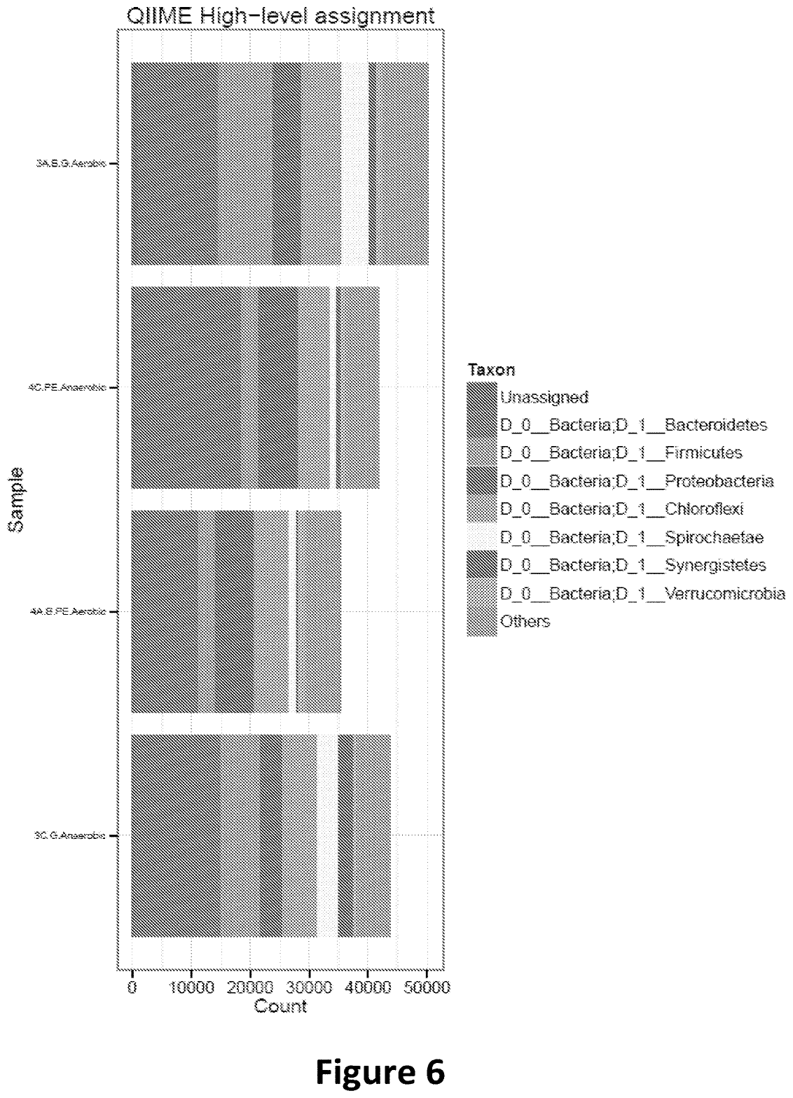

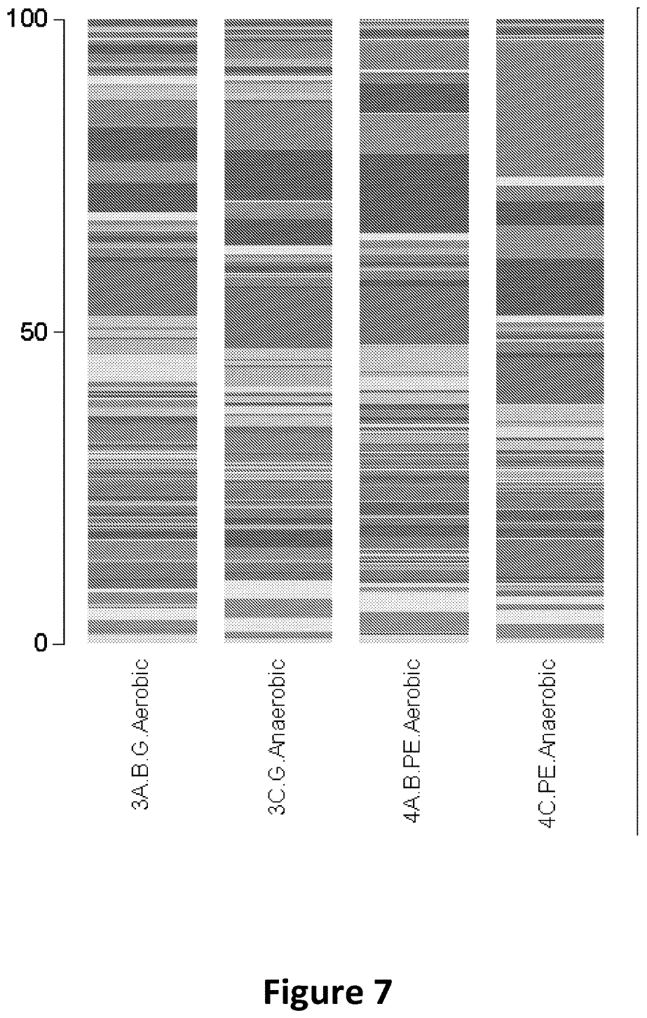

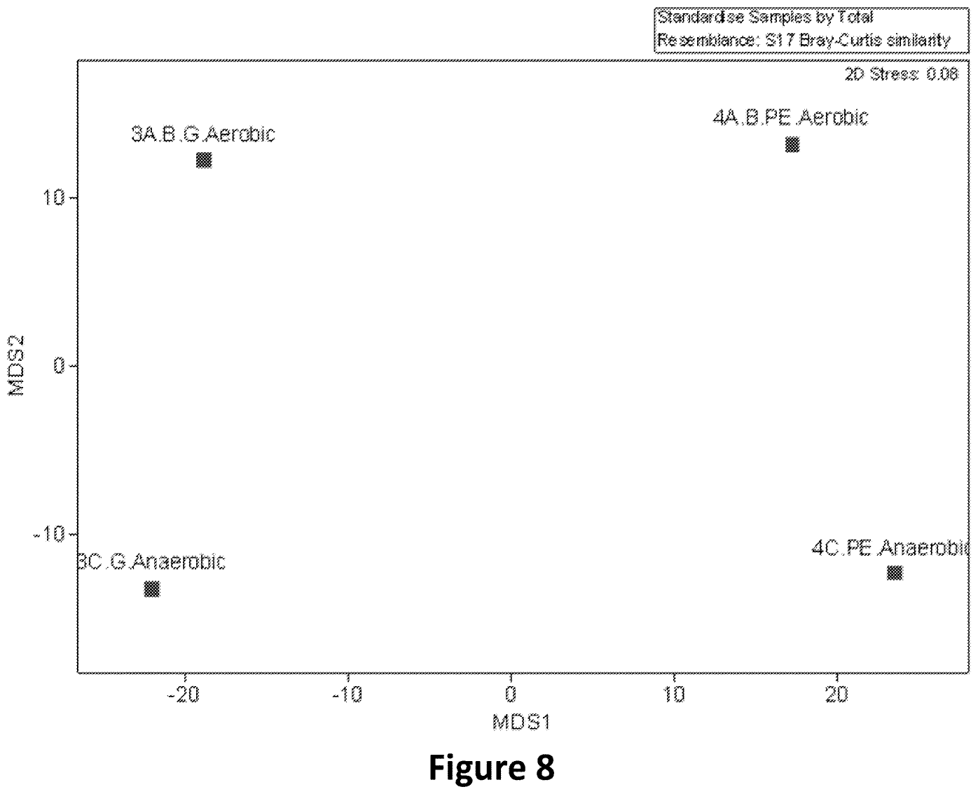

Chimeric sequences are artifacts of the PCR process and occur when portions of two separate amplicons fuse during the amplification process. The RRC analysis pipeline uses a standard chimera checking program to identify chimeric sequences and remove from the dataset. Briefly, chimeric sequences were identified using the UCHIME algorithm as compared with the Greengenes_13_8_97database (Robert C. Edgar. Search and clustering orders of magnitude faster than blast. Bioinformatics, 26(19):2460-2461, 2010). Percentages displayed are calculated based on the number of sequences per sample prior to chimera filtering (Table 18 and FIG. 5).

TABLE 18

| Sample | Chimeric sequences | Valid sequences (passed) |
|---|---|---|
| 3A_B_G_Aerobic | 3505 (6.52%) | 50281 (93.48%) |
| 4C_PE_Anaerobic | 4608 (9.95%) | 41705 (90.05%) |
| 4A_B_PE_Aerobic | 7274 (17.05%) | 35389 (82.95%) |
| 3C_G_Anaerobic | 2975 (6.35%) | 43840 (93.65%) |

### 4. QIIME Pipeline.

The QIIME v1.8 pipeline was used to perform the following processing steps: (1) Prepared sequence files (FASTA format) were combined using sample information; (2) Operational taxonomic unit (OTU) clusters were generated denovo manner using the UCLUST method; (3) Representative sequences for each OTU were generated; (4) Taxonomic annotations were assigned to each OTU using the representative sequence data using UCLUST and the silva 119 16S.97 reference database (Daniel McDonald, et al. An improved greengenes taxonomy with explicit ranks for ecological and evolutionary analyses of bacteria and archaea. The ISME Journal, 6(3):610-618, March 2012); (5) Taxonomic and OTU abundance data were merged into a single OTU table and summaries of absolute abundances of taxa were generated for all phyla, classes, orders, families, genera, and species present in the dataset. (J. G. Caporaso, et al. Qiime allows analysis of high-throughput community sequencing data. Nature methods, 7(5):335-336, May 2010).

### Example 4

Scaled-up test runs were performed with grass, dried leaves, phragmites, and cattails under anaerobic conditions at 35° C. and 120 RPM mixing conditions to verify if yield was affected by the scaling-up process. These scaled-up runs used about 60 to 80 times higher liquid volume than previous serum bottle experiments (e.g., serum bottle experiments used a total liquid volume of 100 mL whereas the upscale experiments conducted in a fermenter used a total liquid volume of 6,000 to 8,000 mL.

### Example 5

### Stock Culture Maintenance pH and Temperature

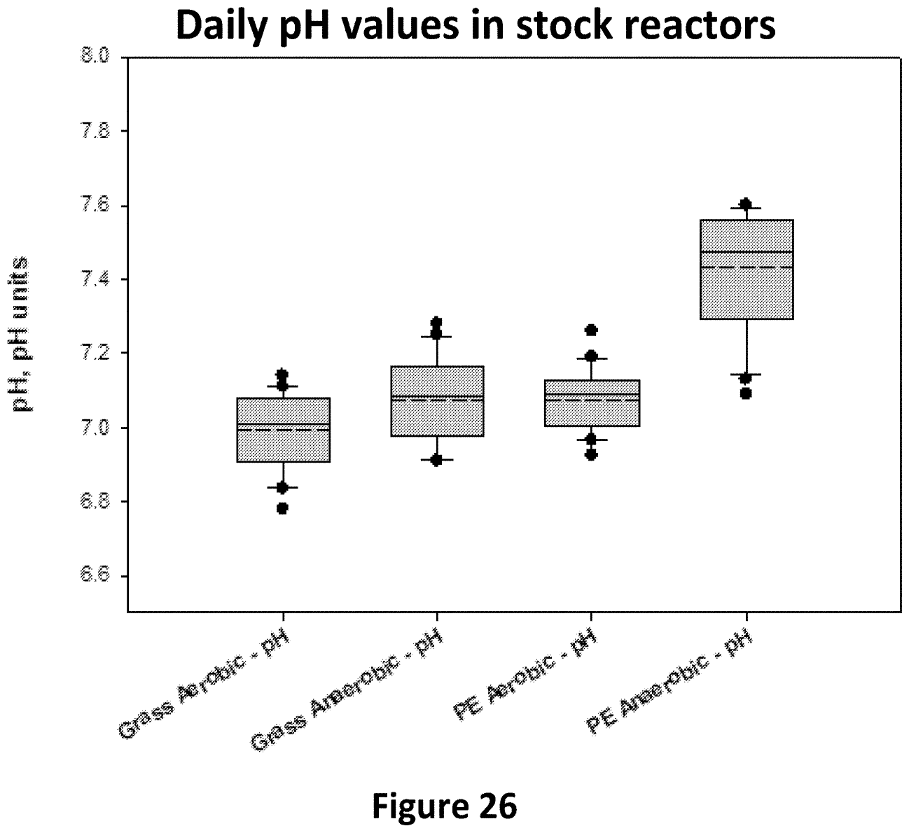

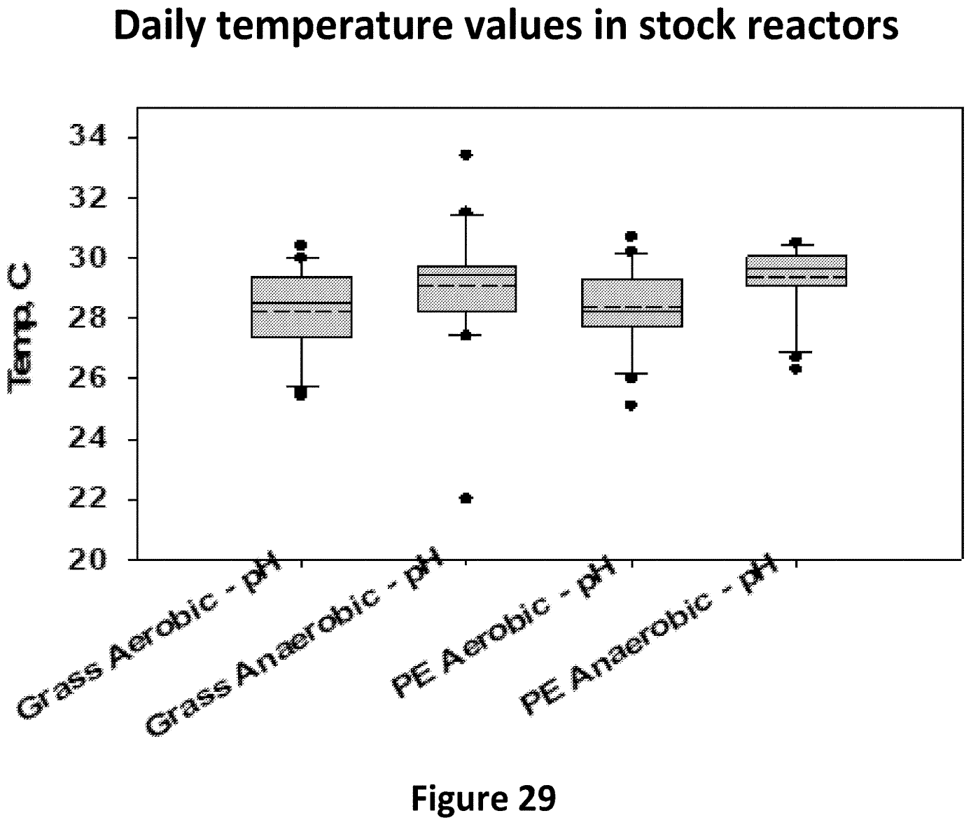

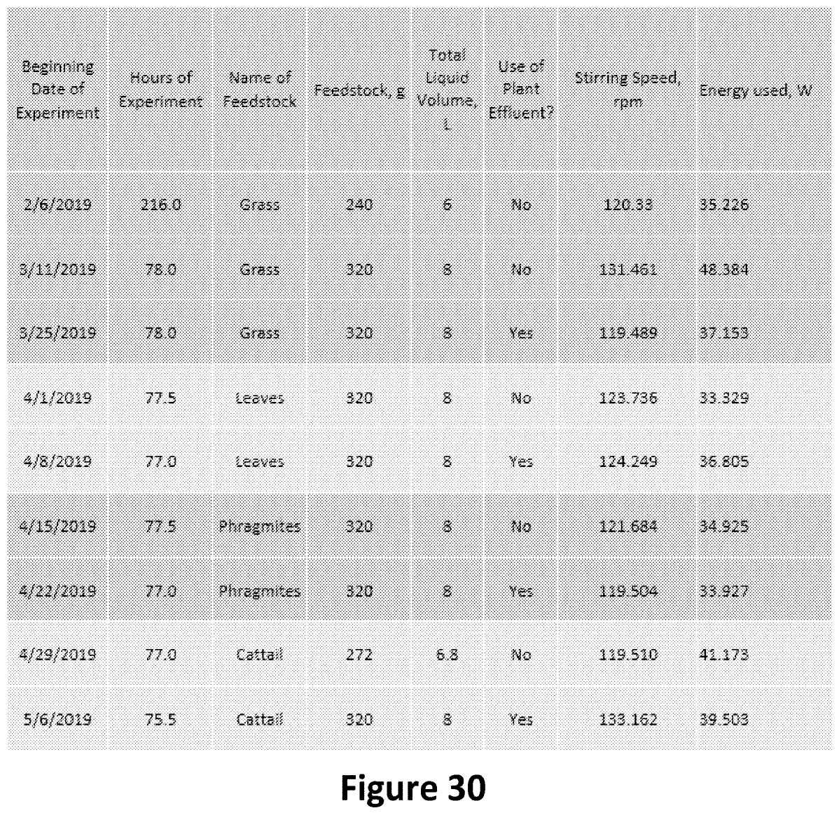

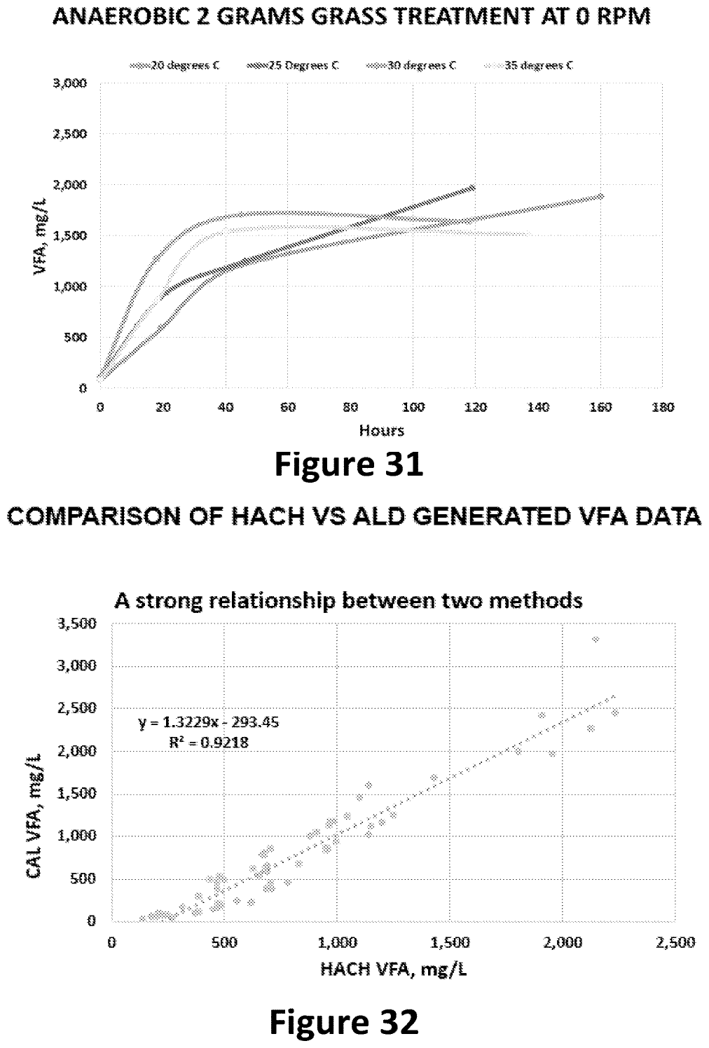

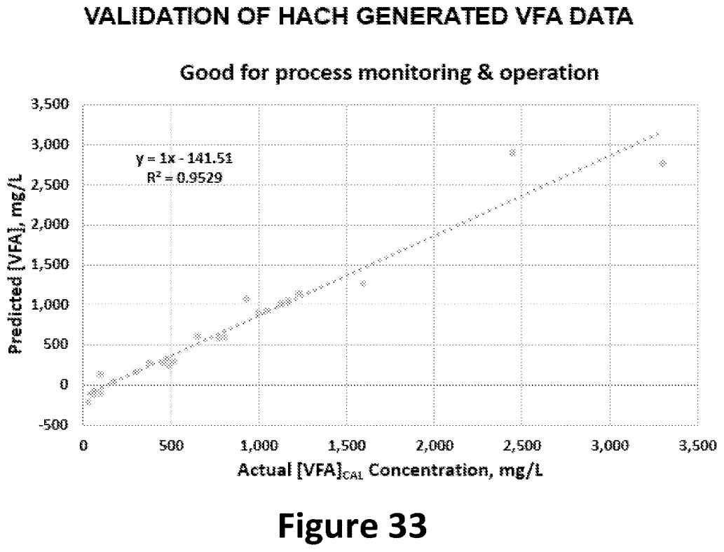

Stock culture pH values observed with the use of alkalinity buffer solution were slightly above 7.0, while for anaerobic guar gum reactor, just below 7.50 as shown FIG. 26 and Table 19. This allowed methanogens to reside in the system and contribute in gas production. Stock culture temperature values observed are shown in FIG. 29.

TABLE 19
pH and Temperature Characteristics of Stock Cultures

| | Grass Aerobic | | Grass Anaerobic | | Guar Gum Aerobic | | Guar Gum Anaerobic | |
|---|---|---|---|---|---|---|---|---|
| Statistic | pH | Temp | pH | Temp | pH | Temp | pH | Temp |
| Minimum | 6.78 | 25.40 | 6.91 | 22.00 | 6.93 | 25.10 | 7.09 | 26.30 |
| Maximum | 7.14 | 30.40 | 7.28 | 33.40 | 7.26 | 30.70 | 7.60 | 30.50 |
| Average | 6.99 | 28.23 | 7.07 | 29.08 | 7.07 | 28.36 | 7.43 | 29.37 |
| Median | 7.01 | 28.50 | 7.09 | 29.40 | 7.09 | 28.20 | 7.48 | 29.65 |
| Std Dev | 0.10 | 1.36 | 0.11 | 2.15 | 0.09 | 1.36 | 0.16 | 1.11 |

### Fermenter Scale pH

pH values observed at larger fermenter scale for different feedstocks under anaerobic condition at 35 C, 120 RPM are shown in Table 20A and Table 20B, which indicates suppressed pH values compared to smaller scale samples (noticeably for grass).

TABLE 20A

| | | | | Total | | Nutrient | Plant |
|---|---|---|---|---|---|---|---|
| Beginning | | | Feed- | Liquid | Inoculum | Solution | Effluent |
| Date of | Hours of | Name of | stock | Volume | Volume | Volume | Volume |
| Experiment | Experiment | Feedstock | (g) | (L) | (L) | (L) | (L) |
| Feb. 6, 2019 | 216 | Grass | 240 | 6 | 3 | 3 | |
| Mar. 11, 2019 | 78 | Grass | 320 | 8 | 4 | 4 | |
| Mar. 25, 2019 | 78 | Grass | 320 | 8 | 4 | | 4 |
| Apr. 1, 2019 | 77.5 | Leaves | 320 | 8 | 4 | 4 | |
| Apr. 8, 2019 | 77 | Leaves | 320 | 8 | 4 | | 4 |
| Apr. 15, 2019 | 77.5 | Phragmites | 320 | 8 | 4 | 4 | |
| Apr. 22, 2019 | 77 | Phragmites | 320 | 8 | 4 | | 4 |
| Apr. 29, 2019 | 77 | Cattails | 272 | 6.8 | 3.4 | 3.4 | |
| May 6, 2019 | 75.5 | Cattails | 320 | 8 | 4 | | 4 |

TABLE 20B

| Beginning | | | pH | pH | pH | pH | pH |
|---|---|---|---|---|---|---|---|
| Date of | Hours of | Name of | (Min) | (Max) | (Avg) | (Median) | (Std Dev) |
| Experiment | Experiment | Feedstock | pH units | pH units | pH units | pH units | pH units |
| Feb. 6, 2019 | 216 | Grass | 5.93 | 7.16 | 6.61 | 6.84 | 0.40 |
| Mar. 11, 2019 | 78 | Grass | n/a | n/a | n/a | n/a | n/a |
| Mar. 25, 2019 | 78 | Grass | 5.26 | 7.16 | 5.43 | 5.33 | 0.34 |
| Apr. 1, 2019 | 77.5 | Leaves | 6.13 | 7.08 | 6.43 | 6.32 | 0.25 |
| Apr. 8, 2019 | 77 | Leaves | 5.45 | 7.15 | 5.69 | 5.60 | 0.29 |
| Apr. 15, 2019 | 77.5 | Phragmites | 6.50 | 7.76 | 6.81 | 6.85 | 0.19 |
| Apr. 22, 2019 | 77 | Phragmites | 6.50 | 7.76 | 6.76 | 6.73 | 0.17 |
| Apr. 29, 2019 | 77 | Cattails | 5.90 | 7.97 | 6.78 | 6.81 | 0.50 |
| May 6, 2019 | 75.5 | Cattails | 5.90 | 7.94 | 6.57 | 6.24 | 0.50 |

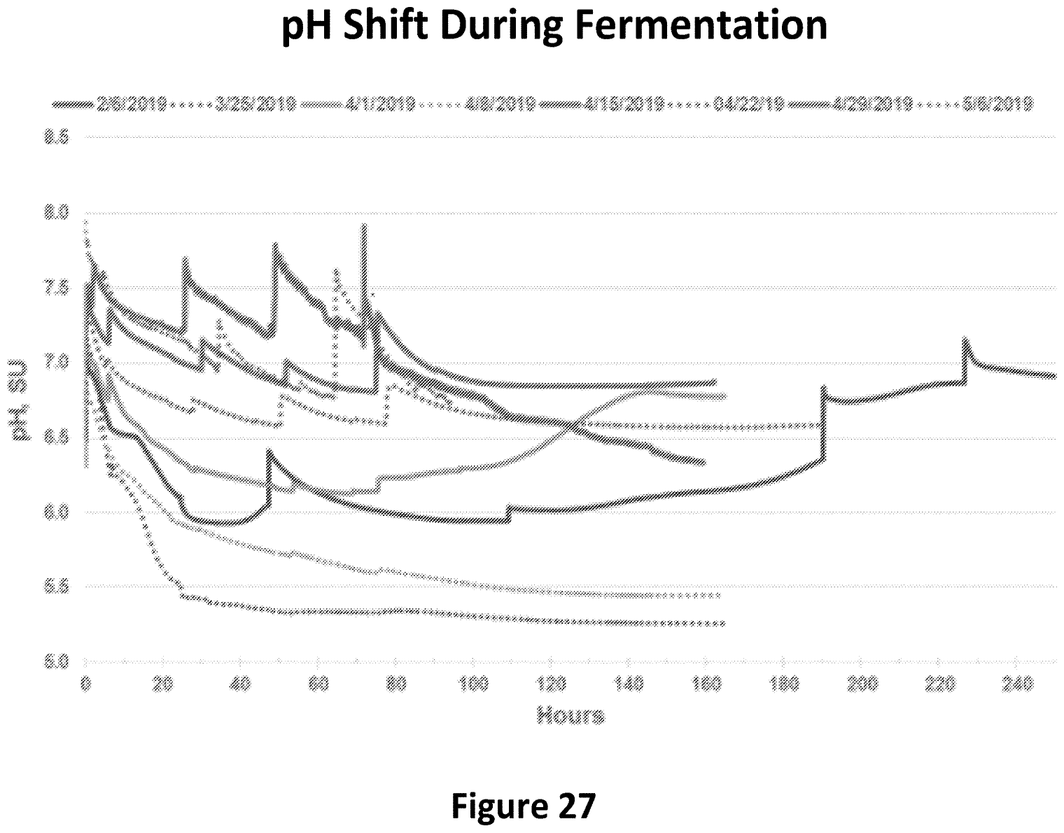

The online pH drop monitored during larger scale runs is shown in FIG. 27. Acceptable enzymatic activity of acidogens (acid formers) occurs within the pH range of 5.0 to 6.2 whereas of methanogens occurs within the pH range of 6.8 to 7.2.

### Example 6

A representative example of a cellulose feedstock specific cultivation procedure is described below for cultivating and populating cellulolytic organisms' culture. In this procedure, grass is used as a cellulose feedstock but grass may be replaced by any other desirable feedstock.

a. Collect a sufficient amount (2 L) of digester draw.

b. Prepare the reactor (either a 5-L fermenter or a 5-L container with airtight lid) consisting of 1,500 mL digester draw, 1,500 mL of secondary treatment effluent, 100 g of dried shredded grass in powder form (just to make the solubilization step of fermentation process easier and increasing surface ratio of substrate to bugs; also to prevent feed and draw tubing clogged) and 3 g cellulase (can be used in initial set-up stage though not necessarily required; its use can shorten cultivation time).

c. Close the lid, shake and mix the contents well and leave undisturbed to allow for the organisms within the reactor to mature for about one month. The reactor lid can contain a thin tubing pierced through the lid for the biogas to vent out as a safety measure. If the reactor is a fermenter, then the gas venting with one way check valve mechanism should be used.

d. After about at least one-month time period, open the container and decant stratified liquid to discard.

e. Recover sludge portion (almost no separable grass is expected at this stage), and transfer 1 L content to each of the two separate fermenters; one aerobic and another anaerobic (see below Aerobic Fermenter and Anaerobic Fermenter).

f. At a frequency of every third day, a draw of 500 mL of content from both types of reactors is taken and replenished with 500 mL of a nutrient solution and 22 g of grass (or at a rate of 2 g per liter of fermenter liquid volume).

g. In case of guar gum, substance addition will be 1.1 g, or at a rate of 0.1 g per liter of fermenter liquid volume.

h. The draw from every third day can be sampled and analyzed for total solids (TS), volatile solids (VS), chemical oxygen demand (COD), total phosphorus (TP), ortho-phosphates (Ortho-P), alkalinity, VFA, total kjeldahl nitrogen (TKN), and ammonia nitrogen (NH₃) whereas inside content can be monitored for temperature and pH. As long as temperature is within the intended range, it should work like a controlled system. Too high of TKN is broken down into NH₃, which in turn can potentially induce ammonia toxicity to the bugs. Relatively stable TS, VS, and COD values will indicate that the system is in steady state and is ready for the use.

i. Continue this feed/draw cycle for at least one to two months until the system has reached steady state. The culture is expected to be ready for the use. The total draw in each instance should not be more than 1.5 L per week to prevent a washout of community of organisms. Continue the routine like described above as long as you need the stock culture it for experimentation.

### Anaerobic Fermenter:

Place 1 L of old content into Fermenter without much exposure to atmosphere and mix with 5 L of digester draw and 5 L of secondary treated plant effluent. Total volume is 11 L. Remove air by flushing nitrogen gas and close the lid airtight. Intermittently mix the container by auto or manual operation at 120 RPM. Fermenter temperature is maintained between 30° C. and 35° C.

### Aerobic Fermenter:

Place 1 L of old content into Fermenter and mix with 5 L of digester draw and 5 L of secondary treated plant effluent. Total volume is 11 L. Shake well and mix thoroughly. Leave the lid half open for allowing air to exchange. Although there seemed to be no need for mechanical mixing and/or aeration; intermittent mixing and aeration may be helpful. Occasional shaking can be helpful. Maintain fermenter temperature between 30° C. and 35° C.

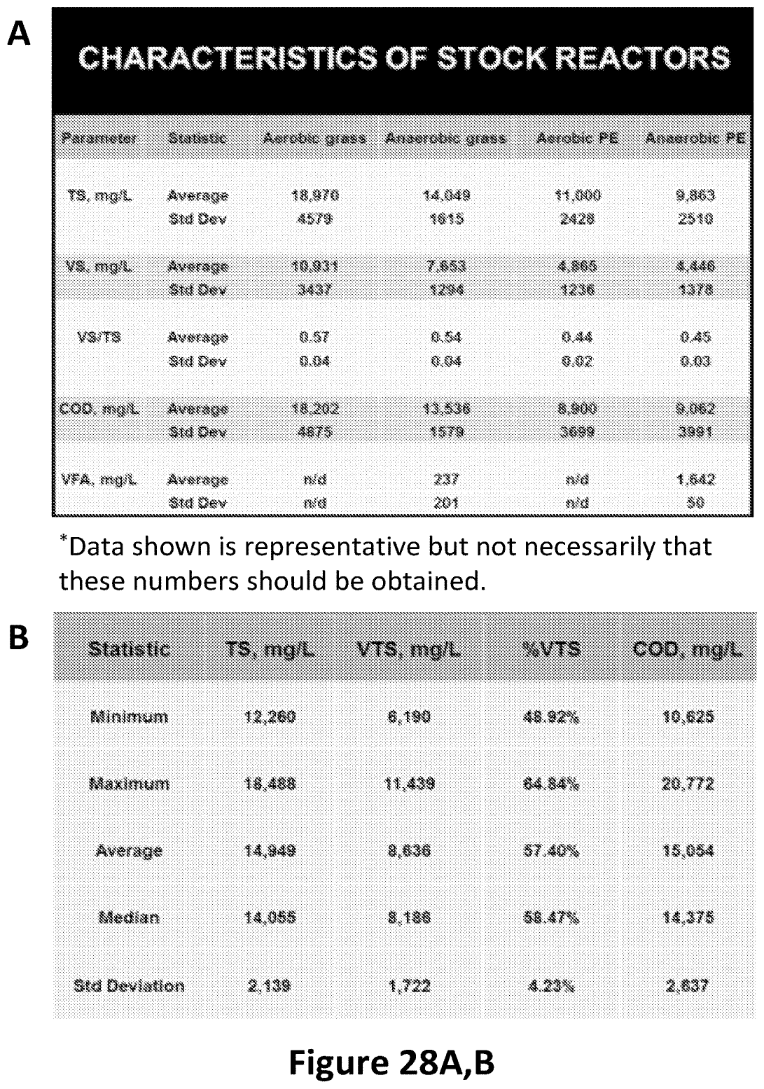

Representative data of characteristics of stock reactors are shown in FIG. 28 and FIG. 29.

### Example 7

### Additional Observations.

TABLE 21
Serum Bottle Test VFA Yield Test Results in Milligrams
of VFA per Dry Gram of Grass (Based on 100% dry basis).

| | | |
|---|---|---|
| Grass | Max anaerobic yield, (mg/g) | 202.1 |
| | Min anaerobic yield, (mg/g) | 74.8 |
| Grass | Max aerobic yield, (mg/g) | 106.8 |
| | Min aerobic yield, (mg/g) | 38.7 |

Average VFA yield for grass ranged from 38.7 to 106.8 mg per gram in aerobic and from 74.8 to 129.3 mg per gram in anaerobic condition with a maximum VFA yield of 202.1 mg/g in 4 g grass treatment (FIG. 29).

TABLE 22
Serum Bottle Test Total Gas Yield Test Results in Milligrams
of Gas per Dry Gram of Grass (Based on 100% dry basis).

| | Total | Total | Gas Yield (5.18% | Gas Yield (0% |
|---|---|---|---|---|
| | Gas | Time | moisture; 94.82% dry) | moisture; 100% dry) |
| | CC | Hours | CC/g | CC/g |
| Min | 156.00 | 45.00 | 39.00 | 41.13 |
| Max | 365.00 | 160.00 | 91.25 | 96.23 |
| Average | 252 | 112 | 63 | 66 |

TABLE 23
Scaled-up Grass Fermenter Scale Test VFA Yield and Gas Yield Test
Results in Milligrams of VFA per Dry Gram of Grass and Milliliters
of total gas per Dry Gram of Grass (Based on 100% dry basis).

| | | | | Total | | 100% Dry Basis | |
|---|---|---|---|---|---|---|---|
| Beginning | | | | Liquid | Plant | Max VFA | Gas Yield at |
| Date of | Hours of | Name of | Feedstock | Volume | Effluent | Yield, mg/g | Final Hour |
| Experiment | Experiment | Feedstock | (g) | (L) | Use? | (Hrs) | (mL/g) |
| Feb. 6, 2019 | 216 | Grass | 240 | 6 | No, | 166.2 | 110.8 |
| | | | | | Nutrient | (113.5) | |
| | | | | | Soln | | |
| Mar. 11, 2019 | 78 | Grass | 320 | 8 | No, | 150.9 | N/A |
| | | | | | Nutrient | (78.0) | |
| | | | | | Soln | | |
| Mar. 25, 2019 | 78 | Grass | 320 | 8 | Yes | 152.0 | 34.8 |
| | | | | | | (78.0) | |

TABLE 24
Scaled-up Leaves, Phragmites, and Cattails as Feedstock Fermenter Scale
Test VFA Yield and Gas Yield Test Results in Milligrams of VFA per Gram
of Feedstock* and Milliliters of total gas per Gram of Feedstock*.

| | | | | Total | | | |
|---|---|---|---|---|---|---|---|
| Beginning | | | | Liquid | Plant | Max VFA | Gas Yield at |
| Date of | Hours of | Name of | Feedstock | Volume | Effluent | Yield mg/g | Final Hour |
| Experiment | Experiment | Feedstock | (g) | (L) | Use?** | (Hrs) | (mL/g) |
| Apr. 1, 2019 | 77.5 | Leaves | 320 | 8 | No, | 98.6 | 30.6 |
| | | | | | Nutrient | (77.5) | |
| | | | | | Soln | | |
| Apr. 8, 2019 | 77 | Leaves | 320 | 8 | Yes | 102.3 | 24.2 |
| | | | | | | (77.0) | |
| Apr. 15, 2019 | 77.5 | Phragmites | 320 | 8 | No, | 16.4 | 28.9 |
| | | | | | Nutrient | (77.5) | |
| | | | | | Soln | | |
| Apr. 22, 2019 | 77 | Phragmites | 320 | 8 | Yes | 12.6 | 20.6 |
| | | | | | | (77.0) | |
| Apr. 29, 2019 | 77 | Cattail | 272 | 6.8 | No, | 26.5 | 14.7 |
| | | | | | Nutrient | (77.0) | |
| | | | | | Soln | | |
| May 6, 2019 | 75.5 | Cattail | 320 | 8 | Yes | 22.8 | 12.8 |
| | | | | | | (75.5) | |

*Moisture content undetermined and thus values not corrected for moisture content.
**These experiments used nutrient solution and same experiment without nutrient solution, replacing by plant effluent.

TABLE 25
GAS RANGE - ANAEROBIC mL/g

| | Wet basis | Dry basis | Duration hours |
|---|---|---|---|
| Phase I range | | | |
| Grass (1 to 6 g) | 34 to 179 | 36 to 189 | 189 |
| Phase I range | | | |
| Guar gum (0.5 to 4 g) | 248 to 408 | 261 to 430 | 168 |
| Phase II range | | | |
| Grass (4 g) | | 197 | 248.5 |
| Grass (6 g) | | 81 | 248.5 |
| Phase II range | | | |
| Leaves (1 g) | 165 | | 1082 |
| Leaves (2 g) | 202 | | 1082 |
| Leaves (3 g) | 190 | | 385.5 |
| Leaves (4 g) | 144 | | 1082 |
| Phragmites (1 g) | 323 | | 1082 |
| Phragmites (2 g) | 326 | | 1082 |
| Phragmites (3 g) | 303 | | 1082 |
| Phragmites (4 g) | 309 | | 1082 |
| Cattails (1 g) | 315 | | 1082 |
| Cattails (2 g) | 15 | | 26 |
| Cattails (3 g) | 219 | | 1082 |
| Cattails (4 g) | 181 | | 1082 |
| Grass (4 g) | 216 | 227 | 503 |
| Grass (4 g) | | | |
| Grass (4 g) | 45 | 48.0 | 166 |
| Leaves (4 g) | 80 | | 168 |
| Leaves (4 g) | 37 | | 166 |
| Phragmites (4 g) | 76 | | 167 |
| Phragmites (4 g) | 57 | | 166 |
| Cattails (4 g) | 16 | | 76 |
| Cattails (4 g) | 16 | | 75 |

TABLE 26A
Top 25 cellulolytic bacterial microorganisms under aerobic/grass fermentation.

| | |
|---|---|
| 1 | D_0_Bacteria; D_1_Chloroflexi; D_2_Anaerolineae; D_3_Anaerolineales; D_4_Anaerolineaceae; |
| | Other; Other Most abundant in all reactors |
| 2 | D_0_Bacteria; D_1_Bacteroidetes; D_2_SB-1; Other; Other; Other; Other |
| 3 | D_0_Bacteria; D_1_Bacteroidetes; D_2_vadinHA17; Other; Other; Other; Other |
| 4 | D_0_Bacteria; D_1_Firmicutes; D_2_Clostridia; D_3_Clostridiales; D_4_Family |
| | XI; D_5_Sedimentibacter; Other |
| 5 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 6 | D_0_Bacteria; D_1_Bacteroidetes; D_2_Sphingobacteriia; D_3_Sphingobacteriales; D_4_WCHB1-69; |
| | Other; Other |
| 7 | D_0_Bacteria; D_1_Spirochaetae; D_2_Spirochaetes; D_3_Spirochaetes Incertae |
| | Sedis; D_4_Unknown Family; D_5_Candidatus Cloacamonas; Other |
| 8 | D_0_Bacteria; D_1_Firmicutes; D_2_Clostridia; D_3_Clostridiales; D_4_Ruminococcaceae; |
| | Other; Other |
| 9 | D_0_Bacteria; D_1_Spirochaetae; D_2_Spirochaetes; D_3_Spirochaetales; D_4_Spirochaetaceae; |
| | D_5_Treponema; Other |
| 10 | D_0_Bacteria; D_1_Chloroflexi; Other; Other; Other; Other; Other |
| 11 | D_0_Bacteria; D_1_Bacteroidetes; D_2_Bacteroidia; D_3_Bacteroidales; D_4_Porphyromonadaceae; |
| | Other; Other |
| 12 | D_0_Bacteria; D_1_Synergistetes; D_2_Synergistia; D_3_Synergistales; D_4_Synergistaceae; |
| | Other; Other |
| 13 | D_0_Bacteria; D_1_Thermotogae; D_2_Thermotogae; D_3_Thermotogales; D_4_Thermotogaceae; |
| | D_5_SC103; Other |
| 14 | D_0_Bacteria; D_1_Firmicutes; D_2_Clostridia; D_3_Clostridiales; D_4_Christensenellaceae; |
| | Other; Other |
| 15 | Unassigned; Other; Other; Other; Other; Other; Other |
| 16 | D_0_Bacteria; D_1_Bacteroidetes; D_2_Bacteroidetes Incertae Sedis; D_3_Unknown |
| | Order; D_4_Unknown Family; D_5_Prolixibacter; Other |
| 17 | D_0_Bacteria; D_1_Chloroflexi; D_2_Anaerolineae; D_3_Anaerolineales; D_4_Anaerolineaceae; |
| | D_5_Leptolinea; Other |
| 18 | D_0_Bacteria; D_1_Verrucomicrobia; D_2_OPB35 soil group; Other; Other; Other; Other |
| 19 | D_0_Bacteria; D_1_Firmicutes; D_2_Clostridia; D_3_Clostridiales; D_4_Ruminococcaceae; |
| | D_5_Incertae Sedis; Other |
| 20 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 21 | D_0_Bacteria; D_1_Proteobacteria; D_2_Deltaproteobacteria; D_3_Deltaproteobacteria |
| | Incertae Sedis; D_4_Syntrophorhabdaceae; D_5_Syntrophorhabdus; Other |
| 22 | D_0_Bacteria; D_1_Candidate division BRC1; Other; Other; Other; Other; Other |
| 23 | D_0_Bacteria; D_1_Spirochaetae; D_2_Spirochaetes; Other; Other; Other; Other |
| 24 | D_0_Bacteria; D_1_Bacteroidetes; D_2_Bacteroidia; D_3_Bacteroidales; D_4_Porphyromonadaceae; |
| | D_5_Paludibacter; Other |
| 25 | D_0_Bacteria; D_1_Firmicutes; D_2_Clostridia; D_3_Clostridiales; D_4_Ruminococcaceae; |
| | D_5_Fastidiosipila; Other |

TABLE 26B
Top 10 cellulolytic bacterial microorganisms under aerobic/grass fermentation.

| | |
|---|---|
| 1 | D_0_Bacteria; D_1_Chloroflexi; D_2_Anaerolineae; D_3_Anaerolineales; D_4_Anaerolineaceae; |
| | Other; Other Most abundant in all reactors |
| 2 | D_0_Bacteria; D_1_Bacteroidetes; D_2_SB-1; Other; Other; Other; Other |
| 3 | D_0_Bacteria; D_1_Bacteroidetes; D_2_vadinHA17; Other; Other; Other; Other |
| 4 | D_0_Bacteria; D_1_Firmicutes; D_2_Clostridia; D_3_Clostridiales; D_4_Family |
| | XI; D_5_Sedimentibacter; Other |
| 5 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 6 | D_0_Bacteria; D_1_Bacteroidetes; D_2_Sphingobacteriia; D_3_Sphingobacteriales; |
| | D_4_WCHB1-69; Other; Other |
| 7 | D_0_Bacteria; D_1_Spirochaetae; D_2_Spirochaetes; D_3_Spirochaetes Incertae |
| | Sedis; D_4_Unknown Family; D_5_Candidatus Cloacamonas; Other |
| 8 | D_0_Bacteria; D_1_Firmicutes; D_2_Clostridia; D_3_Clostridiales; D_4_Ruminococcaceae; |
| | Other; Other |
| 9 | D_0_Bacteria; D_1_Spirochaetae; D_2_Spirochaetes; D_3_Spirochaetales; D_4_Spirochaetaceae; |
| | D_5_Treponema; Other |
| 10 | D_0_Bacteria; D_1_Chloroflexi; Other; Other; Other; Other; Other |

TABLE 27A
Top 25 cellulolytic microorganisms under aerobic/grass fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae sedis; |
| | p_Firmicutes; c_Bacilli; o_Bacillales; f_Planococcaceae; g_Sporosarcina; s_Sporosarcina |
| | psychrophila |
| 3 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 4 | sk_Eukaryota; k_Metazoa; p_Chordata; c_Mammalia; o_Primates; f_Hominidae; g_Homo; |
| | s_Homo sapiens |
| 5 | sk_Bacteria; k_Bacteria incertae sedis; |
| | p_Actinobacteria; c_Actinobacteria; Other; Other; Other; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Mammalia incertae sedis; |
| | f_Bovidae; g_Ovis; s_Ovis canadensis |
| 8 | sk_Eukaryota; k_Metazoa; p_Chordata; c_Actinopteri; o_Cypriniformes; f_Cyprinidae; |
| | g_Cyprinus; s_Cyprinus carpio |
| 9 | sk_Eukaryota; k_Metazoa; p_Chordata; c_Actinopteri; o_Beloniformes; f_Adrianichthyidae; |
| | g_Oryzias; s_Oryzias latipes |
| 10 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 11 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 12 | sk_Eukaryota; k_Metazoa; p_Chordata; c_Actinopteri; o_Cypriniformes; f_Cyprinidae; |
| | g_Danio; s_Danio rerio |
| 13 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 14 | sk_Eukaryota; k_Metazoa; p_Chordata; c_Aves; o_Apterygiformes; f_Apterygidae; |
| | g_Apteryx; s_Apteryx australis |
| 15 | sk_Bacteria; k_Bacteria incertae |
| | sedis; p_Proteobacteria; c_Gammaproteobacteria; o_Pseudomonadales; |
| | f_Pseudomonadaceae; g_Pseudomonas; s_Pseudomonas stutzeri |
| 16 | sk_Bacteria; k_Bacteria incertae |
| | sedis; p_Actinobacteria; c_Actinobacteria; o_Corynebacteriales; f_Mycobacteriaceae; |
| | g_Mycobacterium; s_Mycobacterium terrae |
| 17 | sk_Eukaryota; k_Viridiplantae; p_Streptophyta; c_Streptophyta incertae |
| | sedis; o_Solanales; f_Solanaceae; g_Solanum; s_Solanum lycopersicum |
| 18 | sk_Bacteria; k_Bacteria incertae |
| | sedis; p_Proteobacteria; c_Gammaproteobacteria; Other; Other; Other; Other |
| 19 | sk_Bacteria; k_Bacteria incertae |
| | sedis; p_Proteobacteria; c_Alphaproteobacteria; Other; Other; Other; Other |
| 20 | sk_Eukaryota; k_Viridiplantae; p_Streptophyta; c_Streptophyta incertae |
| | sedis; o_Vitales; f_Vitaceae; g_Vitis; s_Vitis vinifera |
| 21 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |
| 22 | sk_Eukaryota; k_Viridiplantae; p_Streptophyta; c_Liliopsida; o_Poales; f_Poaceae; g_Oryza; |
| | s_Oryza sativa |
| 23 | sk_Bacteria; k_Bacteria incertae |
| | sedis; p_Proteobacteria; c_Alphaproteobacteria; o_Rhizobiales; f_Bradyrhizobiaceae; |
| | g_Nitrobacter; s_Nitrobacter hamburgensis |
| 24 | sk_Bacteria; k_Bacteria incertae |
| | sedis; p_Firmicutes; c_Bacilli; o_Bacillales; f_Bacillaceae;g_Bacillus; Other |
| 25 | sk_Bacteria; k_Bacteria incertae |
| | sedis; p_Proteobacteria; c_Alphaproteobacteria; o_Rhizobiales; f_Bradyrhizobiaceae; |
| | g_Rhodopseudomonas; s_Rhodopseudomonas palustris |

TABLE 27B
Top 15 cellulolytic microorganisms under aerobic/grass fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Firmicutes; c_Bacilli; o_Bacillales; f_Planococcaceae; g_Sporosarcina; s_Sporosarcina |
| | psychrophila |
| 3 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 4 | sk_Eukaryota; k_Metazoa; p_Chordata; c_Mammalia; o_Primates; f_Hominidae; g_Homo; |
| | s_Homo sapiens |
| 5 | sk_Bacteria; k_Bacteria incertae |
| | sedis; p_Actinobacteria; c_Actinobacteria; Other; Other; Other; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Mammalia incertae |
| | sedis; f_Bovidae; g_Ovis; s_Ovis canadensis |
| 8 | sk_Eukaryota; k_Metazoa; p_Chordata; c_Actinopteri; o_Cypriniformes; f_Cyprinidae; |
| | g_Cyprinus; s_Cyprinus carpio |
| 9 | sk_Eukaryota; k_Metazoa; p_Chordata; c_Actinopteri; o_Beloniformes; f_Adrianichthyidae; |
| | g_Oryzias; s_Oryzias latipes |
| 10 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 11 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 12 | sk_Eukaryota; k_Metazoa; p_Chordata; c_Actinopteri; o_Cypriniformes; f_Cyprinidae; |
| | g_Danio; s_Danio rerio |
| 13 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 14 | sk_Eukaryota; k_Metazoa; p_Chordata; c_Aves; o_Apterygiformes; f_Apterygidae; |
| | g_Apteryx; s_Apteryx australis |
| 15 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; s_Pseudomonas stutzeri |

TABLE 27C
Top 10 cellulolytic microorganisms under aerobic/grass fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Firmicutes; c_Bacilli; o_Bacillales; f_Planococcaceae; g_Sporosarcina; s_Sporosarcina |
| | psychrophila |
| 3 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 4 | sk_Eukaryota; k_Metazoa; p_Chordata; c_Mammalia; o_Primates; f_Hominidae; g_Homo; |
| | s_Homo sapiens |
| 5 | sk_Bacteria; k_Bacteria incertae |
| | sedis; p_Actinobacteria; c_Actinobacteria; Other; Other; Other; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Mammalia incertae |
| | sedis; f_Bovidae; g_Ovis; s_Ovis canadensis |
| 8 | sk_Eukaryota; k_Metazoa; p_Chordata; c_Actinopteri; o_Cypriniformes; f_Cyprinidae; |
| | g_Cyprinus; s_Cyprinus carpio |
| 9 | sk_Eukaryota; k_Metazoa; p_Chordata; c_Actinopteri; o_Beloniformes; f_Adrianichthyidae; |
| | g_Oryzias; s_Oryzias latipes |
| 10 | sk_Bacteria; k_Bacteria incertae |
| | sedis; p_Actinobacteria; c_Actinobacteria; o_Streptomycetales; f_Streptomycetaceae; |
| | g_Streptomyces; Other |

TABLE 28A
Top 25 cellulolytic bacterial microorganisms under anaerobic/grass fermentation.

| | |
|---|---|
| 1 | D_0_Bacteria; D_1_Chloroflexi; D_2_Anaerolineae; D_3_Anaerolineales; D_4_Anaerolineaceae; |
| | Other; Other Most abundant in all reactors |
| 2 | D_0_Bacteria; D_1_Bacteroidetes; D_2_SB-1; Other; Other; Other; Other |
| 3 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 4 | D_0_Bacteria; D_1_Bacteroidetes; D_2_vadinHA17; Other; Other; Other; Other |
| 5 | D_0_Bacteria; D_1_Synergistetes; D_2_Synergistia; D_3_Synergistales; D_4_Synergistaceae; |
| | D_5_Thermovirga; Other |
| 6 | D_0_Bacteria; D_1_Firmicutes; D_2_Clostridia; D_3_Clostridiales; D_4_Christensenellaceae; |
| | Other; Other |
| 7 | D_0_Bacteria; D_1_Synergistetes; D_2_Synergistia; D_3_Synergistales; D_4_Synergistaceae; |
| | Other; Other |
| 8 | D_0_Bacteria; D_1_Firmicutes; D_2_Clostridia; D_3_Clostridiales; D_4_Ruminococcaceae; |
| | Other; Other |
| 9 | D_0_Bacteria; D_1_Spirochaetae; D_2_Spirochaetes; D_3_LNR A2-18; Other; Other; Other |
| 10 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 11 | D_0_Bacteria; D_1_Bacteroidetes; D_2_Sphingobacteriia; D_3_Sphingobacteriales; D_4_WCHB1-69; |
| | Other; Other |
| 12 | D_0_Bacteria; D_1_Thermotogae; D_2_Thermotogae; D_3_Thermotogales; D_4_Thermotogaceae; |
| | D_5_SC103; Other |
| 13 | D_0_Bacteria; D_1_Proteobacteria; D_2_Deltaproteobacteria; D_3_Deltaproteobacteria |
| | Incertae Sedis; D_4_Syntrophorhabdaceae; D_5_Syntrophorhabdus; Other |
| 14 | D_0_Bacteria; D_1_Chloroflexi; Other; Other; Other; Other; Other |
| 15 | D_0_Bacteria; D_1_Firmicutes; D_2_Clostridia; D_3_Clostridiales; D_4_Ruminococcaceae; |
| | D_5_Incertae Sedis; Other |
| 16 | D_0_Bacteria; D_1_Bacteroidetes; D_2_Bacteroidia; D_3_Bacteroidales; D_4_Porphyromonadaceae; |
| | D_5_Proteiniphilum; Other |
| 17 | D_0_Bacteria; D_1_Proteobacteria; D_2_Deltaproteobacteria; D_3_Syntrophobacterales; |
| | D_4_Syntrophaceae; D_5_Smithella; Other |
| 18 | D_0_Bacteria; D_1_Spirochaetae; D_2_Spirochaetes; D_3_Spirochaetales; D_4_Spirochaetaceae; |
| | D_5_Spirochaeta; Other |
| 19 | D_0_Bacteria; D_1_Chloroflexi; D_2_Anaerolineae; D_3_Anaerolineales; D_4_Anaerolineaceae; |
| | D_5_Leptolinea; Other |
| 20 | D_0_Bacteria; D_1_Candidate division BRC1; Other; Other; Other; Other; Other |
| 21 | D_0_Bacteria; D_1_Spirochaetae; D_2_Spirochaetes; D_3_Spirochaetes Incertae |
| | Sedis; D_4_Unknown Family; D_5_Candidatus Cloacamonas; Other |
| 22 | D_0_Bacteria; D_1_Bacteroidetes; D_2_Bacteroidia; D_3_Bacteroidales; D_4_M2PB4-65 |
| | termite group; Other; Other |
| 23 | D_0_Bacteria; D_1_Firmicutes; D_2_Clostridia; D_3_Ciostridiales; D_4_Lachnospiraceae; |
| | D_5_Incertae Sedis; Other |
| 24 | D_0_Bacteria; D_1_Firmicutes; D_2_Clostridia; D_3_Clostridiales; D_4_Ruminococcaceae; |
| | D_5_Saccharofermentans; Other |
| 25 | D_0_Bacteria; D_1_Bacteroidetes; D_2_Bacteroidia; D_3_Bacteroidales; D_4_Porphyromonadaceae; |
| | Other; Other |

TABLE 28B
Top 25 cellulolytic bacterial microorganisms under anaerobic/grass fermentation.

| | |
|---|---|
| 1 | D_0_Bacteria; D_1_Chloroflexi; D_2_Anaerolineae; D_3_Anaerolineales; D_4_Anaerolineaceae; |
| | Other; Other Most abundant in all reactors |
| 2 | D_0_Bacteria; D_1_Bacteroidetes; D_2_SB-1; Other; Other; Other; Other |
| 3 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 4 | D_0_Bacteria; D_1_Bacteroidetes; D_2_vadinHA17; Other; Other; Other; Other |
| 5 | D_0_Bacteria; D_1_Synergistetes; D_2_Synergistia; D_3_Synergistales; D_4_Synergistaceae; |
| | D_5_Thermovirga; Other |
| 6 | D_0_Bacteria; D_1_Firmicutes; D_2_Clostridia; D_3_Clostridiales; D_4_Christensenellaceae; |
| | Other; Other |
| 7 | D_0_Bacteria; D_1_Synergistetes; D_2_Synergistia; D_3_Synergistales; D_4_Synergistaceae; |
| | Other; Other |
| 8 | D_0_Bacteria; D_1_Firmicutes; D_2_Clostridia; D_3_Clostridiales; D_4_Ruminococcaceae; |
| | Other; Other |
| 9 | D_0_Bacteria; D_1_Spirochaetae; D_2_Spirochaetes; D_3_LNR A2-18; Other; Other; Other |
| 10 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |

TABLE 29A
Top 25 cellulolytic microorganisms under anaerobic/grass fermentation.

| | |
|---|---|
| 1 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae sedis; p_Proteobacteria; Other; Other; Other; Other; Other |
| 4 | sk_Bacteria; k_Bacteria incertae sedis; p_Candidatus Cloacimonetes; c_Candidatus |
| | Cloacimonetes incertae sedis; o_Candidatus Cloacimonetes incertae sedis; f_Candidatus |
| | Cloacimonetes incertae sedis; g_Candidatus Cloacimonas; s_Candidatus Cloacimonas |
| | acidaminovorans |
| 5 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 6 | sk_Eukaryota; k_Metazoa; p_Chordata; c_Mammalia; o_Primates; f_Hominidae; g_Homo; |
| | s_Homo sapiens |
| 7 | sk_Eukaryota; k_Metazoa; p_Chordata; c_Mammalia; o_Rodentia; f_Muridae; g_Mus; |
| | s_Mus musculus |
| 8 | sk_Eukaryota; k_Metazoa; p_Chordata; c_Mammalia; o_Mammalia incertae |
| | sedis; f_Bovidae; g_Ovis; s_Ovis canadensis |
| 9 | sk_Eukaryota; k_Metazoa; p_Chordata; c_Actinopteri; o_Cypriniformes; f_Cyprinidae; |
| | g_Cyprinus; s_Cyprinus carpio |
| 10 | sk_Eukaryota; k_Metazoa; p_Chordata; c_Actinopteri; o_Beloniformes; f_Adrianichthyidae; |
| | g_Oryzias; s Oryzias latipes |
| 11 | sk_Eukaryota; k_Metazoa; p_Chordata; c_Actinopteri; o_Cypriniformes; f_Cyprinidae; |
| | g_Danio; s_Danio rerio |
| 12 | sk_Bacteria; k_Bacteria incertae |
| | sedis; p_Actinobacteria; c_Actinobacteria; o_Propionibacteriales; f_Propionibacteriaceae; |
| | g_Tessaracoccus; s_Tessaracoccus sp. T2.5-30 |
| 13 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 14 | sk_Bacteria; k_Bacteria incertae |
| | sedis; p_Actinobacteria; c_Actinobacteria; o_Micrococcales; f_Intrasporangiaceae; |
| | g_Phycicoccus; s_Phycicoccus dokdonensis |
| 15 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |
| 16 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 17 | sk_Bacteria; k_Bacteria incertae |
| | sedis; p_Proteobacteria; c_Alphaproteobacteria; Other; Other; Other; Other |
| 18 | sk_Eukaryota; k_Metazoa; p_Chordata; c_Aves; o_Apterygiformes; f_Apterygidae; |
| | g_Apteryx; s_Apteryx australis |
| 19 | sk_Eukaryota; k_Viridiplantae; p_Streptophyta; c_Streptophyta incertae |
| | sedis; o_Solanales; f_Solanaceae; g_Solanum; s_Solanum lycopersicum |
| 20 | sk_Bacteria; k_Bacteria incertae |
| | sedis; p_Proteobacteria; c_Betaproteobacteria; o_Burkholderiales; Other; Other; Other |
| 21 | sk_Bacteria; k_Bacteria incertae |
| | sedis; p_Chloroflexi; c_Anaerolineae; o_Anaerolineales; f_Anaerolineaceae; g_Brevefilum; |
| | s_Brevefilum fermentans |
| 22 | sk_Eukaryota; k_Viridiplantae; p_Streptophyta; c_Streptophyta incertae |
| | sedis; o_Vitales; f_Vitaceae; g_Vitis; s_Vitis vinifera |
| 23 | sk_Bacteria; k_Bacteria incertae |
| | sedis; p_Actinobacteria; c_Actinobacteria; o_Propionibacteriales; f_Propionibacteriaceae; |
| | g_Tessaracoccus; s_Tessaracoccus flavescens |
| 24 | sk_Bacteria; k_Bacteria incertae |
| | sedis; p_Actinobacteria; c_Actinobacteria; o_Micrococcales; f_Intrasporangiaceae; |
| | g_Janibacter; s_Janibacter indicus |
| 25 | sk_Eukaryota; k_Viridiplantae; p_Streptophyta; c_Liliopsida; o_Poales; f_Poaceae; g_Oryza; |
| | s_Oryza sativa |

TABLE 29B
Top 15 cellulolytic microorganisms under anaerobic/grass fermentation.

| | |
|---|---|
| 1 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae sedis; p_Proteobacteria; Other; Other; Other; Other; Other |
| 4 | sk_Bacteria; k_Bacteria incertae sedis; p_Candidatus Cloacimonetes; c_Candidatus |
| | Cloacimonetes incertae sedis; o_Candidatus Cloacimonetes incertae sedis; f_Candidatus |
| | Cloacimonetes incertae sedis; g_Candidatus Cloacimonas; s_Candidatus Cloacimonas |
| | acidaminovorans |
| 5 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 6 | sk_Eukaryota; k_Metazoa; p_Chordata; c_Mammalia; o_Primates; f_Hominidae; g_Homo; |
| | s_Homo sapiens |
| 7 | sk_Eukaryota; k_Metazoa; p_Chordata; c_Mammalia; o_Rodentia; f_Muridae; g_Mus; |
| | s_Mus musculus |
| 8 | sk_Eukaryota; k_Metazoa; p_Chordata; c_Mammalia; o_Mammalia incertae |
| | sedis; f_Bovidae; g_Ovis; s_Ovis canadensis |
| 9 | sk_Eukaryota; k_Metazoa; p_Chordata; c_Actinopteri; o_Cypriniformes; f_Cyprinidae; |
| | g_Cyprinus; s_Cyprinus carpio |
| 10 | sk_Eukaryota; k_Metazoa; p_Chordata; c_Actinopteri; o_Beloniformes; f_Adrianichthyidae; |
| | g_Oryzias; s_Oryzias latipes |
| 11 | sk_Eukaryota; k_Metazoa; p_Chordata; c_Actinopteri; o_Cypriniformes; f_Cyprinidae; |
| | g_Danio; s_Danio rerio |
| 12 | sk_Bacteria; k_Bacteria incertae |
| | sedis; p_Actinobacteria; c_Actinobacteria; o_Propionibacteriales; f_Propionibacteriaceae; |
| | g_Tessaracoccus; s_Tessaracoccus sp. T2.5-30 |
| 13 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 14 | sk_Bacteria; k_Bacteria incertae |
| | sedis; p_Actinobacteria; c_Actinobacteria; o_Micrococcales; f_Intrasporangiaceae; |
| | g_Phycicoccus; s_Phycicoccus dokdonensis |
| 15 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |

TABLE 29C
Top 10 cellulolytic microorganisms under anaerobic/grass fermentation.

| | |
|---|---|
| 1 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae sedis; p_Proteobacteria; Other; Other; Other; Other; Other |
| 4 | sk_Bacteria; k_Bacteria incertae sedis; p_Candidatus Cloacimonetes; c_Candidatus |
| | Cloacimonetes incertae sedis; o_Candidatus Cloacimonetes incertae sedis; f_Candidatus |
| | Cloacimonetes incertae sedis; g_Candidatus Cloacimonas; s_Candidatus Cloacimonas |
| | acidaminovorans |
| 5 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 6 | sk_Eukaryota; k_Metazoa; p_Chordata; c_Mammalia; o_Primates; f_Hominidae; g_Homo; |
| | s_Homo sapiens |
| 7 | sk_Eukaryota; k_Metazoa; p_Chordata; c_Mammalia; o_Rodentia; f_Muridae; g_Mus; |
| | s_Mus musculus |
| 8 | sk_Eukaryota; k_Metazoa; p_Chordata; c_Mammalia; o_Mammalia incertae |
| | sedis; f_Bovidae; g_Ovis; s_Ovis canadensis |
| 9 | sk_Eukaryota; k_Metazoa; p_Chordata; c_Actinopteri; o_Cypriniformes; f_Cyprinidae; |
| | g_Cyprinus; s_Cyprinus carpio |
| 10 | sk_Eukaryota; k_Metazoa; p_Chordata; c_Actinopteri; o_Beloniformes; f_Adrianichthyidae; |
| | g_Oryzias; s_Oryzias latipes |

TABLE 30A
Top 25 cellulolytic bacterial microorganisms under aerobic/guar gum fermentation.

| | |
|---|---|
| 1 | D_0_Bacteria; D_1_Bacteroidetes; D_2 vadinHA17; Other; Other; Other; Other |
| 2 | D_0_Bacteria; D_1_Chloroflexi; D_2_Anaerolineae; D_3_Anaerolineales; |
| | D_4_Anaerolineaceae; Other; Other Most abundant in all reactors |
| 3 | D_0_Bacteria; D_1_Bacteroidetes; D_2_Sphingobacteriia; D_3_Sphingobacteriales; |
| | D_4_WCHB1-69; Other; Other |
| 4 | D_0_Bacteria; D_1_Bacteroidetes; D_2_SB-1; Other; Other; Other; Other |
| 5 | D_0_Bacteria; D_1_Chloroflexi; Other; Other; Other; Other; Other |
| 6 | D_0_Bacteria; D_1_Verrucomicrobia; D_2_OPB35 soil group; Other; Other; Other; Other |
| 7 | D_0_Bacteria; D_1_Thermotogae; D_2_Thermotogae; D_3_Thermotogales; D_4_Thermotogaceae; |
| | D_5_SC103; Other |
| 8 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 9 | D_0_Bacteria; D_1_Proteobacteria; D_2_Deltaproteobacteria; D_3_Deltaproteobacteria |
| | Incertae Sedis; D_4_Syntrophorhabdaceae; D_5_Syntrophorhabdus; Other |
| 10 | D_0_Bacteria; D_1_Firmicutes; D_2_Clostridia; D_3_Clostridiales; D_4_Family |
| | XI; D_5_Sedimentibacter; Other |
| 11 | D_0_Bacteria; D_1_Proteobacteria; D_2_Deltaproteobacteria; D_3_Desulfovibrionales; |
| | D_4_Desulfomicrobiaceae; D_5_Desulfomicrobium; Other |
| 12 | D_0_Bacteria; D_1_Firmicutes; D_2_Clostridia; D_3_Clostridiales; D_4_Lachnospiraceae; |
| | D_5_Incertae Sedis; Other |
| 13 | D_0_Bacteria; D_1_Proteobacteria; D_2_Epsilonproteobacteria; D_3_Campylobacterales; |
| | D_4_Helicobacteraceae; D_5_Sulfurovum; Other |
| 14 | D_0_Bacteria; D_1_Planctomycetes; D_2_Planctomycetacia; D_3_Planctomycetales; |
| | D_4_Planctomycetaceae; Other; Other |
| 15 | D_0_Bacteria; D_1_Chloroflexi; D_2_Anaerolineae; D_3_Anaerolineales; D_4_Anaerolineaceae; |
| | D_5_Leptolinea; Other |
| 16 | D_0_Bacteria; D_1_Spirochaetae; D_2_Spirochaetes; D_3_Spirochaetes Incertae |
| | Sedis; D_4_Unknown Family; D_5_Candidatus Cloacamonas; Other |
| 17 | D_0_Bacteria; D_1_Bacteroidetes; D_2_Bacteroidia; D_3_Bacteroidales; D_4_Porphyromonadaceae; |
| | D_5_Macellibacteroides; Other |
| 18 | D_0_Bacteria; D_1_Proteobacteria; D_2_Deltaproteobacteria; D_3_Syntrophobacterales; |
| | D_4_Syntrophaceae; Other; Other |
| 19 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 20 | D_0_Bacteria; D_1_Proteobacteria; D_2_Deltaproteobacteria; D_3_Syntrophobacterales; |
| | D_4_Syntrophaceae; D_5_Smithella; Other |
| 21 | D_0_Bacteria; D_1_Chloroflexi; D_2_Anaerolineae; D_3_Anaerolineales; D_4_Anaerolineaceae; |
| | D_5_Longilinea; Other |
| 22 | D_0_Bacteria; D_1_Candidate division BRC1; Other; Other; Other; Other; Other |
| 23 | D_0_Bacteria; D_1_Candidate division OP11; Other; Other; Other; Other; Other |
| 24 | D_0_Bacteria; D_1_Proteobacteria; D_2_Betaproteobacteria; D_3_Rhodocyclales; |
| | D_4_Rhodocyclaceae; D_5_Thauera; Other |
| 25 | D_0_Bacteria; D_1_Synergistetes; D_2_Synergistia; D_3_Synergistales; |
| | D_4_Synergistaceae; Other; Other |

TABLE 30B
Top 10 cellulolytic bacterial microorganisms under aerobic/guar gum fermentation.

| | |
|---|---|
| 1 | D_0_Bacteria; D_1_Bacteroidetes; D_2_vadinHA17; Other; Other; Other; Other |
| 2 | D_0_Bacteria; D_1_Chloroflexi; D_2_Anaerolineae; D_3_Anaerolineales; D_4_Anaerolineaceae; |
| | Other; Other Most abundant in all reactors |
| 3 | D_0_Bacteria; D_1_Bacteroidetes; D_2_Sphingobacteriia; D_3_Sphingobacteriales; |
| | D_4_WCHB1-69; Other; Other |
| 4 | D_0_Bacteria; D_1_Bacteroidetes; D_2_SB-1; Other; Other; Other; Other |
| 5 | D_0_Bacteria; D_1_Chloroflexi; Other; Other; Other; Other; Other |
| 6 | D_0_Bacteria; D_1_Verrucomicrobia; D_2_OPB35 soil group; Other; Other; Other; Other |
| 7 | D_0_Bacteria; D_1_Thermotogae; D_2_Thermotogae; D_3_Thermotogales; D_4_Thermotogaceae; |
| | D_5_SC103; Other |
| 8 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 9 | D_0_Bacteria; D_1_Proteobacteria; D_2_Deltaproteobacteria; D_3_Deltaproteobacteria |
| | Incertae Sedis; D_4_Syntrophorhabdaceae; D_5_Syntrophorhabdus; Other |
| 10 | D_0_Bacteria; D_1_Firmicutes; D_2_Clostridia; D_3_Clostridiales; D_4_Family |
| | XI; D_5_Sedimentibacter; Other |

TABLE 31A
Top 25 cellulolytic microorganisms under aerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 5 | sk_Bacteria; k_Bacteria incertae |
| | sedis; p_Proteobacteria; c_Gammaproteobacteria; |
| | o_Pseudomonadales; f_Pseudomonadaceae; g_Pseudomonas; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Primates; f_Hominidae; g_Homo; |
| | s_Homo sapiens |
| 8 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 9 | sk_Bacteria; k_Bacteria incertae |
| | sedis; p_Proteobacteria; c_Deltaproteobacteria; o_Myxococcales; f_Polyangiaceae; |
| | g_Sorangium; s_Sorangium cellulosum |
| 10 | sk_Eukaryota; k_Metazoa; p_Chordata; c_Mammalia; o_Mammalia incertae |
| | sedis; f_Bovidae; g_Ovis; s_Ovis canadensis |
| 11 | sk_Bacteria; k_Bacteria incertae |
| | sedis; p_Proteobacteria; c_Betaproteobacteria; o_Burkholderiales; Other; Other; Other |
| 12 | sk_Bacteria; k_Bacteria incertae |
| | sedis; p_Proteobacteria; c_Betaproteobacteria; o_Nitrosomonadales; f_Thiobacillaceae; |
| | g_Thiobacillus; s_Thiobacillus denitrificans |
| 13 | sk_Bacteria; k_Bacteria incertae |
| | sedis; p_Proteobacteria; c_Alphaproteobacteria; Other; Other; Other; Other |
| 14 | sk_Bacteria; k_Bacteria incertae |
| | sedis; p_Proteobacteria; c_Alphaproteobacteria; o_Rhizobiales; f_Bradyrhizobiaceae; |
| | g_Nitrobacter; s_Nitrobacter hamburgensis |
| 15 | sk_Bacteria; k_Bacteria incertae |
| | sedis; p_Proteobacteria; c_Alphaproteobacteria; o_Rhizobiales; f_Bradyrhizobiaceae; |
| | g_Rhodopseudomonas; s_Rhodopseudomonas palustris |
| 16 | sk_Eukaryota; k_Metazoa; p_Chordata; c_Actinopteri; o_Cypriniformes; f_Cyprinidae; |
| | g_Cyprinus; s_Cyprinus carpio |
| 17 | sk_Bacteria; k_Bacteria incertae |
| | sedis; p_Proteobacteria; c_Gammaproteobacteria; Other; Other; Other; Other |
| 18 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |
| 19 | sk_Bacteria; k_Bacteria incertae |
| | sedis; p_Proteobacteria; c_Betaproteobacteria; Other; Other; Other; Other |
| 20 | sk_Eukaryota; k_Metazoa; p_Chordata; c_Actinopteri; o_Beloniformes; f_Adrianichthyidae; |
| | g_Oryzias; s_Oryzias latipes |
| 21 | sk_Eukaryota; k_Metazoa; p_Chordata; c_Actinopteri; o_Cypriniformes; f_Cyprinidae; |
| | g_Danio; s_Danio rerio |
| 22 | sk_Bacteria; k_Bacteria incertae |
| | sedis; p_Proteobacteria; c_Betaproteobacteria; o_Nitrosomonadales; f_Sterolibacteriaceae; |
| | g_Sulfuritalea; s_Sulfuritalea hydrogenivorans |
| 23 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; s_Pseudomonas_citronellolis |
| 24 | sk_Eukaryota; k_Viridiplantae; p_Streptophyta; c_Liliopsida; o_Poales; f_Poaceae; g_Oryza; |
| | s_Oryza sativa |
| 25 | sk_Archaea; k_Archaea incertae |
| | sedis; p_Thaumarchaeota; c_Nitrososphaeria; o_Nitrososphaerales; f_Nitrososphaeraceae; |
| | g_Candidatus Nitrosocosmicus; s_Candidatus Nitrosocosmicus exaquare |

TABLE 31B
Top 25 cellulolytic microorganisms under aerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 5 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Primates; f_Hominidae; g_Homo; |
| | s_Homo sapiens |
| 8 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 9 | sk_Bacteria; k_Bacteria incertae |
| | sedis; p_Proteobacteria; c_Deltaproteobacteria; o_Myxococcales; f_Polyangiaceae; g_Sorangium; |
| | s_Sorangium cellulosum |
| 10 | sk_Eukaryota; k_Metazoa; p_Chordata; c_Mammalia; o_Mammalia incertae |
| | sedis; f_Bovidae; g_Ovis; s_Ovis canadensis |
| 11 | sk_Bacteria; k_Bacteria incertae |
| | sedis; p_Proteobacteria; c_Betaproteobacteria; o_Burkholderiales; Other; Other; Other |
| 12 | sk_Bacteria; k_Bacteria incertae |
| | sedis; p_Proteobacteria; c_Betaproteobacteria; o_Nitrosomonadales; f_Thiobacillaceae; |
| | g_Thiobacillus; s_Thiobacillus denitrificans |
| 13 | sk_Bacteria; k_Bacteria incertae |
| | sedis; p_Proteobacteria; c_Alphaproteobacteria; Other; Other; Other; Other |
| 14 | sk_Bacteria; k_Bacteria incertae |
| | sedis; p_Proteobacteria; c_Alphaproteobacteria; o_Rhizobiales; f_Bradyrhizobiaceae; |
| | g_Nitrobacter; s_Nitrobacter hamburgensis |
| 15 | sk_Bacteria; k_Bacteria incertae |
| | sedis; p_Proteobacteria; c_Alphaproteobacteria; o_Rhizobiales; f_Bradyrhizobiaceae; |
| | g_Rhodopseudomonas; s_Rhodopseudomonas palustris |

TABLE 31C
Top 10 cellulolytic microorganisms under aerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 5 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 6 | sk_Eukaryota; k_Metazoa; p_Chordata; c_Mammalia; o_Rodentia; f_Muridae; g_Mus; |
| | s_Mus musculus |
| 7 | sk_Eukaryota; k_Metazoa; p_Chordata; c_Mammalia; o_Primates; f_Hominidae; g_Homo; |
| | s_Homo sapiens |
| 8 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 9 | sk_Bacteria; k_Bacteria incertae |
| | sedis; p_Proteobacteria; c_Deltaproteobacteria; o_Myxococcales; f_Polyangiaceae; |
| | g_Sorangium; s_Sorangium cellulosum |
| 10 | sk_Eukaryota; k_Metazoa; p_Chordata; c_Mammalia; o_Mammalia incertae |
| | sedis; f_Bovidae; g_Ovis; s_Ovis canadensis |

TABLE 32A
Top 25 cellulolytic bacterial microorganisms under anaerobic/guar gum fermentation.

| | |
|---|---|
| 1 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 2 | D_0_Bacteria; D_1_Bacteroidetes; D_2_vadinHA17; Other; Other; Other; Other |
| 3 | D_0_Bacteria; D_1_Chloroflexi; D_2_Anaerolineae; D_3_Anaerolineales; D_4_Anaerolineaceae; |
| | Other; Other Most abundant in all reactors |
| 4 | D_0_Bacteria; D_1_Proteobacteria; D_2_Epsilonproteobacteria; D_3_Campylobacterales; |
| | D_4_Helicobacteraceae; D_5_Sulfurovum; Other |
| 5 | D_0_Bacteria; D_1_Bacteroidetes; D_2_Sphingobacteriia; D_3_Sphingobacteriales; |
| | D_4_WCHB1-69; Other; Other |
| 6 | D_0_Bacteria; D_1_Bacteroidetes; D_2_SB-1; Other; Other; Other; Other |
| 7 | D_0_Bacteria; D_1_Chloroflexi; Other; Other; Other; Other; Other |
| 8 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 9 | D_0_Bacteria; D_1_Verrucomicrobia; D_2_OPB35 soil group; Other; Other; Other; Other |
| 10 | D_0_Bacteria; D_1_Thermotogae; D_2_Thermotogae; D_3_Thermotogales; D_4_Thermotogaceae; |
| | D_5_SC103; Other |
| 11 | D_0_Bacteria; D_1_Proteobacteria; D_2_Deltaproteobacteria; D_3_Deltaproteobacteria |
| | Incertae Sedis; D_4_Syntrophorhabdaceae; D_5_Syntrophorhabdus; Other |
| 12 | D_0_Bacteria; D_1_Proteobacteria; D_2_Deltaproteobacteria; D_3_Desulfovibrionales; |
| | D_4_Desulfomicrobiaceae; D_5_Desulfomicrobium; Other |
| 13 | D_0_Bacteria; D_1_Firmicutes; D_2_Clostridia; D_3_Clostridiales; D_4_Lachnospiraceae; |
| | D_5_Incertae Sedis; Other |
| 14 | D_0_Bacteria; D_1_Firmicutes; D_2_Clostridia; D_3_Clostridiales; D_4_Family |
| | XI; D_5_Sedimentibacter; Other |
| 15 | D_0_Bacteria; D_1_Chloroflexi; D_2_Anaerolineae; D_3_Anaerolineales; D_4_Anaerolineaceae; |
| | D_5_Leptolinea; Other |
| 16 | D_0_Bacteria; D_1_Bacteroidetes; D_2_Bacteroidia; D_3_Bacteroidales; D_4_Porphyromonadaceae; |
| | D_5_Paludibacter; Other |
| 17 | D_0_Bacteria; D_1_Planctomycetes; D_2_Planctomycetacia; D_3_Planctomycetales; |
| | D_4_Planctomycetaceae; Other; Other |
| 18 | D_0_Bacteria; D_1_Synergistetes; D_2_Synergistia; D_3_Synergistales; D_4_Synergistaceae; |
| | D_5_Thermovirga; Other |
| 19 | D_0_Bacteria; D_1_Acidobacteria; D_2_Holophagae; D_3_Subgroup 10; D_4_ABS-19; |
| | Other; Other |
| 20 | D_0_Bacteria; D_1_Proteobacteria; D_2_Deltaproteobacteria; D_3_Syntrophobacterales; |
| | D_4_Syntrophaceae; Other; Other |
| 21 | D_0_Bacteria; D_1_Candidate division BRC1; Other; Other; Other; Other; Other |
| 22 | D_0_Bacteria; D_1_Proteobacteria; D_2_Deltaproteobacteria; D_3_Syntrophobacterales; |
| | D_4_Syntrophaceae; D_5_Smithella; Other |
| 23 | D_0_Bacteria; D_1_Chloroflexi; D_2_Anaerolineae; D_3_Anaerolineales; D_4_Anaerolineaceae; |
| | D_5_Longilinea; Other |
| 24 | D_0_Bacteria; D_1_Synergistetes; D_2_Synergistia; D_3_Synergistales; D_4_Synergistaceae; |
| | Other; Other |
| 25 | D_0_Bacteria; D_1_Spirochaetae; D_2_Spirochaetes; D_3_Spirochaetes Incertae |
| | Sedis; D_4_Unknown Family; D_5_Candidatus Cloacamonas; Other |

TABLE 32B
Top 10 cellulolytic bacterial microorganisms under anaerobic/guar gum fermentation.

| | |
|---|---|
| 1 | D_0_Bacteria; D_1_Bacteroidetes; D_2_Bacteroidia; D_3_Bacteroidales; D_4_Bacteroidaceae; |
| | D_5_Bacteroides; Other 4C_PE Reactor |
| 2 | D_0_Bacteria; D_1_Bacteroidetes; D_2_vadinHA17; Other; Other; Other; Other |
| 3 | D_0_Bacteria; D_1_Chloroflexi; D_2_Anaerolineae; D_3_Anaerolineales; D_4_Anaerolineaceae; |
| | Other; Other Most abundant in all reactors |
| 4 | D_0_Bacteria; D_1_Proteobacteria; D_2_Epsilonproteobacteria; D_3_Campylobacterales; |
| | D_4_Helicobacteraceae; D_5_Sulfurovum; Other |
| 5 | D_0_Bacteria; D_1_Bacteroidetes; D_2_Sphingobacteriia; D_3_Sphingobacteriales; |
| | D_4_WCHB1-69; Other; Other |
| 6 | D_0_Bacteria; D_1_Bacteroidetes; D_2_SB-1; Other; Other; Other; Other |
| 7 | D_0_Bacteria; D_1_Chloroflexi; Other; Other; Other; Other; Other |
| 8 | D_0_Bacteria; D_1_Bacteroidetes; D_2_Bacteroidia; D_3_Bacteroidales; D_4_Rikenellaceae; |
| | D_5_vadinBC27 wastewater-sludge group; Other |
| 9 | D_0_Bacteria; D_1_Verrucomicrobia; D_2_OPB35 soil group; Other; Other; Other; Other |
| 10 | D_0_Bacteria; D_1_Thermotogae; D_2_Thermotogae; D_3_Thermotogales; D_4_Thermotogaceae; |
| | D_5_SC103; Other |

TABLE 33A
Top 25 cellulolytic microorganisms under an anaerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 5 | sk_Bacteria; k_Bacteria incertae |
| | sedis; p_Actinobacteria; c_Actinobacteria; o_Streptomycetales; f_Streptomycetaceae; g_Streptomyces; Other |
| 6 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 7 | sk_Bacteria; k_Bacteria incertae |
| | sedis; p_Proteobacteria; c_Betaproteobacteria; o_Burkholderiales; Other; Other; Other |
| 8 | sk_Bacteria; k_Bacteria incertae |
| | sedis; p_Proteobacteria; c_Betaproteobacteria; o_Nitrosomonadales; f_Sterolibacteriaceae; |
| | g_Sulfuritalea; s_Sulfuritalea hydrogenivorans |
| 9 | sk_Bacteria; k_Bacteria incertae |
| | sedis; p_Proteobacteria; c_Betaproteobacteria; o_Nitrosomonadales; f_Thiobacillaceae; |
| | g_Thiobacillus; s_Thiobacillus denitrificans |
| 10 | sk_Bacteria; k_Bacteria incertae |
| | sedis, p_Proteobacteria; c_Betaproteobacteria; Other; Other; Other; Other |
| 11 | sk_Eukaryota; k_Metazoa; p_Chordata; c_Mammalia; o_Primates; f_Hominidae; g_Homo; |
| | s_Homo sapiens |
| 12 | sk_Bacteria; k_Bacteria incertae |
| | sedis; p_Proteobacteria; c_Deltaproteobacteria; o_Myxococcales; f_Polyangiaceae; |
| | g_Sorangium; s_Sorangium cellulosum |
| 13 | sk_Eukaryota; k_Metazoa; p_Chordata; c_Mammalia; o_Rodentia; f_Muridae; g_Mus; |
| | s_Mus musculus |
| 14 | sk_Eukaryota; k_Metazoa; p_Chordata; c_Mammalia; o_Mammalia incertae |
| | sedis; f_Bovidae; g_Ovis; s_Ovis canadensis |
| 15 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |
| 16 | sk_Bacteria; k_Bacteria incertae |
| | sedis; p_Proteobacteria; c_Gammaproteobacteria; Other; Other; Other; Other |
| 17 | sk_Bacteria; k_Bacteria incertae |
| | sedis; p_Firmicutes; c_Clostridia; o_Clostridiales; f_Clostridiaceae; g_Clostridium; |
| | s_Clostridium butyricum |
| 18 | sk_Bacteria; k_Bacteria incertae |
| | sedis; p_Proteobacteria; c_Betaproteobacteria; o_Rhodocyclales; f_Rhodocyclaceae; |
| | g_Azospira; s_Azospira oryzae |
| 19 | sk_Bacteria; k_Bacteria incertae |
| | sedis; p_Proteobacteria; c_Alphaproteobacteria; Other; Other; Other; Other |
| 20 | sk_Bacteria; k_Bacteria incertae |
| | sedis; p_Proteobacteria; c_Betaproteobacteria; o_Rhodocyclales; f_Zoogloeaceae; |
| | g_Thauera; s_Thauera sp. K11 |
| 21 | sk_Bacteria; k_Bacteria incertae |
| | sedis; p_Proteobacteria; c_Deltaproteobacteria; o_Myxococcales; f_Labilitrichaceae; |
| | g_Labilithrix; s_Labilithrix luteola |
| 22 | sk_Bacteria; k_Bacteria incertae |
| | sedis; p_Proteobacteria; c_Betaproteobacteria; o_Burkholderiales; f_Burkholderiaceae; |
| | Other; Other |
| 23 | sk_Bacteria; k_Bacteria incertae |
| | sedis; p_Proteobacteria; c_Alphaproteobacteria; o_Rhizobiales; f_Bradyrhizobiaceae; |
| | g_Rhodopseudomonas; s_Rhodopseudomonas palustris |
| 24 | sk_Bacteria; k_Bacteria incertae |
| | sedis; p_Proteobacteria; c_Betaproteobacteria; o_Rhodocyclales; f_Zoogloeaceae; |
| | g_Thauera; s_Thauera sp. MZ1T |
| 25 | sk_Eukaryota; k_Viridiplantae; p_Streptophyta; c_Liliopsida; o_Poales; f_Poaceae; g_Oryza; |
| | s_Oryza sativa |

TABLE 33B
Top 15 cellulolytic microorganisms under an anaerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 5 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 6 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 7 | sk_Bacteria; k_Bacteria incertae |
| | sedis; p_Proteobacteria; c_Betaproteobacteria; o_Burkholderiales; Other; Other; Other |
| 8 | sk_Bacteria; k_Bacteria incertae |
| | sedis; p_Proteobacteria; c_Betaproteobacteria; o_Nitrosomonadales; f_Sterolibacteriaceae; |
| | g_Sulfuritalea; s_Sulfuritalea hydrogenivorans |
| 9 | sk_Bacteria; k_Bacteria incertae |
| | sedis; p_Proteobacteria; c_Betaproteobacteria; o_Nitrosomonadales; f_Thiobacillaceae; |
| | g_Thiobacillus; s_Thiobacillus denitrificans |
| 10 | sk_Bacteria; k_Bacteria incertae |
| | sedis; p_Proteobacteria; c_Betaproteobacteria; Other; Other; Other; Other |
| 11 | sk_Eukaryota; k_Metazoa; p_Chordata; c_Mammalia; o_Primates; f_Hominidae; g_Homo; |
| | s_Homo sapiens |
| 12 | sk_Bacteria; k_Bacteria incertae |
| | sedis; p_Proteobacteria; c_Deltaproteobacteria; o_Myxococcales; f_Polyangiaceae; |
| | g_Sorangium; s_Sorangium cellulosum |
| 13 | sk_Eukaryota; k_Metazoa; p_Chordata; c_Mammalia; o_Rodentia; f_Muridae; g_Mus; |
| | s_Mus musculus |
| 14 | sk_Eukaryota; k_Metazoa; p_Chordata; c_Mammalia; o_Mammalia incertae |
| | sedis; f_Bovidae; g_Ovis; s_Ovis canadensis |
| 15 | sk_Bacteria; k_Bacteria incertae |
| | sedis; p_Actinobacteria; c_Actinobacteria; o_Corynebacteriales; f_Mycobacteriaceae; |
| | g_Mycobacterium; Other |

TABLE 33C
Top 10 cellulolytic microorganisms under an anaerobic/guar gum fermentation.

| | |
|---|---|
| 1 | sk_Bacteria; k_Bacteria incertae sedis; p_Proteobacteria; Other; Other; Other; Other; Other |
| 2 | sk_Bacteria; k_Bacteria incertae |
| | sedis; p_Actinobacteria; c_Actinobacteria; Other; Other; Other; Other |
| 3 | sk_Bacteria; k_Bacteria incertae |
| | sedis; p_Proteobacteria; c_Betaproteobacteria; o_Burkholderiales; f_Burkholderiaceae; |
| | g_Burkholderia; Other |
| 4 | sk_Archaea; k_Archaea incertae |
| | sedis; p_Euryarchaeota; c_Methanomicrobia; o_Methanosarcinales; f_Methanosaetaceae; |
| | g_Methanothrix; s_Methanothrix soehngenii |
| 5 | sk_Bacteria; k_Bacteria incertae |
| | sedis; p_Actinobacteria; c_Actinobacteria; o_Streptomycetales; f_Streptomycetaceae; |
| | g_Streptomyces; Other |
| 6 | sk_Bacteria; k_Bacteria incertae |
| | sedis; p_Proteobacteria; c_Gammaproteobacteria; o_Pseudomonadales; f_Pseudomonadaceae; |
| | g_Pseudomonas; Other |
| 7 | sk_Bacteria; k_Bacteria incertae |
| | sedis; p_Proteobacteria; c_Betaproteobacteria; o_Burkholderiales; Other; Other; Other |
| 8 | sk_Bacteria; k_Bacteria incertae |
| | sedis; p_Proteobacteria; c_Betaproteobacteria; o_Nitrosomonadales; f_Sterolibacteriaceae; |
| | g_Sulfuritalea; s_Sulfuritalea hydrogenivorans |
| 9 | sk_Bacteria; k_Bacteria incertae |
| | sedis; p_Proteobacteria; c_Betaproteobacteria; o_Nitrosomonadales; f_Thiobacillaceae; |
| | g_Thiobacillus; s_Thiobacillus denitrificans |
| 10 | sk_Bacteria; k_Bacteria incertae |
| | sedis; p_Proteobacteria; c_Betaproteobacteria; Other; Other; Other; Other |

TABLE 34

| | Aerobic | Anaerobic | |
|---|---|---|---|
| Feedstock, | Average of all tests; VFA Yield, mg/g with | | Avg |
| g | 5.18% Moisture Content | | hrs |
| Grass¹ | | | |
| 1 | 36.7 | 70.9 | |
| 2 | 43.7 | 103.9 | 92.7 |
| 4 | 101.2 | 122.6 | 104.2 |
| 6 | 69.4 | 121.1 | 106.4 |
| Guar gum² | | | |
| 1 | 231.2 | 332.3 | |
| 2 | 148.5 | 301.6 | |
| 4 | 94.8 | 228 | |
| 0.5 | 60 | 471.4 | |

¹Yields are based on 5.18% moisture in grass or 94.82% dry basis.
²Yields are based on 5.21% moisture in guar gum or 94.79% dry basis.

TABLE 35

| | |
|---|---|
| Aerobic | Anaerobic |

| | | |
|---|---|---|
| Feedstock, | Average of all tests; VFA | Avg |
| g | Yield, mg/g dry basis | hrs |

| Grass¹ | | | | |
|---|---|---|---|---|
| | 1 | 38.7 | 74.8 | |
| | 2 | 46 | 109.6 | 92.7 |
| | 4 | 106.8 | 129.3 | 104.2 |
| | 6 | 73.2 | 127.7 | 106.4 |
| Guar gum¹ | | | | |
| | 1 | 243.9 | 350.6 | |
| | 2 | 156.7 | 318.2 | |
| | 4 | 100 | 240.5 | |
| | 0.5 | 63.3 | 497.3 | |

¹Yields were based on 5.18% moisture on 100.00% dry basis.

### Rama Industry Analysis for Guar Gum and EMRD for Grass.

The guar gum powder used in the experiment was a powder product, 200 mesh 35-40 cPs (centi-poise; a unit for viscosity). The analysis results presented below was conducted and provided by Rama Industries of India.

TABLE 36

| Parameter with unit if applicable | Value |
|---|---|
| Physical appearance | Light cream-colored |
| | homogenous powder |
| Granulometry; % passing through 100 mesh | Nil |
| Granulometry; % passing through 200 mesh | 99.20 |
| Viscosity, cPs | |
| After 3 minutes | 35.5 |
| After 3 minutes | 40.5 |
| After 3 minutes | 41.0 |
| Moisture, % | 5.21 |
| Ash, % | 0.71 |
| Protein, % | 3.81 |
| Air, % | 2.49 |
| Glactomannan, % | 87.78 |
| pH | 7.02 |

TABLE 37
Analysis conducted by MWRDGC on Grass moisture.

| Parameter, unit | |
|---|---|
| Total Solids, mg/L | 830 |
| Total Volatile Solids, mg/L | 707 |
| Chemical Oxygen demand, mg/L | 772 |

The first four, under the “Blended Grass” grouping, were taken from the blended stock of grass that are sitting in a 5-gallon pail on the benchtop. This stock had previously been dried in the hood prior to blending. The blended grass was then placed into the drying oven (104 degrees C. for 24 hours) and the resulting percent moisture between the hood dried grass and the oven dried grass is 5.2%.

The next four samples used fresh cut grass. The first two were placed into the drying oven (104 degrees C. for 24 hours) and the resulting percent moisture of 68.1%. The last two were first placed into the hood to dry (66.9% moisture), then into the oven drying oven (104 degrees C. for 24 hours) with a final percent moisture of 68.3% compared to the original fresh cut grass. The difference between hood dried and oven dried is only around 1.4% (68.3-66.9), versus the 5.2% in the first experiment using the blended grass. However, the difference is likely due to the blended grass holding some moisture from the air while sitting in the 5-gallon pail.

TABLE 38
Percent total solids determination on grass samples.

| | Pan | Grass + | Grass | Grass + | Grass | % | Grass + | Grass | % | |
|---|---|---|---|---|---|---|---|---|---|---|
| | Weight | Pan | Only | Pan | Only | Moisture | Pan | Only | Moisture | Average |
| | (g) | (g, Initial) | (g, Initial) | (g, Hood) | (g, Hood) | (Hood) | (g, Oven) | (g, Oven) | (Oven) | % |
| Blended | 5.7849 | NA | NA | 7.8037 | 2.0188 | NA | 7.6984 | 1.9135 | 5.22 | 5.18 |
| Grass | 5.7395 | NA | NA | 7.7412 | 2.0017 | NA | 7.6390 | 1.8995 | 5.11 | |
| | 5.8329 | NA | NA | 7.8414 | 2.0085 | NA | 7.7378 | 1.9049 | 5.16 | |
| | 5.5424 | NA | NA | 7.5559 | 2.0135 | NA | 7.4502 | 1.9078 | 5.25 | |
| Fresh | 5.5416 | 8.4932 | 2.9516 | NA | NA | NA | 6.4552 | 0.9136 | 69.05 | 68.21 |
| Grass | 5.9936 | 9.0277 | 3.0341 | NA | NA | NA | 6.9900 | 0.9964 | 67.16 | |
| | 5.6423 | 10.5426 | 4.9003 | 7.2233 | 1.5810 | 67.74 | 7.1557 | 1.5134 | 69.12 | |
| | 5.6368 | 10.6124 | 4.9756 | 7.3270 | 1.6902 | 66.03 | 7.2538 | 1.6170 | 67.50 | |

0.5 gal tap wtr+dry cut grass in 1 gal jar on 5/20 at 9:30 am; sampled 5/31 and 7/7,

Guar Gum 2.4 g+500 mL Distilled Water

TABLE 39
Preliminary experiments with important characteristics.

| | | 11 days of | 48 days of | 47 days of |
|---|---|---|---|---|
| | | soaking in 0.5 | soaking in 0.5 | soaking in 0.5 |
| | | gal tap water in | gal tap water in | gal tap water in |
| | | one gallon jar | one gallon jar | one gallon jar |
| Date of analysis | | May 31, 2016* | Jul. 8, 2016*** | Jul. 7, 2016** |
| Dosage tested | | fistful/0.5 gal or 1.90 L | | 2.4 g/500 mL |
| Parameter | Unit | Grass | Grass | Guar Gum |
| Temperature | C. | 11 | 22 | 24.5 |
| pH, | pH unit | 6.1 | 6.5 | 5.3 |
| TSW % | % | 0.27 | 0.31 | 0.59 |
| By calculation VS | % | 63.33 | 72.45 | 100.00 |
| ASH % | % | 36.67 | 27.55 | 0.00 |
| COD | mg/L | 3861 | 3232 | 6516 |
| Sol-COD | mg/L | 2771 | 2837 | 3368 |
| % Soluble COD | % | 72 | 88 | 52 |
| Tot Phos | mg/L | 39.39 | — | — |
| VOL ACIDS | mg/L | 2068 | — | — |
| AV_XBOD | mg/L | 2551 | 1651 | 1580 |
| Cd | mg/Kg | | — | — |
| Cr | mg/Kg | | — | — |
| Cu | mg/Kg | | — | — |
| Fe | mg/Kg | | — | — |
| Ni | mg/Kg | | — | — |
| Pb | mg/Kg | | — | — |
| Zn | mg/Kg | | — | — |
| Final Hg | ug/Kg | | — | — |
| Final Hg | ug/L | | <0.20 | <0.20 |
| Cd | mg/L | | <0.0050 | |
| Cr | mg/L | | <0.0050 | |
| Fe | mg/L | | 2.92 | |
| Ni | mg/L | | <0.0100 | |
| Pb | mg/L | | <0.050 | |
| Zn | mg/L | | 0.055 | |
| Cu | mg/L | | 0.0401 | |

The breadth and scope of the present disclosure should not be limited by any of the above-described exemplary embodiments, but should be defined only in accordance with the following claims and their equivalents.

1. A method of producing a volatile fatty acid, the method comprising fermenting a cellulosic feedstock present in a fermentation solution, the fermentation solution comprising:
cellulolytic microorganisms; and
the cellulosic feedstock,
wherein the cellulosic feedstock comprises grass, leaves, phragmites, cattails, guar gum, or a combination thereof, and wherein the fermentation of the cellulosic feedstock generates volatile fatty acids,
optionally, wherein the cellulosic feedstock comprises grass or guar gum.

2. The method of claim 1, wherein the cellulolytic microorganisms are supplied to the fermentation solution in an inoculum comprising the cellulolytic microorganisms;
optionally, wherein the composition of identities of and/or the metabolic pathways utilized by the cellulolytic microorganisms supplied to the fermentation solution have been previous adapted to the type of cellulosic feedstock and/or fermentation conditions to be used.

3-10. (canceled)

11. The method of claim 1, wherein the amount of the cellulosic feedstock is:
from about 10 g/L to 60 g/L in the fermentation solution, from about 20 g/L to 60 g/L in the fermentation solution, from about 20 g/L to 50 g/L in the fermentation solution, from about 30 g/L to 50 g/L in the fermentation solution, from about 35 g/L to 45 g/L in the fermentation solution, or about 40 g/L in the fermentation solution, wherein the feedstock is grass fermented under aerobic or anaerobic conditions;
from about 5 g/L and 40 g/L in the fermentation solution, from about 5 g/L and 25 g/L in the fermentation solution, from about 5 g/L and 15 g/L in the fermentation solution, or about 10 g/L, wherein the feedstock is guar gum fermented under aerobic conditions; or
from about 1 g/L and 40 g/L in the fermentation solution, from about 1 g/L and 25 g/L in the fermentation solution, from about 1 g/L and 10 g/L in the fermentation solution, from about 3 g/L and 7 g/L in the fermentation solution, from about 4 g/L and 6 g/L in the fermentation solution, or about 5 g/L in the fermentation solution, wherein the feed stock is guar gum fermented under anaerobic conditions.

12. (canceled)

13. The method of claim 1, wherein the cellulosic feedstock is fermented at a pH of from about pH 5.5 to pH 6.5, from about pH 6.0 to pH 6.4, or from about pH 6.1 to pH 6.3;
optionally at a pH of about pH 6.2;
optionally, wherein the pH of the fermentation inhibits the growth of methanogens and/or inhibits biogas production.

14. (canceled)

15. The method of claim 1, wherein the cellulosic feedstock is fermented at a pH of from about pH 6.3 to pH 7.5, from about pH 6.3 to pH 7.2, from about pH 6.4 to pH 7.2, from about pH 6A to pH 7.5, from about pH 6.5 to pH 7.2, or from about pH 6.5 to pH 7.5.

16-17. (canceled)

18. The method of claim 1, wherein the fermentation of the cellulosic feedstock during anaerobic fermentation further generates a biogas;
optionally wherein the biogas is methane and/or carbon dioxide.

19-30. (canceled)

31. The method of claim 1, wherein the cellulosic feedstock comprises grass and the fermentation is anaerobic;
optionally wherein the grass is not switchgrass.

32. The method of claim 31, wherein at least 1, 2, 3, 4, 5, 10, 15, 20, or more of the top 25 most abundant types of cellulolytic bacterial microorganisms in the fermentation solution and/or in the inoculum are selected from the group consisting of the cellulolytic bacterial microorganisms in Table 28A;
optionally, wherein the top 25 most abundant types of cellulolytic bacterial microorganisms in the fermentation solution and/or in the inoculum consist of the cellulolytic bacterial microorganisms in Table 28A;
optionally, wherein the cellulolytic bacterial microorganisms in the fermentation solution and/or in the inoculum have been adapted to fermentation of grass under anaerobic conditions.

33. The method of claim 31, wherein at least 1, 2, 3, 4, 5, 6, 7, 8, 9 or all 10 of the top 10 most abundant types of cellulolytic bacterial microorganisms in the fermentation solution and/or in the inoculum are selected from the group consisting of the cellulolytic bacterial microorganisms in Table 28B;
optionally, wherein the cellulolytic bacterial microorganisms in the fermentation solution and/or in the inoculum have been adapted to fermentation of grass under anaerobic conditions.

34. The method of claim 31, wherein at least 1, 2, 3, 4, or all 5 of the top 5 most abundant types of cellulolytic bacterial microorganisms in the fermentation solution and/or in the inoculum are selected from the group consisting of:
D_0_Bacteria; D_1_Chloroflexi; D_2_Anaerolineae; D_3_Anaerolineales; D_4 Anaerolineaceae; Other; Other,
D_0_Bacteria; D_1_Bacteroidetes; D_2_SB-1; Other; Other; Other; Other,
D_0_Bacteria; D_1_Bacteroidetes; D_2_Bacteroidia; D_3_Bacteroidales; D_4 Rikenellaceae; D_5_vadinBC27 wastewater-sludge group; Other,
D_0_Bacteria; D_1_Bacteroidetes; D_2_vadinHA17; Other; Other; Other; Other, and
D_0_Bacteria; D_1 Synergistetes; D_2 Synergistia; D_3 Synergistales; D_4 Synergistaceae; D_5 Thermovirga; Other;
optionally, wherein the cellulolytic bacterial microorganisms in the fermentation solution and/or in the inoculum have been adapted to fermentation of grass under anaerobic conditions.

35. The method of claim 31, wherein at least 1, 2, or all 3 of the top 3 most abundant types of cellulolytic bacterial microorganisms in the fermentation solution and/or in the inoculum are selected from the group consisting of:
D_0_Bacteria; D_1_Chloroflexi; D_2_Anaerolineae; D_3 Anaerolineales; D_4 Anaerolineaceae; Other; Other,
D_0_Bacteria; D_1_Bacteroidetes; D_2_SB-1; Other; Other; Other; Other, and
D_0_Bacteria; D_1_Bacteroidetes; D_2_Bacteroidia; D_3_Bacteroidales; D_4 Rikenellaceae; D_5_vadinBC27 wastewater-sludge group; Other;
optionally, wherein the cellulolytic bacterial microorganisms in the fermentation solution and/or in the inoculum have been adapted to fermentation of grass under anaerobic conditions.

36. The method of claim 31, wherein at least 1 or both of the top 2 most abundant types of cellulolytic bacterial microorganisms in the fermentation solution and/or in the inoculum are selected from the group consisting of:
D_0_Bacteria; D_1_Chloroflexi; D_2_Anaerolineae; D_3 Anaerolineales; D_4 Anaerolineaceae; Other; Other, and
D_0_Bacteria; D_1_Bacteroidetes; D_2_SB-1; Other; Other; Other; Other;
optionally, wherein the cellulolytic bacterial microorganisms in the fermentation solution and/or in the inoculum have been adapted to fermentation of grass under anaerobic conditions.

37. The method of claim 31, wherein at least 1, 2, 3, 4, 5, 10, 15, 20, or more of the top 25 most abundant types of cellulolytic microorganisms in the fermentation solution and/or in the inoculum are selected from the group consisting of the cellulolytic microorganisms in Table 29A;
optionally, wherein the top 25 most abundant types of cellulolytic microorganisms in the fermentation solution and/or in the inoculum consist of the cellulolytic microorganisms in Table 29A;
optionally, wherein the cellulolytic microorganisms in the fermentation solution and/or in the inoculum have been adapted to fermentation of grass under anaerobic conditions.

38. The method of claim 31, wherein at least 1, 2, 3, 4, 5, 6, 7, 8, 9, 10, 11, 12, 13, 14 or all 15 of the top 15 most abundant types of cellulolytic microorganisms in the fermentation solution and/or in the inoculum are selected from the group consisting of the cellulolytic microorganisms in Table 29B;
optionally, wherein the cellulolytic microorganisms in the fermentation solution and/or in the inoculum have been adapted to fermentation of grass under anaerobic conditions.

39. The method of claim 31, wherein at least 1, 2, 3, 4, 5, 6, 7, 8, 9 or all 10 of the top 10 most abundant types of cellulolytic microorganisms in the fermentation solution and/or in the inoculum are selected from the group consisting of the cellulolytic microorganisms in Table 29C;
optionally, wherein the cellulolytic microorganisms in the fermentation solution and/or in the inoculum have been adapted to fermentation of grass under anaerobic conditions.

40. The method of claim 31, wherein at least 1, 2, 3, 4, or all 5 of the top 5 most abundant types of cellulolytic microorganisms in the fermentation solution and/or in the inoculum are selected from the group consisting of:
sk_Archaea; k_Archaea incertaesedis; p_Euryarchaeota; c_Methanomicrobia; o_Methanosarcinales; f_Methanosaetaceae; g_Methanothrix; s_Methanothrix soehngenii,
sk_Bacteria; k_Bacteria incertaesedis; p_Actinobacteria; c_Actinobacteria; Other; Other; Other; Other,
sk_Bacteria; k_Bacteria incertaesedis; p_Proteobacteria; Other; Other; Other; Other; Other,
sk_Bacteria; k_Bacteria incertae sedis; p_Candidatus Cloacimonetes; c_Candidatus Cloacimonetes incertae sedis; o_Candidatus Cloacimonetes incertae sedis; f_Candidatus Cloacimonetes incertae sedis; g_Candidatus Cloacimonas; s_Candidatus Cloacimonas acidaminovorans, and
sk_Bacteria; k_Bacteria incertaesedis; p_Actinobacteria; c_Actinobacteria; o_Streptomycetales; f_Streptomycetaceae; g_Streptomyces; Other;
optionally, wherein the cellulolytic microorganisms in the fermentation solution and/or in the inoculum have been adapted to fermentation of grass under anaerobic conditions.

41-60. (canceled)

61. The method of claim 2, wherein the cellulolytic microorganisms in the inoculum are derived from a seed source reservoir.

62. The method of claim 61, wherein the seed source reservoir is an anaerobic mesophilic digester draw from a municipal sewage treatment operation.

63. The method of claim 1 comprising deriving cellulolytic microorganisms from a seed source reservoir for use in the fermentation of the cellulosic feedstock;
optionally, wherein derivation comprises fermentation of the cellulolytic microorganisms from a seed source reservoir for at least 1 day, 2 days, 3 days, 4 days, 5 days, 6 day, 7 days, at least 8 days, at least 10 days, at least 14 days, at least 21 days, at least 28 days, or at least 30 days.

64. The method of claim 63, wherein during derivation, the composition of the identities of and/or the metabolic pathways utilized by the cellulolytic microorganisms is adapted to the type of cellulosic feedstock and/or fermentation conditions to be used.

65-77. (canceled)


SEQUENCE LISTING (2 sequences)

SEQ ID NO: 1 - DNA, 19 bp, Artificial Sequence
  Synthetic
gtgccagcmgccgcggtaa

SEQ ID NO: 2 - DNA, 20 bp, Artificial Sequence
  Synthetic
ccgycaattymtttragttt